(19) United States
(12) Patent Application Publication
(10) Pub. No.: US 2022/0370495 A1
(43) Pub. Date: Nov. 24, 2022

(54) IMMUNE CELLS FOR ADOPTIVE CELL THERAPIES

(71) Applicant: Board of Regents, The University of Texas System, Austin, TX (US)

(72) Inventors: Sattva S NEELAPU, Houston, TX (US); Jingwei LIU, Houston, TX (US)

(73) Assignee: Board of Regents, The University of Texas System, Austin, TX (US)

(21) Appl. No.: 17/635,560

(22) PCT Filed: Aug. 19, 2020
(86) PCT No.: PCT/US2020/047078

§ 371 Date: Feb. 15, 2022

Related U.S. Application Data
(60) Provisional application No. 62/889,662, filed on Aug. 21, 2019.

(51) Int. Cl. A61K 35/17; A61K 38/20; A61P 35/00; C07K 14/725; C12N 5/078

(52) U.S. Cl. CPC A61K 35/17; A61K 38/2013; A61K 38/2086; A61P 35/00; C07K 14/7051; C12N 5/0634; C12N 2770/20034; C07K 2319/03; C12N 2510/04

(57) ABSTRACT

Provided are methods for the production of infinite immune cells with an increased lifespan and high proliferation rates by engineering them to express BCL6 and a cell survival-promoting gene. Further provided herein are methods for the production and use of the infinite immune cells for the treatment of diseases, such as cancer.

This application claims priority to U.S. Provisional Patent Application Ser. No. 62/889,662, filed on Aug. 21, 2019, which is incorporated by reference herein in its entirety.

### BACKGROUND

### 1. Field

The present disclosure relates generally at least to the fields of molecular biology, cell biology, immunology, and medicine. More particularly, it concerns methods of producing infinite immune cells and methods of use thereof.

### 2. Description of Related Art

NK and T cells are two types of commonly used cytotoxic lymphocytes in adoptive cell therapy studies. NK and T cell derived CAR-NK cells, CAR T cells, TCR-transduced T cells, and T cells with endogenous T-cell receptors specific for microbial or tumor antigens are highly promising approaches for the treatment of both hematological malignancies and solid tumors. Three CAR-T cell products targeting CD19 have recently been approved by the FDA for B cell malignancies, and more products are in development. The generation of both TCR-T cell and CAR-T cell therapy products currently is a multi-step process that requires isolation of T cells from healthy donors or patients first, followed by introduction of TCRs or CARs in those T cells using viral or non-viral vectors, and expansion of the genetically modified T cells in vitro prior to infusion into the patients. The generation of microbial and tumor antigen-specific T cells similarly is a multi-step process that requires collection of T cells from healthy donors or patients first, followed by isolation and/or stimulation in vitro with microbial or tumor antigenic peptides or proteins, and expansion of the T cells in vitro prior to infusion into the patients.

This makes it expensive, cumbersome, and time-consuming to make the product for each patient. Furthermore, T cells produced this way can only be expanded in vitro for a few weeks before they become senescent, thus, limiting the number of microbial and tumor antigen-specific T cells, TCR-T cells or CAR-T cells that can be produced from each patient or healthy donor.

Recent reports suggest that factors that promote the survival of CAR-T cells by gene engineering is positively associated with better therapeutic effect (Hurton et al., 2016). Therefore, strategies that increase the lifespan of normal and/or genetically altered T cells and preserve their proliferative, cytokine production, and cytotoxic functions would significantly decrease the time to produce and the cost of adoptive T cell therapy approaches while potentially increasing their efficacy. While the cytotoxic T cell line, TALL-104 (U.S. Pat. No. 5,272,082) and the NK cell line, NK-92 (U.S. Patent Publication No. US20020068044), can proliferate indefinitely and have cytotoxic activity they were established from T cell and NK cell leukemias, respectively. Thus, these cell lines contain mutations and other genetic alterations and are unsafe for therapeutic use in humans. Thus, there is an unmet need for strategies that achieve these goals for increasing the lifespan of normal T cells.

### SUMMARY

In one embodiment, the present disclosure provides a composition comprising immune cells, including at least T cells or NK cells, that are engineered to have an increased lifespan compared to immune cells that have not been so engineered. Such cells may be referred to herein as infinite cells. In particular embodiments, methods and compositions concern immune cells having expression, including heterologous expression, of B-cell lymphoma 6 (BCL6) and a pro-survival gene or anti-apoptotic gene or cell survival-promoting gene. As used herein, the pro-survival gene refers to a nucleic acid polymer that can exert anti-apoptosis function or promote survival by any mechanism. The nucleic acid polymers that can exert anti-apoptosis function may be one or more of Bcl2 family genes such as BCL-xL (also known as BCL2L1 gene), BCL-2, MCL1, BCL2L2 (Bcl-w), BCL2A1 (Bfl-1), BCL2L10 (BCL-B), etc. The nucleic acid polymers that can exert anti-apoptosis function may be one or more of inhibitor of apoptosis (IAP) family genes, such as XIAP, BIRC2 (C-IAPl), BIRC3 (C-IAP2), NAIP, BIRC5 (survivin), etc. The nucleic acid that can exert anti-apoptosis function may be able to inhibit or knock out expression of one or more caspases that play a role in apoptosis, such as Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14. Nucleic acid polymers for knockdown or knock-out could be an shRNA expression cassette, or these caspase genes can also be knocked out by gene editing method (CRISPR, TALEN, Zinc finger method, etc.). The nucleic acid polymers that can exert anti-apoptosis function may be able to inhibit or knock out expression of one or more pro-apoptotic genes, such as BCL2L11 (BIM), BBC3 (PUMA), PMAIP1 (NOXA), BIK, BMF, BAD, HRK, BID, BAX, BAK1, BOK, etc. The nucleic acid polymers that can exert anti-apoptosis function may be have an anti-apoptotic effect, such as IGF1, HSPA4 (Hsp70), HSPB1 (Hsp27), CLAR (cFLIP), BNIP3, FADD, AKT, and NF-κB, RAF1, MAP2K1 (MEK1), RPS6KA1 (p90Rsk), JUN, C-Jun, BNIP2, BAG1, HSPA9, HSP90B1, miRNA21, miR-106b-25, miR-206, miR-221/222, miR-17-92, miR-133, miR-143, miR-145, miR-155, miR-330, etc.

In particular embodiments, the cells encompassed herein are able to constitutively produce large amounts of IL-4 (for example, greater than 1000 pg/mL in in vitro culture when incubated at a cell concentration of 10,000 cells/mL) in the absence of external stimulus, and such cells may be utilized for clinical application, such as for treatment of various inflammatory disorders, including autoimmune diseases, graft-versus-host disease, certain types of infections associated with cytokine release syndrome, toxicities associated with CAR T-cell and other adoptive T-cell therapies, inflammatory bowel disorders, immune-related adverse events associated with various immunotherapies, hemophagocytic lymphohistiocytosis, periodic fever syndromes, etc., as IL-4 can suppress inflammation induced by T cells, macrophages, and other immune cells.

In some aspects, the cell survival-promoting gene is an anti-apoptotic B-cell lymphoma 2 (BCL-2) family gene. In certain aspects, the anti-apoptotic BCL-2 family gene is BCL2L1 (Bcl-xL), BCL-2, MCL1, BCL2L2 (Bcl-w), BCL2A1 (Bfl-1), BCL2L10 (BCL-B), or a combination thereof. In particular aspects, the anti-apoptotic BCL-2 family gene is Bcl-xL.

In further aspects, the T cells or NK cells are further engineered to express IL-2 and/or IL-15.

In certain aspects, the T cell or NK cells are derived from a healthy donor (e.g., donor that has not been diagnosed with cancer). In other aspects, the T cell or NK cells are derived from a patient. In particular aspects, the donor is human.

In specific aspects, the T cells comprise CD4+ T cells, CD8+ T cells, iNKT cells, NKT cells, γδ T cells, regulatory T cells, innate lymphoid cells, or a combination thereof. In some aspects, the T cells comprise CD8 and/or γδ T cells. the T cells are naïve T cells, effector T cells, memory T cells, stem cell memory T cells, terminally differentiated T cells, or a combination thereof. In certain aspects, the T cells are TCR αβ cells or TCR γδ T cells. In some aspects, the composition is free of or essentially free of follicular helper (Tfh) T cells. In some aspects, the composition of the immune cells are T cells that are Th1/Tc1, Th2/Tc2, Th9/Tc9, Th17/Tc17, Tfh, Th22, Tc22, or a combination thereof. In particular aspects, the T cells express IFNγ, granzyme B, perforin, or a combination thereof.

In certain aspects, the T cells or NK cells are virus-specific or tumor antigen-specific. In some aspects, the T cells or NK cells are further engineered to express one or more CARs and/or one or more TCRs. In some aspects, the CAR or TCR comprises a CD4, CD5, CD7, CD10, CD19, CD20, CD22, CD30, CD79a, CD79b, SLAM-F7, CD123, CD70, CD72, CD33, CD38, CD80, CD86, CD138, CLL-1, FLT3, ROR-1, TACI, TRBC1, MUC1, PD-L1, CD117, FR□, LeY, HER2, IL13Rα2, DLL3, DR5, FAP, LMP1, MAGE-A1, MAGE-A4, MG7, MUC16, PMEL, ROR2, VEGFR2, AFP, EphA2, PSCA, EPCAM, EGFR, PSMA, EGFRvIII, GPC3, CEA, GD2, NY-ESO-1, TCL1, mesothelin, or BAFF-R antigen binding region. In particular aspects, the CAR comprises a CD19 antigen binding region.

In certain aspects, the composition comprises at least 50 million, 100 million, 200 million, 500 million, 750 million, 1 billion, 2 billion, 3 billion, 4 billion, 5 billion, 6 billion, 7 billion, 8 billion, 9 billion, or 10 billion immune cells, including T cells, innate lymphoid cells, NK cells, or a mixture thereof.

In additional aspects, the immune cells comprise at least one safety switch. In some aspects, the safety switch is truncated EGFR (for example an EGFR lacking domains 1 and 2). In some aspects, the immune cells (T cells, innate lymphoid cells, and/or NK cells) express IL-2, IL-15, other growth or differentiation factors, or a combination thereof.

In some aspects, the cells maintain a proliferation rate for at least 3 months, 4 months, 5 months, 6 months, 7 months, 8 months, 9 months, 10 months, 11 months, 12 months, or any range therebetween. In certain aspects, the immune cells have enhanced antitumor cytotoxicity, in vivo proliferation, in vivo persistence, and/or improved function.

In another embodiment, there is provided a method for producing T cells, innate lymphoid cells, or NK cells of the present embodiments comprising introducing a vector encoding BCL6 and a cell survival-promoting gene to said cells. In some aspects, the cell survival-promoting gene is an anti-apoptotic B-cell lymphoma 2 (BCL-2) family gene. In some aspects, the anti-apoptotic BCL-2 family gene is BCL2L1 (Bcl-xL), BCL-2, MCL1, BCL2L2 (Bcl-w), BCL2A1 (Bfl-1), BCL2L10 (BCL-B). In particular aspects, the anti-apoptotic BCL-2 family gene is Bcl-xL. In certain aspects, the vector links BCL6 and Bcl-xL with a 2A sequence. In specific aspects, the 2A sequence is a T2A sequence.

In some aspects, the vector is a lentiviral vector. In certain aspects, introducing comprises transducing the cells with the lentiviral vector in the presence of IL-2 and/or other growth factor(s). In certain aspects, IL-2 is at a concentration of 10 IU/mL to 1000 IU/mL, such as 10-50 IU/mL, 50-75 IU/mL, 75-100 IU/mL, 100-250 IU/mL, 250-500 IU/mL, 500-750 IU/mL, or 750-1000 IU/mL. In particular aspects, IL-2 is at a concentration of 100, 200, 300, 400, or 500 IU/mL.

In additional aspects, the method further comprises activating the T cells with CD3 and CD28. In some aspects, the method further comprises culturing the cells in the presence of IL-2 and/or IL-15. In certain aspects, the IL-2 and/or IL-15 are present at a concentration of 10 ng/mL, 25 ng/mL, 50 ng/mL, 75 ng/mL, 100 ng/mL, 150 ng/mL, or 200 ng/mL. In some aspects, the cells are cultured for at least 3, 4, 5, 6, 7, 8, 9, 10, 11, 12 months (or any range therebetween) with essentially no decrease in rate of proliferation.

In further aspects, the method further comprises sorting for a T cell subset. In particular aspects, the T cell subset comprises CD4+ T cells, CD8+ T cells, and/or γδ T cells.

Embodiments include a composition comprising a population of cells of the present embodiments (e.g., immune cells engineered to express B-cell lymphoma 6 (BCL6) and a cell survival-promoting gene) for the treatment of an immune-related disorder, infectious disease, and/or cancer.

Embodiments concern a method of treating a disease or disorder in a subject comprising administering an effective amount of immune cells of the present embodiments (e.g., immune cells engineered to express B-cell lymphoma 6 (BCL6) and a cell survival-promoting gene) to the subject.

In some aspects, the disease or disorder is an infectious disease, cancer, and/or immune-related disorder. In certain aspects, the immune-related disorder is an autoimmune disorder, graft versus host disease, allograft rejection, or other inflammatory condition. In some aspects, the immune cells are allogeneic. In particular aspects, the immune-related disorder is a cancer. For example, the cancer is a solid cancer or a hematologic malignancy.

In additional aspects, the method further comprises administering at least a second therapeutic agent. In some aspects, the at least a second therapeutic agent comprises chemotherapy, immunotherapy, surgery, radiotherapy, drug therapy, hormone therapy, biotherapy, or a combination thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G: (FIG. 1A) Map of a lentiviral vector containing human PGK promoter driven BCL6-T2A-BCL-xL genes. (FIG. 1B) Graph illustrating the proliferation rate of infinite T cell lines. The upper left panel shows the growth curves of In1-L4a (Infinite CD3 T cells) and Ie1-L4aJ3 (Infinite CD8 CAR-T) in the presence of 400 IU/mL of IL-2 at month 2. The upper right panel shows growth curves of infinite CD8 CAR T cells (Ie1-L4aJ3) in the presence of 100 ng/mL of IL-15, IL-7 and IL-21 or no cytokine. The data show that infinite T cells grow in the presence of IL-15 but not IL-7, IL-21, or no cytokine. The lower left and lower right panels show that infinite T cells, including CD4 infinite αβ T cells, CD8 infinite αβ T cells (Ie1-L4a), CD8 infinite αβ CAR-T cells (Ie1-L4aJ3), infinite γδ T cells (Igd1-L4a), and infinite γδ CAR-T cells (Igd1-L4aJ3) continue to proliferate in vitro in the presence of IL-2 at month 5. (FIG. 1C) Graph illustrating the phenotype of infinite T cell line In1-L4a as determined by expression of CD3, CD4, CD8, CD16, CD56, TCRαβ, and TCRγδ. (FIG. 1D) Graph illustrating the phenotype of sorted γδ T cells using an anti-TCRγδ antibody. The expression of TCRγδ, TCRαβ, and CD16 on these cells is shown. (FIG. 1E) Graph illustrating the major subset of sorted γδ T cells using anti-TCRγ9 and anti-TCRδ2 antibodies. The majority of infinite γδ T cells are positive for TCR γ962. (FIG. 1F) Graph illustrating the phenotype of infinite T cells at month 4. The majority of them are effector and central memory T cells which express predominantly IFNγ, granzyme B, and perforin. (FIG. 1G) Graph illustrating the expression of various co-inhibitory receptors on infinite CAR-T cells.

FIGS. 2A-2E: (FIG. 2A) Map of a lentiviral vector pJ3 which contains an anti-CD19 CAR and truncated human EGFR expression cassette. (FIG. 2B) Graph demonstrating the CAR positive percentage of Ie1-L4aJ3 (Infinite CD8 CART) and In1-L4aJ3 (Infinite CD3 CART), which were transduced by a lentiviral vector pJ3. The CAR positive percentage was determined by flow cytometry using an FITC labelled human CD19 protein or anti-EGFR antibody 10 days after transduction. (FIG. 2C) Graph illustrating the percentage of CAR positive cells of In1-L4aJ3 (Infinite CD3 CART). The tEGFR was stained with AF647-labeled cetuximab, the anti-CD19 CAR was stained with a FITC labelled recombinant human CD19 protein. (FIG. 2D) Graph illustrating the percentage of CAR positive cells of In1-L4aJ3 (Infinite CD3 CART) before and after sorting. The tEGFR was stained with AF647-Cetuximab, the anti-CD19 CAR was stained with a FITC labelled recombinant human CD19 protein.

FIG. 3: Graph illustrating the in vitro cytotoxicity of Ie1-L4aJ3 (Infinite CD8 CART) against the Raji and Nalm6 cells at an effector:target (E:T) ratio of 0.2:1 and 1:1 ratio in a 12-well plate. The Ie1-L4aJ3 (Infinite CD8 CART) cells or the control Ie1-L4a (Infinite CD8 T cells without CAR) cells were co-cultured with Raji or Nalm6 cells for 5 days. The percentage of tumor cells in the co-cultures on days 0, 1, 3, and 5 are shown.

FIG. 4: Graph illustrating the in vitro cytotoxicity of infinite T cells after expansion for 4 months. Ie1-L4a (Infinite CD8 T cells), Ie1-L4aJ3 (Infinite CD8 CART), Igd1-L4a (infinite gamma/delta T cells), or Igd1-L4aJ3 (infinite γδ CAR-T cells, CAR-T percentage is >90%) cells were co-cultured with Daudi or Nalm6 cells for 7 days at an effector:target (E:T) ratio of 3:1 in a 12-well plate in the presence of IL-15. The percentage of tumor cells in the co-cultures on days 0, 1, 2, 4 and 7 are shown. These results suggest that 1) CD8 infinite CAR-T and γδ infinite CAR-T cells maintained the specific cytotoxicity even after long term in vitro culture and expansion and 2), γδ infinite T cells without CAR but with endogenous γ962 TCR or with other TCRs can induce lysis of certain types of tumor cells likely mediated by the γδ TCR. For example, Daudi cells can be killed by γδ infinite T cells without CAR, whereas Nalm-6 can only be killed by γδ infinite T cells transduced with CAR. In addition to some lymphoma tumor cells, some myeloma cell lines and other cancer cell lines are also known to be killed by γδ T cells.

FIGS. 5A-5C: (FIG. 5A) Growth rate of infinite T cells (CD4+CD8 or CD8) with or without anti-CD19 CAR in the presence of IL-2. (FIG. 5B) Infinite T cells have a mixture of both CD4 and CD8 T cells (left panel) and can be sorted to high purity as shown for CD8 infinite T cells (right panel). (FIG. 5C) Infinite T cells in culture for 6 months were then incubated without IL-2 (shown) or IL-15 (not shown). Cell number declined rapidly within 6 days suggesting that there was no evidence of autonomous growth or malignant transformation of the infinite T cells even after long-term in vitro culture.

FIGS. 6A-6B: (FIG. 6A) Telomerase activity was determined in infinite T cells or peripheral blood mononuclear cells (PBMC) using TRAPeze telomerase activity detection kit as per manufacturer's instructions. (FIG. 6B) Genes related to telomerase activity shown as heatmap in infinite T cells or corresponding PBMC samples as determined by RNAseq analysis. These results suggest that infinite T cells have a very high telomerase activity.

FIGS. 7A-7D. (FIG. 7A) Infinite T cells with or without anti-CD19 CAR or CAR T cells generated by conventional methods from peripheral blood T cells were labeled with CellTrace FarRed and Daudi tumor cells were labeled with CellTrace Violet and co-cultured at Effector:Target ratio of 1:1. Percent live tumor cells (lower right gate) was determined after 3, 5, and 7 days. The absolute numbers of live tumor cells were also calculated using CountBright Absolute counting beads (ThermoFisher Scientific) by flow cytometry and the results were consistent with the percentage of live tumor cells shown. (FIG. 7B) Infinite T cells with or without anti-CD19 CAR were co-cultured 1:1 with NALM-6 B cell leukemia cells. Degranulation was determined by CD107a staining after 6 h. These results suggest that infinite T cells expressing CAR are highly cytotoxic and degranulate in response to B-cell tumors. (FIG. 7C and FIG. 7D) Phenotype of anti-CD19 infinite CAR T cells was determined for the markers shown by flow cytometry. Anti-CD19 CAR expression was determined by staining with fluorescently labeled recombinant human CD19-Fc protein. The results show that infinite T cells do not express high levels of conventional markers of exhaustion such as CTLA-4, PD-1, TIM-3, CD160, or 2B4 (CD244).

FIGS. 8A-8D: Genes or gene signatures related to T-cell subsets (FIG. 8A), exhaustion markers (FIG. 8B), chemokine receptors (FIG. 8C), and senescence markers (FIG. 8D) shown as heatmap in infinite T cells or corresponding PBMC samples as determined by RNAseq analysis.

FIGS. 9A-9C: Genes related to chemokine expression (FIG. 9A), cytokine expression (FIG. 9B), and cytokine receptors (FIG. 9C) shown as heatmap in infinite T cells or corresponding PBMC samples as determined by RNAseq analysis.

FIGS. 10A-10C: (FIG. 10A) Infinite T cells or CAR-transduced T cells were thawed and expression of anti-CD19 CAR was determined by anti-EGFR antibody staining. (FIG. 10B) Growth rate of anti-CD19 infinite CAR T cells after thawing and in vitro culture with IL-2. Number of cells in culture on different days is shown. (FIG. 10C) Cytotoxic activity of cells thawed in A was determined as described under FIG. 7A after 4 days of 1:1 co-culture between infinite T cells and NALM-6 tumor cells. Gate shows percent live tumor cells.

FIG. 11: Phenotype of infinite γδ T cells (bottom) was determined for the markers shown by flow cytometry and compared with corresponding γδ T cells from healthy donor PBMC (top). The results show that infinite γδ T cells do not express high levels of conventional markers of exhaustion.

FIG. 12: Luciferase-labeled infinite T cells were injected intraperitoneally (i.p.) with or without IL-15 injection on days 1&3. T cell numbers were imaged by bioluminescence imaging (BLI). The results show that IL-15 promotes growth and expansion of infinite T cells in vivo.

FIG. 13: Luciferase-labeled NALM-6 cells were injected into NSG mice along with infinite T cells with or without anti-CD19 CAR+/− IL-15. Antitumor efficacy was determined by BLI (left) and survival (right). The results show that anti-CD19 infinite CAR T cells have antitumor efficacy in vivo.

FIG. 14: Antigen-specific infinite T cells. Infinite T cells from an HLA-A2⁺ donor were tested for specificity against infectious disease and tumor-associated antigens using HLA-A2 tetramers with known CD8 T-cell epitopes. Data show presence of antigenspecific T cells in infinite T cells that recognized microbial and tumor-associated antigens via their endogenous TCR.

FIG. 15: Generation of EBV-specific infinite T cells. Healthy donor peripheral blood mononuclear cells from an HLA-A2+ donor were stimulated with a pool for HLA-A2-binding EBV peptides on day 0 and CD137 positive T cells were sorted by flow cytometry after 24 hours and used for generation of infinite T cells as previously described by transducing BCL6 and Bcl-xL. After 7 weeks of culture, tetramer positive cells were enriched by magnetic beads, then the enriched cells were cultured for another 6 more weeks and stained for CD8 and BMLF1-HLA-A2 tetramer specific against an HLA-A2-binding peptide (GLCTLVAML) derived from EBV-BMLF1 protein. These results suggest that an enriched population of microbial or tumor antigen-specific infinite CD4 or CD8 T cells may be generated using the method described.

FIG. 16: Infinite αβ or γδ T cells were generated with BCL6 and BCL2L1 genes under the control of the Tet-off safety switch. Growth rate of infinite T cells with IL-2 in the absence (Left) or presence of doxycycline (Dox) (Right) at 1 μg/mL is shown. The results suggest that infinite T cells maintain their growth rate in the absence of doxycycline but stopped proliferating and underwent gradual cell death in the presence of doxycycline. A similar tet-off safety switch can also be used for control of IL-2 or IL-15 cytokine genes incorporated into infinite T cells.

FIG. 17: Infinite T cells with tet-off safety switch were cultured with IL-2 in the presence or absence of increasing concentrations of doxycycline (Dox) and cells in culture were imaged by light microscopy. Cells were also stained to assess CD25 expression by flow cytometry after 2 weeks. By light microscopy imaging, the infinite T cells were found to gradual decrease in size along with decrease in proliferation clusters with increasing concentrations of doxycycline. In addition, the CD25 expression decreased markedly in the presence of doxycycline.

FIG. 18: Infinite T cells with tet-off safety switch were cultured with IL-2 in the presence or absence of doxycycline (Dox) at 1 μg/mL and cells were stained after 2 weeks to assess for the indicated surface markers by flow cytometry. PD-1 expression increased markedly in the presence of doxycycline.

FIG. 19: Cytokine production by infinite T cells. Infinite T cells (CD8+) with or without anti-CD19 CAR expression were co-cultured with NALM-6 tumor cells at an effector:target ratio of 5:1. After 3 days, cytokine levels were measured in the supernatants. Data is representative of results from infinite T cells derived from three different healthy donors. The results show that infinite T cells with anti-CD19 CAR but not without predominantly produced significant amounts of IL-2, GM-CSF, IFNγ, IL-5, and IL-17 in response to NALM-6 tumor cells. Production of TNFα, IL-4, IL-6, IL-10, or IL-13 by anti-CD19 infinite CAR T cells in response to tumor cells was minimal or not significantly different from infinite T cells without CAR expression. However, we observed that infinite T cells with or without CAR expression produced large amounts of IL-4 exceeding 10,000 pg/mL in the presence or absence of tumor cells (FIG. 19 and data not shown).

FIG. 20: Lysis of infinite CAR T cells by cetuximab via antibody-dependent cell-mediated cytotoxicity (ADCC). Infinite T cells expressing anti-CD19 CAR and tEGFR were labeled with CFSE and co-cultured in duplicates with or without NK cells derived from healthy donor at the indicated effector:target ratios in the presence of cetuximab or rituximab at 5 μg/mL. After 5 hours, the absolute number of infinite T cells were determined in each well by flow cytometry using counting beads and the percent decrease in infinite T cell number compared to T cells alone was calculated and shown in the graph. The percent decrease in T cells with either cetuximab or rituximab in the absence of NK cells was <5%.

FIGS. 21A-21C: Generation of infinite T cells with either BCL6 and BCL2L1 genes or BCL6 and BIRC5 (survivin) genes and Tet-off safety switch and IL-15. (FIG. 21A) Design of lentiviral constructs with either BCL6 and BCL2L1 genes or BCL6 and BIRC5 genes, Tet-off safety switch, and IL-15 gene. (FIG. 21B) Human T cells were lentivirally transduced with constructs shown in panel A and cultured in the presence of IL-2. The growth rate of the T cells generated by the two approaches during in vitro culture under similar conditions was determined after 12 weeks. (FIG. 21C) Infinite T cells were generated from two donors with the lentiviral construct containing BCL6 and BCL2L1 genes shown in panel A and cultured with IL-2 in the presence or absence of doxycycline at 1 μg/mL. The cells grew at an exponential rate in the absence of doxycycline but stopped proliferating and underwent gradual cell death in the presence of doxycycline.

FIG. 22: One example of a construct (L5x(MSCV-BCL6-P2A-BCL-xl-T2A-rtTA)) including BCL6 with Bcl-xl. The structure includes at least wild-type BCL-6 separated from BCL-xL by a P2A element, and BCL-xL is separated from rtTA (Tet on transactivator) by a T2A element.

FIG. 23: Illustration of examples of specific embodiments of constructs including at least for expression of BCL6. Some embodiments include shRNAs of any kind, including against Caspase 9 or BAK, as examples.

### DESCRIPTION OF ILLUSTRATIVE EMBODIMENTS

Ectopic expression of human telomerase reverse transcriptase (hTERT) gene was previously reported to immortalize normal T cells (Hooijberg et al., 2000). However, it has been observed that overexpression of hTERT alone is not sufficient for T lymphocyte immortalization. In fact, T cells generated by this approach stop proliferating after some time (Migliaccio et al., 2000). The present studies considered that expression of BCL6 in normal NK or T cells may stop their differentiation and that the expression of cell survival promoting genes such as anti-apoptotic BCL-2 family genes, like BCL2L1 encoding Bcl-xL protein, might significantly extend their lifespan, possibly immortalizing them while maintaining their basic functions.

Embodiments of the present disclosure concern compositions, production, and use of cells that have a significantly increased lifespan compared to cells lacking the modification(s) encompassed herein. In specific embodiments, the cells encode heterologous BCL6 and one or more pro-survival genes (or anti-apoptotic gene or cell survival-promoting gene), including any gene whose gene product has anti-apoptotic function. As examples, the pro-survival gene may be any BCL-2 family gene, including BCL-xL, BCL-2, MCL-1, or Survivin, as examples only. Additionally, or alternatively, the cells have inhibition of expression or knock out of expression of one or more caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, or a combination thereof). In such an example, the DNA fragments for knockdown or knock-out of one or more caspase genes could be an shRNA expression cassette. These caspase genes can also be knocked out by gene editing method (CRISPR, TALEN, Zinc finger method, etc.). Therefore, in specific embodiments the immune cells comprise a caspase knock-out in addition to overexpression of BCL6 or in addition of heterologous BCL6 to generate infinite immune cells. The cells may have one or more pro-survival genes (or anti-apoptotic gene or cell survival-promoting gene) and may also have knockdown or knock-out of one or more caspase genes, in specific cases.

The present disclosure provides, in certain embodiments, methods for the production of an unlimited number of infinite immune cells that have a significantly increased lifespan and can be grown into large numbers rapidly, such as for adoptive immunotherapy. The present methods provide infinite immune cells with the ability to indefinitely expand by a one-time transduction, in at least some cases. The present methods are very inexpensive and can generate unlimited number of immune cells in a short period of time (for example, one month or more).

This platform and system encompassed herein can be used to generate infinite immune cells, such as infinite T cells including both TCR αβ and TCR γδ T cells. This approach provides an unlimited source of human T cells that can be used as such or can be genetically engineered further to produce desired cells, including off-the-shelf chimeric antigen receptor (CAR) T cells or T cell receptor (TCR)-transduced T cells. In specific embodiments, the cells are utilized to treat or prevent cancer and other diseases including infectious and inflammatory disorders. As examples, the system can be used to treat cancer, infectious diseases, and/or inflammatory diseases. Specific examples include B-cell lymphoma, CMV infectious disease, EBV infectious disease, autoimmune disorders, graft-versus-host disease, or a combination thereof.

As one example, the studies encompassed herein showed that transduction of anti-CD19 CAR into the infinite T cells generated ‘anti-CD19 infinite CAR T cells’ (CD19 inCART) and redirected their specificity against human B cell tumors. The CD19 infinite CAR T cells can serve as a source to generate unlimited number of antigen receptor-modified T cells (such as CAR T cells) after just one transduction and exhibited significant cytotoxicity against human B cell lymphoma cell lines. The present disclosure provides an off-the-shelf immune cell therapy platform and system that can produce an unlimited number of immune cells and can dramatically reduce the cost and production time of adoptive immune cell therapies by streamlining the manufacturing process. Particular embodiments allow for the generation of infinite cells by expressing BCL6 and one or more pro-survival genes (or anti-apoptotic genes or cell survival-promoting genes) that acts as an off-the-shelf cell for further manipulation for adoptive cell therapy, such as further manipulation by incorporating an engineered antigen receptor of interest (for example, tailored to a specific cancer). The off-the-shelf cells may also already include one or more safety switches (including, e.g., an inducible system as well as an elimination gene, such as truncated EGFR (as one example, lacking domain 1 and/or domain 2) and/or one or more suicides genes and/or one or more cytokines, or any of these may be added later in a step to tailor the cells to have desired properties.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more. The terms “about”, “substantially” and “approximately” mean, in general, the stated value plus or minus 5%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B cell or a T cell response) against an antigen that is part of the normal host (that is, an autoantigen), with consequent injury to tissues. An autoantigen may be derived from a host cell, or may be derived from a commensal organism such as the microorganisms (known as commensal organisms) that normally colonize mucosal surfaces.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

### II. INFINITE IMMUNE CELLS

Certain embodiments of the present disclosure concern immune cells that are engineered to express one or more genes. The expression of the one or more genes directly or indirectly results in the increased lifespan of the cells compared to cells that lack the expression of the one or more genes. In particular embodiments, the cells are manipulated to express the one or more genes, including one or more heterologous genes. In other cases, the cells are manipulated to have upregulation of expression of the one or more genes that are endogenous to the cells, such as through manipulation of one or more regulatory elements of the one or more endogenous genes to the cells.

In particular embodiments, immune cells are manipulated to express BCL6 and one or more pro-survival genes or anti-apoptotic genes or cell survival-promoting genes (and there may or may not be overlap in a gene that is classified as pro-survival or anti-apoptotic or cell survival-promoting). As used herein, the pro-survival gene refers to a nucleic acid polymer that can exert anti-apoptosis function or promote survival by any mechanism. The nucleic acid polymer that can exert anti-apoptosis function may be one or more of Bcl2 family genes such as BCL-xL, BCL-2, MCL-1, Bcl-w, Bfl-1, BCL-B, etc. The nucleic acid polymer that can exert anti-apoptosis function may be one or more of inhibitor of apoptosis (IAP) family genes, such as XIAP, c-IAPl, C-IAP2, NAIP, and Survivin, etc. The nucleic acid polymer that can exert anti-apoptosis function may be able to inhibit or knock out expression of one or more caspases that play a role in apoptosis, such as Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14. Nucleic acid polymers for knockdown or knock-out could be an shRNA expression cassette, or these caspase genes can also be knocked out by gene editing method (CRISPR, TALEN, Zinc finger method, etc.). The nucleic acid polymer that can exert anti-apoptosis function may be able to inhibit or knock out expression of one or more pro-apoptotic genes, such as BIM, Puma, Noxa, Bik, Bmf, Bad, Hrk, Bid, BAX, BAK, BOK, etc. The nucleic acid polymer that can exert anti-apoptosis function may have an anti-apoptotic effect, such as insulin-like growth factor (IGF-1), Hsp70, Hsp27, cFLIP, BNIP3, FADD, Akt, and NF-κB, Raf-1 and MEK1, p90Rsk, C-Jun, BNIP2, BAG1, HSPA9, HSP90B1, miRNA21, miR-106b-25, miR-206, miR-221/222, miR-17-92, miR-133, miR-143, miR-145, miR-155, miR-330, etc.

Infinite T cells may be generated with either wild type or mutant BCL6. The inventors determined that infinite T cells could be generated with either wildtype BCL6 or mutant BCL6 with a single particular nucleotide difference—the codon of the amino acid at position 395 in wild type BCL6 is CCT (encoding Proline/P) and the codon of the amino acid at position 395 in mutant BCL6 is CTT (encoding Leucine/L). The nucleotide and amino acid sequences for the two BCL6 genes are shown below (with the point of mutation in the wildtype sequence being underlined).

The aa sequence of wildtype BCL6:

(SEQ ID NO: 1)
MASPADSCIQFTRHASDVLLNLNRLRSRDILTDVVIVVSREQFRAHKT
VLMACSGLFYSIFTDQLKCNLSVINLDPEINPEGFCILLDFMYTSRLN
LREGNIMAVMATAMYLQMEHVVDTCRKFIKASEAEMVSAIKPPREEFL
NSRMLMPQDIMAYRGREVVENNLPLRSAPGCESRAFAPSLYSGLSTPP
ASYSMYSHLPVSSLLFSDEEFRDVRMPVANPFPKERALPCDSARPVPG
EYSRPTLEVSPNVCHSNIYSPKETIPEEARSDMHYSVAEGLKPAAPSA
RNAPYFPCDKASKEEERPSSEDEIALHFEPPNAPLNRKGLVSPQSPQK
SDCQPNSPTESCSSKNACILQASGSPPAKSPTDPKACNWKKYKFIVLN
SLNQNAKPEGPEQAELGRLSPRAYTAPPACQPPMEPENLDLQSPTKLS
ASGEDSTIPQASRLNNIVNRSMTGSPRSSSESHSPLYMHPPKCTSCGS
QSPQHAEMCLHTAGPTFPEEMGETQSEYSDSSCENGAFFCNECDCRFS
EEASLKRHTLQTHSDKPYKCDRCQASFRYKGNLASHKTVHTGEKPYRC
NICGAQFNRPANLKTHTRIHSGEKPYKCETCGARFVQVAHLRAHVLIH
TGEKPYPCEICGTRFRHLQTLKSHLRIHTGEKPYHCEKCNLHFRHKSQ
LRLHLRQKHGAITNTKVQYRVSATDLPPELPKAC

The nucleotide sequence of wildtype BCL6 (with the codon for the point of mutation in the wildtype sequence being underlined):

(SEQ ID NO: 2)
ATGgcctcgccggctgacagctgtatccagttcacccgccatgccagt
gatgttcttctcaaccttaatcgtctccggagtcgagacatcttgact
gatgttgtcattgttgtgagccgtgagcagtttagagcccataaaacg
gtcctcatggcctgcagtggcctgttctatagcatctttacagaccag
ttgaaatgcaaccttagtgtgatcaatctagatcctgagatcaaccct
gagggattctgcatcctcctggacttcatgtacacatctcggctcaat
ttgcgggagggcaacatcatggctgtgatggccacggctatgtacctg
cagatggagcatgttgtggacacttgccggaagtttattaaggccagt
gaagcagagatggtttctgccatcaagcctcctcgtgaagagttcctc
aacagccggatgctgatgccccaagacatcatggcctatcggggtcgt
gaggtggtggagaacaacctgccactgaggagcgcccctgggtgtgag
agcagagcctttgcccccagcctgtacagtggcctgtccacaccgcca
gcctcttattccatgtacagccacctccctgtcagcagcctcctcttc
tccgatgaggagtttcgggatgtccggatgcctgtggccaaccccttc
cccaaggagcgggcactcccatgtgatagtgccaggccagtccctggt
gagtacagccggccgactttggaggtgtcccccaatgtgtgccacagc
aatatctattcacccaaggaaacaatcccagaagaggcacgaagtgat
atgcactacagtgtggctgagggcctcaaacctgctgccccctcagcc
cgaaatgccccctacttcccttgtgacaaggccagcaaagaagaagag
agaccctcctcggaagatgagattgccctgcatttcgagccccccaat
gcacccctgaaccggaagggtctggttagtccacagagcccccagaaa
tctgactgccagcccaactcgcccacagagtcctgcagcagtaagaat
gcctgcatcctccaggcttctggctcccctccagccaagagccccact
gaccccaaagcctgcaactggaagaaatacaagttcatcgtgctcaac
agcctcaaccagaatgccaaaccagaggggcCtgagcaggctgagctg
ggccgcctttccccacgagcctacacggccccacctgcctgccagcca
cccatggagcctgagaaccttgacctccagtccccaaccaagctgagt
gccagcggggaggactccaccatcccacaagccagccggctcaataac
atcgttaacaggtccatgacgggctctccccgcagcagcagcgagagc
cactcaccactctacatgcaccccccgaagtgcacgtcctgcggctct
cagtccccacagcatgcagagatgtgcctccacaccgctggccccacg
ttccctgaggagatgggagagacccagtctgagtactcagattctagc
tgtgagaacggggccttcttctgcaatgagtgtgactgccgcttctct
gaggaggcctcactcaagaggcacacgctgcagacccacagtgacaaa
ccctacaagtgtgaccgctgccaggcctccttccgctacaagggcaac
ctcgccagccacaagaccgtccataccggtgagaaaccctatcgttgc
aacatctgtggggcccagttcaaccggccagccaacctgaaaacccac
actcgaattcactctggagagaagccctacaaatgcgaaacctgcgga
gccagatttgtacaggtggcccacctccgtgcccatgtgcttatccac
actggtgagaagccctatccctgtgaaatctgtggcacccgtttccgg
caccttcagactctgaagagccacctgcgaatccacacaggagagaaa
ccttaccattgtgagaagtgtaacctgcatttccgtcacaaaagccag
ctgcgacttcacttgcgccagaagcatggcgccatcaccaacaccaag
gtgcaataccgcgtgtcagccactgacctgcctccggagctccccaaa
gcctgc

The aa sequence of mutant BCL6 (the leucine mutation is underlined):

(SEQ ID NO: 3)
MASPADSCIQFTRHASDVLLNLNRLRSRDILTDVVIVVSREQFRAHKT
VLMACSGLFYSIFTDQLKCNLSVINLDPEINPEGFCILLDFMYTSRLN
LREGNIMAVMATAMYLQMEHVVDTCRKFIKASEAEMVSAIKPPREEFL
NSRMLMPQDIMAYRGREVVENNLPLRSAPGCESRAFAPSLYSGLSTPP
ASYSMYSHLPVSSLLFSDEEFRDVRMPVANPFPKERALPCDSARPVPG
EYSRPTLEVSPNVCHSNIYSPKETIPEEARSDMHYSVAEGLKPAAPSA
RNAPYFPCDKASKEEERPSSEDEIALHFEPPNAPLNRKGLVSPQSPQK
SDCQPNSPTESCSSKNACILQASGSPPAKSPTDPKACNWKKYKFIVLN
SLNQNAKPEGLEQAELGRLSPRAYTAPPACQPPMEPENLDLQSPTKLS
ASGEDSTIPQASRLNNIVNRSMTGSPRSSSESHSPLYMHPPKCTSCGS
QSPQHAEMCLHTAGPTFPEEMGETQSEYSDSSCENGAFFCNECDCRFS
EEASLKRHTLQTHSDKPYKCDRCQASFRYKGNLASHKTVHTGEKPYRC
NICGAQFNRPANLKTHTRIHSGEKPYKCETCGARFVQVAHLRAHVLIH
TGEKPYPCEICGTRFRHLQTLKSHLRIHTGEKPYHCEKCNLHFRHKSQ
LRLHLRQKHGAITNTKVQYRVSATDLPPELPKAC

The nucleotide sequence of mutant BCL6 (the codon for leucine is underlined):

(SEQ ID NO: 4)
ATGgcctcgccggctgacagctgtatccagttcacccgccatgccagt
gatgttcttctcaaccttaatcgtctccggagtcgagacatcttgact
gatgttgtcattgttgtgagccgtgagcagtttagagcccataaaacg
gtcctcatggcctgcagtggcctgttctatagcatctttacagaccag
ttgaaatgcaaccttagtgtgatcaatctagatcctgagatcaaccct
gagggattctgcatcctcctggacttcatgtacacatctcggctcaat
ttgcgggagggcaacatcatggctgtgatggccacggctatgtacctg
cagatggagcatgttgtggacacttgccggaagtttattaaggccagt
gaagcagagatggtttctgccatcaagcctcctcgtgaagagttcctc
aacagccggatgctgatgccccaagacatcatggcctatcggggtcgt
gaggtggtggagaacaacctgccactgaggagcgcccctgggtgtgag
agcagagcctttgcccccagcctgtacagtggcctgtccacaccgcca
gcctcttattccatgtacagccacctccctgtcagcagcctcctcttc
tccgatgaggagtttcgggatgtccggatgcctgtggccaaccccttc
cccaaggagcgggcactcccatgtgatagtgccaggccagtccctggt
gagtacagccggccgactttggaggtgtcccccaatgtgtgccacagc
aatatctattcacccaaggaaacaatcccagaagaggcacgaagtgat
atgcactacagtgtggctgagggcctcaaacctgctgccccctcagcc
cgaaatgccccctacttcccttgtgacaaggccagcaaagaagaagag
agaccctcctcggaagatgagattgccctgcatttcgagccccccaat
gcacccctgaaccggaagggtctggttagtccacagagcccccagaaa
tctgactgccagcccaactcgcccacagagtcctgcagcagtaagaat
gcctgcatcctccaggcttctggctcccctccagccaagagccccact
gaccccaaagcctgcaactggaagaaatacaagttcatcgtgctcaac
agcctcaaccagaatgccaaaccagaggggcTtgagcaggctgagctg
ggccgcctttccccacgagcctacacggccccacctgcctgccagcca
cccatggagcctgagaaccttgacctccagtccccaaccaagctgagt
gccagcggggaggactccaccatcccacaagccagccggctcaataac
atcgttaacaggtccatgacgggctctccccgcagcagcagcgagagc
cactcaccactctacatgcaccccccgaagtgcacgtcctgcggctct
cagtccccacagcatgcagagatgtgcctccacaccgctggccccacg
ttccctgaggagatgggagagacccagtctgagtactcagattctagc
tgtgagaacggggccttcttctgcaatgagtgtgactgccgcttctct
gaggaggcctcactcaagaggcacacgctgcagacccacagtgacaaa
ccctacaagtgtgaccgctgccaggcctccttccgctacaagggcaac
ctcgccagccacaagaccgtccataccggtgagaaaccctatcgttgc
aacatctgtggggcccagttcaaccggccagccaacctgaaaacccac
actcgaattcactctggagagaagccctacaaatgcgaaacctgcgga
gccagatttgtacaggtggcccacctccgtgcccatgtgcttatccac
actggtgagaagccctatccctgtgaaatctgtggcacccgtttccgg
caccttcagactctgaagagccacctgcgaatccacacaggagagaaa
ccttaccattgtgagaagtgtaacctgcatttccgtcacaaaagccag
ctgcgacttcacttgcgccagaagcatggcgccatcaccaacaccaag
gtgcaataccgcgtgtcagccactgacctgcctccggagctccccaaa
gcctgc

The immune cells may be any kind of immune cells, including T cells (e.g., regulatory T cells, CD4⁺ T cells, CD8⁺ T cells, alpha beta T cells, gamma-delta T cells, or a mixture thereof), NK cells, invariant NKT cells, NKT cells, innate lymphoid cells, or a mixture thereof. The immune cells may be virus-specific, express a CAR, and/or express a TCR. In some embodiments, the cells are monocytes or granulocytes, e.g., myeloid cells, macrophages, neutrophils, dendritic cells (DCs), mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells. These immune cells may be used for therapy as a single cell type or as a combination of multiple immune cell types. In specific embodiments, the immune cells are CD3+, CD4+, CD8+, CD16+, or a mixture thereof.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, such as a partially or fully histocompatibility matched donor or fully histocompatibility mismatched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor may be allogeneic, provided the cells obtained are subject-compatible in that they can be introduced into the subject. Allogeneic donor cells are may or may not be human-leukocyte-antigen (HLA)-compatible.

A. T Cells

In some embodiments, the immune cells are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs or PBMCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and gamma/delta T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells or stem cell memory cells is carried out to increase efficacy, such as to improve long-term survival, expansion, and/or engraftment following administration, which in some aspects is particularly robust in such sub-populations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2) or other growth factors.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide or peptides binding to other MHC class I or class II molecules, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells or antigen-presenting cells expressing other HLA molecules. The in vitro-induced T-cells may also be expanded in the absence of antigen-presenting cells..

The autologous T cells can be modified to express a T cell growth or differentiation factor that promotes the growth, differentiation, and activation of the autologous T cells. Suitable T cell growth factors include, for example, interleukin (IL)-2, IL-7, IL-15, IL-18, IL-21, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

B. NK Cells

In some embodiments, the immune cells are natural killer (NK) cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16, CD56, and/or CD8 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, tissues, or umbilical cord blood by methods well known in the art.

C. NKT Cells

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids. They constitute only approximately 0.1% of all peripheral blood T cells. NKT cells are a subset of T cells that coexpress an αβ T-cell receptor, but also express a variety of molecular markers that are typically associated with NK cells, such as NK1.1. Invariant natural killer T (iNKT) cells express high levels of and are dependent on the transcriptional regulator promyelocytic leukemia zinc finger for their development. Currently, there are five major distinct iNKT cell subsets. These subset cells produce a different set of cytokines once activated. The subtypes iNKT1, iNKT2 and iNKT17 mirror Th cell subsets in cytokine production. In addition, there are subtypes specialized in T follicular helper-like function and IL-10 dependent regulatory functions.

D. Innate Lymphoid Cells

Innate lymphoid cells (ILCs) are a group of innate immune cells that are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. They play a role in protective immunity and the regulation of homeostasis and inflammation, so their dysregulation can lead to immune pathology such as allergy, bronchial asthma and autoimmune disease. ILCs can be divided based on the cytokines that they can produce, and the transcription factors that regulate their development and function.

### III. PRODUCTION OF INFINITE IMMUNE CELLS

In some aspects, the present disclosure provides methods to increase the lifespan of immune cells by over-expression of BCL6 and of one or more pro-survival genes or anti-apoptotic genes or cell survival-promoting genes (including one or more anti-apoptotic BCL-2 family genes, such as Bxl-xL). The gene expression may be achieved by conventional molecular biology methods, such as cloning the coding sequences of BCL6 and the anti-apoptotic BCL-2 family gene downstream to a constitutive or inducible promoter in one or more viral or non-viral vectors, and delivering the vector(s) into the immune cells. Alternatively, the gene expression may be achieved by using CRISPR or other transposases to specifically transcribe the mRNAs of BCL6 and the anti-apoptotic BCL-2 family gene (as one example) in the immune cells. The expression of BCL6 and/or the anti-apoptotic BCL-2 family member (such as Bcl-xL) may be regulatable, including may be constitutive or inducible means. In some cases, expression of BCL6 and/or the anti-apoptotic BCL-2 family member may have a first type of regulation of expression (such as constitutive) and expression of one or more other genes in the system, such as on the same or another vector(s), may be regulated in the same manner (e.g., constitutive) or differently (such as inducible). In specific cases, BCL6-BCL-xL is regulated by a tet-off regulatable mechanism or a tet-on regulatable mechanism.

In one exemplary method, the coding sequences of BCL6 and Bcl-xL genes (merely as examples) can be joined but separated by an element that allows for ultimate production of separate BCL6 and Bcl-xL molecules. For example, the coding sequences of BCL6 and Bcl-xL genes can be joined but separated by a T2A sequence to generate one open reading frame that can express BCL6 and Bcl-xL genes simultaneously. This BCL6-T2A-Bcl-xL open reading frame may be cloned into a vector, such as a lentiviral vector. The immune cells, such as T cells, may then be transduced by the viral vector, such as in the presence of IL-2 and/or IL-15. This method can generate a T cell line referred to as ‘infinite T cells’ from healthy donor T cells, which can proliferate in the presence of recombinant human IL-2 and/or IL-15. In some cases, the cells are produced in the presence of IL-2 and/or IL-15 and the cells themselves also express heterologous IL-2 and/or IL-15, although in other cases just one of these parameters is utilized.

Examples of self-cleaving sequences are as follows:

T2A (GSG)
(SEQ ID NO: 5)
EGRGSLL TCGDVEENPGP
P2A (GSG)
(SEQ ID NO: 6)
ATNFSLLKQAGDVEENPGP
E2A (GSG)
(SEQ ID NO: 7)
QCTNYALLKLAGDVESNPGP
F2A (GSG)
(SEQ ID NO: 8)
VKQTLNFDLLKLAGDVESNPGP

In other cases, an IRES element is used instead of a 2A sequence.

In some embodiments, the cells are engineered to express a BCL6-2A-BCLxL sequence (SEQ ID NO:9) comprising human BCL6, a 2A self-cleaving peptide, and the BCL-xl coding sequence.

(SEQ ID NO: 9)
ATGgcctcgccggctgacagctgtatccagttcacccgccatgccagtgatgttcttctcaaccttaatc
gtctccggagtcgagacatcttgactgatgttgtcattgttgtgagccgtgagcagtttagagcccataaaacggtcctcatggcctgcagtgg
cctgttctatagcatctttacagaccagttgaaatgcaaccttagtgtgatcaatctagatcctgagatcaaccctgagggattctgcatcctcct
ggacttcatgtacacatctcggctcaatttgcgggagggcaacatcatggctgtgatggccacggctatgtacctgcagatggagcatgttgt
ggacacttgccggaagtttattaaggccagtgaagcagagatggtttctgccatcaagcctcctcgtgaagagttcctcaacagccggatgc
tgatgccccaagacatcatggcctatcggggtcgtgaggtggtggagaacaacctgccactgaggagcgcccctgggtgtgagagcaga
gcctttgcccccagcctgtacagtggcctgtccacaccgccagcctcttattccatgtacagccacctccctgtcagcagcctcctcttctccg
atgaggagtttcgggatgtccggatgcctgtggccaaccccttccccaaggagcgggcactcccatgtgatagtgccaggccagtccctg
gtgagtacagccggccgactttggaggtgtcccccaatgtgtgccacagcaatatctattcacccaaggaaacaatcccagaagaggcac
gaagtgatatgcactacagtgtggctgagggcctcaaacctgctgccccctcagcccgaaatgccccctacttcccttgtgacaaggccag
caaagaagaagagagaccctcctcggaagatgagattgccctgcatttcgagccccccaatgcacccctgaaccggaagggtctggttag
tccacagagcccccagaaatctgactgccagcccaactcgcccacagagtcctgcagcagtaagaatgcctgcatcctccaggcttctggc
tcccctccagccaagagccccactgaccccaaagcctgcaactggaagaaatacaagttcatcgtgctcaacagcctcaaccagaatgcc
aaaccagaggggcctgagcaggctgagctgggccgcctttccccacgagcctacacggccccacctgcctgccagccacccatggagc
ctgagaaccttgacctccagtccccaaccaagctgagtgccagcggggaggactccaccatcccacaagccagccggctcaataacatc
gttaacaggtccatgacgggctctccccgcagcagcagcgagagccactcaccactctacatgcaccccccgaagtgcacgtcctgcgg
ctctcagtccccacagcatgcagagatgtgcctccacaccgctggccccacgttccctgaggagatgggagagacccagtctgagtactc
agattctagctgtgagaacggggccttcttctgcaatgagtgtgactgccgcttctctgaggaggcctcactcaagaggcacacgctgcaga
cccacagtgacaaaccctacaagtgtgaccgctgccaggcctccttccgctacaagggcaacctcgccagccacaagaccgtccataccg
gtgagaaaccctatcgttgcaacatctgtggggcccagttcaaccggccagccaacctgaaaacccacactcgaattcactctggagagaa
gccctacaaatgcgaaacctgcggagccagatttgtacaggtggcccacctccgtgcccatgtgcttatccacactggtgagaagccctatc
cctgtgaaatctgtggcacccgtttccggcaccttcagactctgaagagccacctgcgaatccacacaggagagaaaccttaccattgtgag
aagtgtaacctgcatttccgtcacaaaagccagctgcgacttcacttgcgccagaagcatggcgccatcaccaacaccaaggtgcaatacc
gcgtgtcagccactgacctgcctccggagctccccaaagcctgcGGAAGCGGAGCTACTAACTTCAGCCTGCT
GAAGCAGGCTGGAGACGTGGAGGAGAACCCTGGACCTAGATCTGGAATGTCTCAGA
GCAACCGGGAGCTGGTGGTTGACTTTCTCTCCTACAAGCTTTCCCAGAAAGGATACA
GCTGGAGTCAGTTTAGTGATGTGGAAGAGAACAGGACTGAGGCCCCAGAAGGGACT
GAATCGGAGATGGAGACCCCCAGTGCCATCAATGGCAACCCATCCTGGCACCTGGC
AGACAGCCCCGCGGTGAATGGAGCCACTGGCCACAGCAGCAGTTTGGATGCCCGGG
AGGTGATCCCCATGGCAGCAGTAAAGCAAGCGCTGAGGGAGGCAGGCGACGAGTTT
GAACTGCGGTACCGGCGGGCATTCAGTGACCTGACATCCCAGCTCCACATCACCCCA
GGGACAGCATATCAGAGCTTTGAACAGGTAGTGAATGAACTCTTCCGGGATGGGGT
AAACTGGGGTCGCATTGTGGCCTTTTTCTCCTTCGGCGGGGCACTGTGCGTGGAAAG
CGTAGACAAGGAGATGCAGGTATTGGTGAGTCGGATCGCAGCTTGGATGGCCACTT
ACCTGAATGACCACCTAGAGCCTTGGATCCAGGAGAACGGCGGCTGGGATACTTTT
GTGGAACTCTATGGGAACAATGCAGCAGCCGAGAGCCGAAAGGGCCAGGAACGCTT
CAACCGCTGGTTCCTGACGGGCATGACTGTGGCCGGCGTGGTTCTGCTGGGCTCACT
CTTCAGTCGGAAAtgA-3

Another example of an expression construct comprising BCL6 and Bcl-xL is below, where the single underlined part is BCL6, the non-undelined part is P2A, the double-underlined part is BcL-xL:

(SEQ ID NO: 10)
ATGgcctcgccggctgacagctgtatccagttcacccgccatgccagtgatgttcttctcaaccttaatc
gtctccggagtcgagacatcttgactgatgttgtcattgttgtgagccgtgagcagtttagagcccataaaacggtcctcatggcctgcagtgg
cctgttctatagcatctttacagaccagttgaaatgcaaccttagtgtgatcaatctagatcctgagatcaaccctgagggattctgcatcctcct
ggacttcatgtacacatctcggctcaatttgcgggagggcaacatcatggctgtgatggccacggctatgtacctgcagatggagcatgttgt
ggacacttgccggaagtttattaaggccagtgaagcagagatggtttctgccatcaagcctcctcgtgaagagttcctcaacagccggatgc
tgatgccccaagacatcatggcctatcggggtcgtgaggtggtggagaacaacctgccactgaggagcgcccctgggtgtgagagcaga
gcctttgcccccagcctgtacagtggcctgtccacaccgccagcctcttattccatgtacagccacctccctgtcagcagcctcctcttctccg
atgaggagtttcgggatgtccggatgcctgtggccaaccccttccccaaggagcgggcactcccatgtgatagtgccaggccagtccctg
gtgagtacagccggccgactttggaggtgtcccccaatgtgtgccacagcaatatctattcacccaaggaaacaatcccagaagaggcac
gaagtgatatgcactacagtgtggctgagggcctcaaacctgctgccccctcagcccgaaatgccccctacttcccttgtgacaaggccag
caaagaagaagagagaccctcctcggaagatgagattgccctgcatttcgagccccccaatgcacccctgaaccggaagggtctggttag
tccacagagcccccagaaatctgactgccagcccaactcgcccacagagtcctgcagcagtaagaatgcctgcatcctccaggcttctggc
tcccctccagccaagagccccactgaccccaaagcctgcaactggaagaaatacaagttcatcgtgctcaacagcctcaaccagaatgcc
aaaccagaggggcctgagcaggctgagctgggccgcctttccccacgagcctacacggccccacctgcctgccagccacccatggagc
ctgagaaccttgacctccagtccccaaccaagctgagtgccagcggggaggactccaccatcccacaagccagccggctcaataacatc
gttaacaggtccatgacgggctctccccgcagcagcagcgagagccactcaccactctacatgcaccccccgaagtgcacgtcctgcgg
ctctcagtccccacagcatgcagagatgtgcctccacaccgctggccccacgttccctgaggagatgggagagacccagtctgagtactc
agattctagctgtgagaacggggccttcttctgcaatgagtgtgactgccgcttctctgaggaggcctcactcaagaggcacacgctgcaga
cccacagtgacaaaccctacaagtgtgaccgctgccaggcctccttccgctacaagggcaacctcgccagccacaagaccgtccataccg
gtgagaaaccctatcgttgcaacatctgtggggcccagttcaaccggccagccaacctgaaaacccacactcgaattcactctggagagaa
gccctacaaatgcgaaacctgcggagccagatttgtacaggtggcccacctccgtgcccatgtgcttatccacactggtgagaagccctatc
cctgtgaaatctgtggcacccgtttccggcaccttcagactctgaagagccacctgcgaatccacacaggagagaaaccttaccattgtgag
aagtgtaacctgcatttccgtcacaaaagccagctgcgacttcacttgcgccagaagcatggcgccatcaccaacaccaaggtgcaatacc
gcgtgtcagccactgacctgcctccggagctccccaaagcctgcGGAAGCGGAGCTACTAACTTCAGCCTGCT
GAAGCAGGCTGGAGACGTGGAGGAGAACCCTGGACCTAGATCTGGAATGTCTCAGA
GCAACCGGGAGCTGGTGGTTGACTTTCTCTCCTACAAGCTTTCCCAGAAAGGATACA
GCTGGAGTCAGTTTAGTGATGTGGAAGAGAACAGGACTGAGGCCCCAGAAGGGACT
GAATCGGAGATGGAGACCCCCAGTGCCATCAATGGCAACCCATCCTGGCACCTGGC
AGACAGCCCCGCGGTGAATGGAGCCACTGGCCACAGCAGCAGTTTGGATGCCCGGG
AGGTGATCCCCATGGCAGCAGTAAAGCAAGCGCTGAGGGAGGCAGGCGACGAGTTT
GAACTGCGGTACCGGCGGGCATTCAGTGACCTGACATCCCAGCTCCACATCACCCCA
GGGACAGCATATCAGAGCTTTGAACAGGTAGTGAATGAACTCTTCCGGGATGGGGT
AAACTGGGGTCGCATTGTGGCCTTTTTCTCCTTCGGCGGGGCACTGTGCGTGGAAAG
CGTAGACAAGGAGATGCAGGTATTGGTGAGTCGGATCGCAGCTTGGATGGCCACTT
ACCTGAATGACCACCTAGAGCCTTGGATCCAGGAGAACGGCGGCTGGGATACTTTT
GTGGAACTCTATGGGAACAATGCAGCAGCCGAGAGCCGAAAGGGCCAGGAACGCTT
CAACCGCTGGTTCCTGACGGGCATGACTGTGGCCGGCGTGGTTCTGCTGGGCTCACT
CTTCAGTCGGAAA

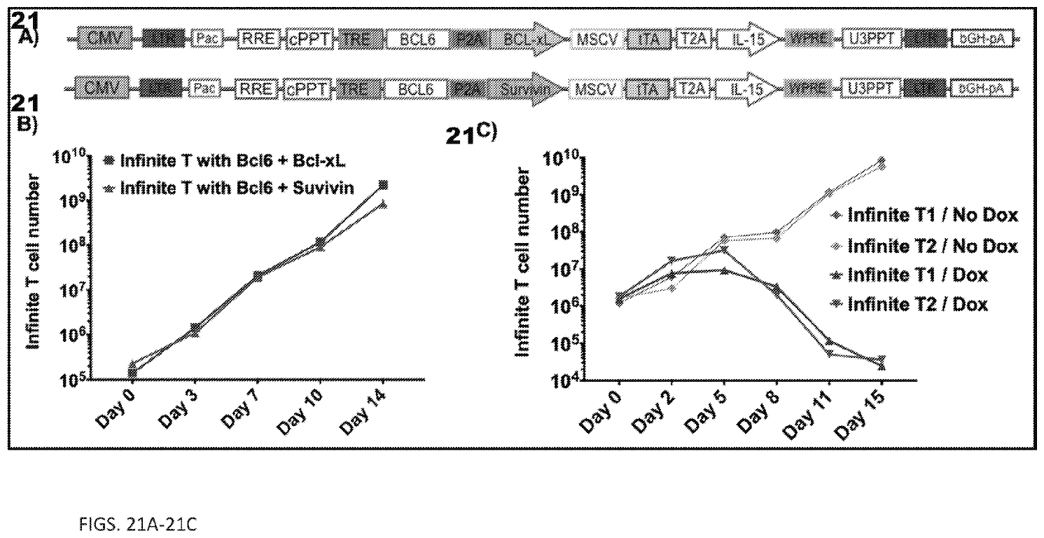

An example of a construct (L5x(MSCV-BCL6-P2A-BCL-xl-T2A-rtTA); see FIG. 21) that includes BCL6 with Bcl-xl is below. The general structure is as follows:

NNNN-CMV promoterNN-HIV-LTR-HIV1_psi pack-Spacer-RRE-spacer-cPPT-MSCV Promoter-BCL-6 WT-P2A-BCL-xL-T2A-rtTA-WPRE-U3PPT-HIV-LTR-bGH pA-SV40 origin of replication-Origin of plasmid replication-Ampicin resistance gene-AmpR_promoter-NNNN. Specific sequences of particular domains of the construct below (and in FIG. 21) are delineated immediately following SEQ ID NO:11 below:

(SEQ ID NO: 11)
GTCGACGGATCGGGAGATCTCCCGATCCCCTATGGTGCACTCTC
AGTACAATCTGCTCTGATGCCGCATAGTTAAGCCAGTATCTGCTCCCTGCTTGTGTGT
TGGAGGTCGCTGAGTAGTGCGCGAGCAAAATTTAAGCTACAACAAGGCAAGGCTTG
ACCGACAATTGCATGAAGAATCTGCTTAGGGTTAGGCGTTTTGCGCTGCTTCGCGAT
GTACGGGCCAGATATtCGCGTTGACATTGATTATTGACTAGTTATTAATAGTAATCAA
TTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGT
ATTTACGGTAAACTGCCCACTTGGCAGTACATCAAGTGTATCATATGCCAAGTACGC
CCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCAT
GGTGATGCGGTTTTGGCAGTACATCAATGGGCGTGGATAGCGGTTTGACTCACGGGG
ATTTCCAAGTCTCCACCCCATTGACGTCAATGGGAGTTTGTTTTGGCACCAAAATCA
ACGGGACTTTCCAAAATGTCGTAACAACTCCGCCCCATTGACGCAAATGGGCGGTA
GGCGTGTACGGTGGGAGGTCTATATAAGCAGCGCGTTTTGCCTGTACTGGGTCTCTC
TGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGT
GACTCTGGTAACTAGAGATCCCTCAGACCCTTTTAGTCAGTGTGGAAAATCTCTAGC
AGTGGCGCCCGAACAGGGACTTGAAAGCGAAAGGGAAACCAGAGGAGCTCTCTCG
ACGCAGGACTCGGCTTGCTGAAGCGCGCACGGCAAGAGGCGAGGGGCGGCGACTG
GTGAGTACGCCAAAAATTTTGACTAGCGGAGGCTAGAAGGAGAGAGATGGGTGCGA
GAGCGTCAGTATTAAGCGGGGGAGAATTAGATCGCGATGGGAAAAAATTCGGTTAA
GGCCAGGGGGAAAGAAAAAATATAAATTAAAACATATAGTATGGGCAAGCAGGGA
GCTAGAACGATTCGCAGTTAATCCTGGCCTGTTAGAAACATCAGAAGGCTGTAGAC
AAATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAGAC
ACCAAGGAAGCTTTAGACAAGATAGAGGAAGAGCAAAACAAAAGTAAGACCACCG
CACAGCAAGCGGCCGCTGATCTTCAGACCTGGAGGAGGAGATATGAGGGACAATTG
GAGAAGTGAATTATATAAATATAAAGTAGTAAAAATTGAACCATTAGGAGTAGCAC
CCACCAAGGCAAAGAGAAGAGTGGTGCAGAGAGAAAAAAGAGCAGTGGGAATAGG
AGCTTTGTTCCTTGGGTTCTTGGGAGCAGCAGGAAGCACTATGGGCGCAGCGTCAAT
GACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCA
TCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGG
AATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGA
ATCGCAAAACCAGCAAGAAAAGAATGAACAAGAATTATTGGAATTAGATAAATGGG
CAAGTTTGTGGAATTGGTTTAACATAACAAATTGGCTGTGGTATATAAAATTATTCA
TAATGATAGTAGGAGGCTTGGTAGGTTTAAGAATAGTTTTTGCTGTACTTTCTATAGT
GAATAGAGTTAGGCAGGGATATTCACCATTATCGTTTCAGACCCACCTCCCAACCCC
GAGGGGACCCGACAGGCCCGAAGGAATAGAAGAAGAAGGTGGAGAGAGAGACAG
AGACAGATCCATTCGATTAGTGAACGGATCGGCACTGCGTGCGCCAATTCTGCAGAC
AAATGGCAGTATTCATCCACAATTTTAAAAGAAAAGGGGGGATTGGGGGGTACAGT
GCAGGGGAAAGAATAGTAGACATAATAGCAACAGACATACAAACTAAAGAATTAC
AAAAACAAATTACAAAAATTCAAAATTTTCGGGTTTATTACAGGGACAGCAGAGAT
CCAGTTTGGTTAATtaatgaaagaccccacctgtaggtttggcaagctagcttaagtaacgccattttgcaaggcatggaaaata
cataactgagaatagagaagttcagatcaaggttaggaacagagagacagcagaatatgggccaaacaggatatctgtggtaagcagttcc
tgccccggctcagggccaagaacagatggtccccagatgcggtcccgccctcagcagtttctagagaaccatcagatgtttccagggtgcc
ccaaggacctgaaatgaccctgtgccttatttgaactaaccaatcagttcgcttctcgcttctgttcgcgcgcttctgctccccgagctcaataa
aagagcccacaacccctcactcggcgcgccagtcctccgatagactgcgtcgcccgggtacccgtattcccaataaagcctcttgctgtttg
catccgaatcgtggactcgctgatccttgggagggtctcctcagattgattgactgcccacctcgggggtctttcatcctaGGCTAGCc
accATGgcctcgccggctgacagctgtatccagttcacccgccatgccagtgatgttcttctcaaccttaatcgtctccggagtcgagacat
cttgactgatgttgtcattgttgtgagccgtgagcagtttagagcccataaaacggtcctcatggcctgcagtggcctgttctatagcatctttac
agaccagttgaaatgcaaccttagtgtgatcaatctagatcctgagatcaaccctgagggattctgcatcctcctggacttcatgtacacatctc
ggctcaatttgcgggagggcaacatcatggctgtgatggccacggctatgtacctgcagatggagcatgttgtggacacttgccggaagttt
attaaggccagtgaagcagagatggtttctgccatcaagcctcctcgtgaagagttcctcaacagccggatgctgatgccccaagacatcat
ggcctatcggggtcgtgaggtggtggagaacaacctgccactgaggagcgcccctgggtgtgagagcagagcctttgcccccagcctgt
acagtggcctgtccacaccgccagcctcttattccatgtacagccacctccctgtcagcagcctcctcttctccgatgaggagtttcgggatgt
ccggatgcctgtggccaaccccttccccaaggagcgggcactcccatgtgatagtgccaggccagtccctggtgagtacagccggccga
ctttggaggtgtcccccaatgtgtgccacagcaatatctattcacccaaggaaacaatcccagaagaggcacgaagtgatatgcactacagt
gtggctgagggcctcaaacctgctgccccctcagcccgaaatgccccctacttcccttgtgacaaggccagcaaagaagaagagagacc
ctcctcggaagatgagattgccctgcatttcgagccccccaatgcacccctgaaccggaagggtctggttagtccacagagcccccagaaa
tctgactgccagcccaactcgcccacagagtcctgcagcagtaagaatgcctgcatcctccaggcttctggctcccctccagccaagagcc
ccactgaccccaaagcctgcaactggaagaaatacaagttcatcgtgctcaacagcctcaaccagaatgccaaaccagaggggcctgag
caggctgagctgggccgcctttccccacgagcctacacggccccacctgcctgccagccacccatggagcctgagaaccttgacctcca
gtccccaaccaagctgagtgccagcggggaggactccaccatcccacaagccagccggctcaataacatcgttaacaggtccatgacgg
gctctccccgcagcagcagcgagagccactcaccactctacatgcaccccccgaagtgcacgtcctgcggctctcagtccccacagcatg
cagagatgtgcctccacaccgctggccccacgttccctgaggagatgggagagacccagtctgagtactcagattctagctgtgagaacg
gggccttcttctgcaatgagtgtgactgccgcttctctgaggaggcctcactcaagaggcacacgctgcagacccacagtgacaaacccta
caagtgtgaccgctgccaggcctccttccgctacaagggcaacctcgccagccacaagaccgtccataccggtgagaaaccctatcgttg
caacatctgtggggcccagttcaaccggccagccaacctgaaaacccacactcgaattcactctggagagaagccctacaaatgcgaaac
ctgcggagccagatttgtacaggtggcccacctccgtgcccatgtgcttatccacactggtgagaagccctatccctgtgaaatctgtggcac
ccgtttccggcaccttcagactctgaagagccacctgcgaatccacacaggagagaaaccttaccattgtgagaagtgtaacctgcatttcc
gtcacaaaagccagctgcgacttcacttgcgccagaagcatggcgccatcaccaacaccaaggtgcaataccgcgtgtcagccactgacc
tgcctccggagctccccaaagcctgcGGAAGCGGAGCTACTAACTTCAGCCTGCTGAAGCAGGCTG
GAGACGTGGAGGAGAACCCTGGACCTAGATCTGGAATGTCTCAGAGCAACCGGGAG
CTGGTGGTTGACTTTCTCTCCTACAAGCTTTCCCAGAAAGGATACAGCTGGAGTCAG
TTTAGTGATGTGGAAGAGAACAGGACTGAGGCCCCAGAAGGGACTGAATCGGAGAT
GGAGACCCCCAGTGCCATCAATGGCAACCCATCCTGGCACCTGGCAGACAGCCCCG
CGGTGAATGGAGCCACTGGCCACAGCAGCAGTTTGGATGCCCGGGAGGTGATCCCC
ATGGCAGCAGTAAAGCAAGCGCTGAGGGAGGCAGGCGACGAGTTTGAACTGCGGTA
CCGGCGGGCATTCAGTGACCTGACATCCCAGCTCCACATCACCCCAGGGACAGCAT
ATCAGAGCTTTGAACAGGTAGTGAATGAACTCTTCCGGGATGGGGTAAACTGGGGT
CGCATTGTGGCCTTTTTCTCCTTCGGCGGGGCACTGTGCGTGGAAAGCGTAGACAAG
GAGATGCAGGTATTGGTGAGTCGGATCGCAGCTTGGATGGCCACTTACCTGAATGAC
CACCTAGAGCCTTGGATCCAGGAGAACGGCGGCTGGGATACTTTTGTGGAACTCTAT
GGGAACAATGCAGCAGCCGAGAGCCGAAAGGGCCAGGAACGCTTCAACCGCTGGTT
CCTGACGGGCATGACTGTGGCCGGCGTGGTTCTGCTGGGCTCACTCTTCAGTCGGAA
AACGCGTGGCAGTggcgagggtagaggttctctcctcacttgtggtgatgttgaagaaaaccctggtccaatgtctagactgga
caagagcaaagtcataaacggagctctggaattactcaatggtgtcggtatcgaaggcctgacgacaaggaaactcgctcaaaagctggg
agttgagcagcctaccctgtactggcacgtgaagaacaagcgggccctgctcgatgccctgccaatcgagatgctggacaggcatcatac
ccacttctgccccctggaaggcgagtcatggcaagactttctgcggaacaacgccaagtcataccgctgtgctctcctctcacatcgcgacg
gggctaaagtgcatctcggcacccgcccaacagagaaacagtacgaaaccctggaaaatcagctcgcgttcctgtgtcagcaaggcttctc
cctggagaacgcactgtacgctctgtccgccgtgggccactttacactgggctgcgtattggaggaacaggagcatcaagtagcaaaaga
ggaaagagagacacctaccaccgattctatgcccccacttctgagacaagcaattgagctgttcgaccggcagggagccgaacctgccttc
cttttcggcctggaactaatcatatgtggcctggagaaacagctaaagtgcgaaagcggcgggccgaccgacgcccttgacgattttgactt
agacatgctcccagccgatgcccttgacgactttgaccttgatatgctgcctgctgacgctcttgacgattttgaccttgacatgctccccgggt
aaGGTgACCGATATCAAGCTTATCGATAATCAACCTCTGGATTACAAAATTTGTGAAA
GATTGACTGGTATTCTTAACTATGTTGCTCCTTTTACGCTATGTGGATACGCTGCTTT
AATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCACCACCT
GTCAGCTCCTTTCCGGGACTTTCGCTTTCCCCCTCCCTATTGCCACGGCGGAACTCAT
CGCCGCCTGCCTTGCCCGCTGCTGGACAGGGGCTCGGCTGTTGGGCACTGACAATTC
CGTGGTGTTGTCGGGGAAATCATCGTCCTTTCCTTGGCTGCTCGCCTGTGTTGCCACC
TGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCC
CTCAGACGAGTCGGATCTCCCTTTGGGCCGCCTCCCCGCACTCGAGACCTAGAAAAA
CATGGAGCAATCACAAGTAGCAATACAGCAGCTACCAATGCTGATTGTGCCTGGCT
AGAAGCACAAGAGGAGGAGGAGGTGGGTTTTCCAGTCACACCTCAGGTACCTTTAA
GACCAATGACTTACAAGGCAGCTGTAGATCTTAGCCACTTTTTAAAAGAAAAGGGG
GGACTGGAAGGGCTAATTCACTCCCAACGAAGACAAGATATCCTTGATCTGTGGATC
TACCACACACAAGGCTACTTCCCTGATTGGCAGAACTACACACCAGGGCCAGGGAT
CAGATATCCACTGACCTTTGGATGGTGCTACAAGCTAGTACCAGTTGAGCAAGAGA
AGGTAGAAGAAGCCAATGAAGGAGAGAACACCCGCTTGTTACACCCTGTGAGCCTG
CATGGGATGGATGACCCGGAGAGAGAAGTATTAGAGTGGAGGTTTGACAGCCGCCT
AGCATTTCATCACATGGCCCGAGAGCTGCATCCGGACTGTACTGGGTCTCTCTGGTT
AGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTC
TGGTAACTAGAGATCCCTCAGACCCTTTTAGTCAGTGTGGAAAATCTCTAGCAGGGC
CCGTTTAAACCCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATG
CTGGGGATGCGGTGGGCTCTATGGCATGTCTatcccgcccctaactccgcccagttccgcccattctccgccc
catggctgactaattttttttatttatgcagaggccgaggccgcctcggcctctgagctattccagaagtagtgaggaggcttttttggaggcc
GTATACCGTCGACCTCTAGCTAGAGCTTGGCGTAATCATGGTCATAGCTGTTTCCTGT
GTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCAGTGGAACGA
AAACTCACGTTAAGGGATTTTGGTCATGAGATTATCAAAAAGGATCTTCACCTAGAT
CCTTTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAG
TTCGCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATA
CGGGATAATACCGCGCCACATAGCAGAACTTTAAAAGTGCTCATCATTGGAAAACG
TTCTTCGGGGCGAAAACTCTCAAGGATCTTACCGCTGTTGAGATCCAGTTCGATGTA
ACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGT
TCCGCGCACATTTCCCCGAAAAGTGCCACCTGAC
CMV promoter
(SEQ ID NO: 61)
ACATTGATTATTGACTAGTTATTAATAGTAATCAATTACGGGGT
CATTAGTTCATAGCCCATATATGGAGTTCCGCGTTACATAACTTACGGTAAATGGCC
CGCCTGGCTGACCGCCCAACGACCCCCGCCCATTGACGTCAATAATGACGTATGTTC
CCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTT
TCCAAAATGTCGTAACAACTCCGCCCCATTGACGCAAATGGGCGGTAGGCGTGTAC
GGTGGGAGGTCTATATAAGC
HIV LTR
(SEQ ID NO: 62)
GGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGT
AGTGTGTGCCCGTCTGTTGTGTGACTCTGGTAACTAGAGATCCCTCAGACCCTTTTAG
TCAGTGTGGAAAATCTCTAGCA
HIV1 psi pack
(SEQ ID NO: 63)
TGAGTACGCCAAAAATTTTGACTAGCGGAGGCTAGAAGGAGAGAG
RRE
(SEQ ID NO: 64)
AGGAGCTTTGTTCCTTGGGTTCTTGGGAGCAGCAGGAAGCACTA
TGGGCGCAGCGTCAATGACGCTGACGGTACAGGCCAGACAATTATTGTCTGGTATA
GTGCAGCAGCAGAACAATTTGCTGAGGGCTATTGAGGCGCAACAGCATCTGTTGCA
ACTCACAGTCTGGGGCATCAAGCAGCTCCAGGCAAGAATCCTGGCTGTGGAAAGAT
ACCTAAAGGATCAACAGCTCCT
cPPT
(SEQ ID NO: 65)
AAAAGAAAAGGGGGGA
MSCV Promoter
(SEQ ID NO: 66)
aatgaaagaccccacctgtaggtttggcaagctagcttaagtaacgccattttgcaaggcatggaaaatac
ataactgagaatagagaagttcagatcaaggttaggaacagagagacagcagaatatgggccaaacaggatatctgtggtaagcagttcct
gccccggctcagggccaagaacagatggtccccagatgcggtcccgccctcagcagtttctagagaaccatcagatgtttccagggtgcc
ccaaggacctgaaatgaccctgtgccttatttgaactaaccaatcagttcgcttctcgcttctgttcgcgcgcttctgctccccgagctcaataa
aagagcccacaacccctcactcggcgcgccagtcctccgatagactgcgtcgcccgggtacccgtattcccaataaagcctcttgctgtttg
catccgaatcgtggactcgctgatccttgggagggtctcctcagattgattgactgcccacctcgggggtctttcat
BCL-6 WT
(SEQ ID NO: 67)
ATGgcctcgccggctgacagctgtatccagttcacccgccatgccagtgatgttcttctcaaccttaatc
gtctccggagtcgagacatcttgactgatgttgtcattgttgtgagccgtgagcagtttagagcccataaaacggtcctcatggcctgcagtgg
cctgttctatagcatctttacagaccagttgaaatgcaaccttagtgtgatcaatctagatcctgagatcaaccctgagggattctgcatcctcct
ggacttcatgtacacatctcggctcaatttgcgggagggcaacatcatggctgtgatggccacggctatgtacctgcagatggagcatgttgt
ggacacttgccggaagtttattaaggccagtgaagcagagatggtttctgccatcaagcctcctcgtgaagagttcctcaacagccggatgc
tgatgccccaagacatcatggcctatcggggtcgtgaggtggtggagaacaacctgccactgaggagcgcccctgggtgtgagagcaga
gcctttgcccccagcctgtacagtggcctgtccacaccgccagcctcttattccatgtacagccacctccctgtcagcagcctcctcttctccg
atgaggagtttcgggatgtccggatgcctgtggccaaccccttccccaaggagcgggcactcccatgtgatagtgccaggccagtccctg
gtgagtacagccggccgactttggaggtgtcccccaatgtgtgccacagcaatatctattcacccaaggaaacaatcccagaagaggcac
gaagtgatatgcactacagtgtggctgagggcctcaaacctgctgccccctcagcccgaaatgccccctacttcccttgtgacaaggccag
caaagaagaagagagaccctcctcggaagatgagattgccctgcatttcgagccccccaatgcacccctgaaccggaagggtctggttag
tccacagagcccccagaaatctgactgccagcccaactcgcccacagagtcctgcagcagtaagaatgcctgcatcctccaggcttctggc
tcccctccagccaagagccccactgaccccaaagcctgcaactggaagaaatacaagttcatcgtgctcaacagcctcaaccagaatgcc
aaaccagaggggcctgagcaggctgagctgggccgcctttccccacgagcctacacggccccacctgcctgccagccacccatggagc
ctgagaaccttgacctccagtccccaaccaagctgagtgccagcggggaggactccaccatcccacaagccagccggctcaataacatc
gttaacaggtccatgacgggctctccccgcagcagcagcgagagccactcaccactctacatgcaccccccgaagtgcacgtcctgcgg
ctctcagtccccacagcatgcagagatgtgcctccacaccgctggccccacgttccctgaggagatgggagagacccagtctgagtactc
agattctagctgtgagaacggggccttcttctgcaatgagtgtgactgccgcttctctgaggaggcctcactcaagaggcacacgctgcaga
cccacagtgacaaaccctacaagtgtgaccgctgccaggcctccttccgctacaagggcaacctcgccagccacaagaccgtccataccg
gtgagaaaccctatcgttgcaacatctgtggggcccagttcaaccggccagccaacctgaaaacccacactcgaattcactctggagagaa
gccctacaaatgcgaaacctgcggagccagatttgtacaggtggcccacctccgtgcccatgtgcttatccacactggtgagaagccctatc
cctgtgaaatctgtggcacccgtttccggcaccttcagactctgaagagccacctgcgaatccacacaggagagaaaccttaccattgtgag
aagtgtaacctgcatttccgtcacaaaagccagctgcgacttcacttgcgccagaagcatggcgccatcaccaacaccaaggtgcaatacc
gcgtgtcagccactgacctgcctccggagctccccaaagcctgc
P2A
(SEQ ID NO: 68)
GGAAGCGGAGCTACTAACTTCAGCCTGCTGAAGCAGGCTGGAG
ACGTGGAGGAGAACCCTGGACCT
BCL-xL
(SEQ ID NO: 69)
AGATCTGGAATGTCTCAGAGCAACCGGGAGCTGGTGGTTGACT
TTCTCTCCTACAAGCTTTCCCAGAAAGGATACAGCTGGAGTCAGTTTAGTGATGTGG
AAGAGAACAGGACTGAGGCCCCAGAAGGGACTGAATCGGAGATGGAGACCCCCAG
TGCCATCAATGGCAACCCATCCTGGCACCTGGCAGACAGCCCCGCGGTGAATGGAG
CCACTGGCCACAGCAGCAGTTTGGATGCCCGGGAGGTGATCCCCATGGCAGCAGTA
AAGCAAGCGCTGAGGGAGGCAGGCGACGAGTTTGAACTGCGGTACCGGCGGGCATT
CAGTGACCTGACATCCCAGCTCCACATCACCCCAGGGACAGCATATCAGAGCTTTGA
ACAGGTAGTGAATGAACTCTTCCGGGATGGGGTAAACTGGGGTCGCATTGTGGCCTT
TTTCTCCTTCGGCGGGGCACTGTGCGTGGAAAGCGTAGACAAGGAGATGCAGGTATT
GGTGAGTCGGATCGCAGCTTGGATGGCCACTTACCTGAATGACCACCTAGAGCCTTG
GATCCAGGAGAACGGCGGCTGGGATACTTTTGTGGAACTCTATGGGAACAATGCAG
CAGCCGAGAGCCGAAAGGGCCAGGAACGCTTCAACCGCTGGTTCCTGACGGGCATG
ACTGTGGCCGGCGTGGTTCTGCTGGGCTCACTCTTCAGTCGGAAA
T2A
(SEQ ID NO: 70)
GGCAGTggcgagggtagaggttctctcctcacttgtggtgatgttgaagaaaaccctggtcca
rtTA
(SEQ ID NO: 71)
atgtctagactggacaagagcaaagtcataaacggagctctggaattactcaatggtgtcggtatcgaagg
cctgacgacaaggaaactcgctcaaaagctgggagttgagcagcctaccctgtactggcacgtgaagaacaagcgggccctgctcgatg
ccctgccaatcgagatgctggacaggcatcatacccacttctgccccctggaaggcgagtcatggcaagactttctgcggaacaacgccaa
gtcataccgctgtgctctcctctcacatcgcgacggggctaaagtgcatctcggcacccgcccaacagagaaacagtacgaaaccctgga
aaatcagctcgcgttcctgtgtcagcaaggcttctccctggagaacgcactgtacgctctgtccgccgtgggccactttacactgggctgcgt
attggaggaacaggagcatcaagtagcaaaagaggaaagagagacacctaccaccgattctatgcccccacttctgagacaagcaattga
gctgttcgaccggcagggagccgaacctgccttccttttcggcctggaactaatcatatgtggcctggagaaacagctaaagtgcgaaagc
ggcgggccgaccgacgcccttgacgattttgacttagacatgctcccagccgatgcccttgacgactttgaccttgatatgctgcctgctgac
gctcttgacgattttgaccttgacatgctccccgggtaaGGTgA
WPRE
(SEQ ID NO: 72)
TCAACCTCTGGATTACAAAATTTGTGAAAGATTGACTGGTATTC
TTAACTATGTTGCTCCTTTTACGCTATGTGGATACGCTGCTTTAATGCCTTTGTATCA
TGCTATTGCTTCCCGTATGGCTTTCATTTTCTCCTCCTTGTATAAATCCTGGTTGCTGT
CTCTTTATGAGGAGTTGTGGCCCGTTGTCAGGCAACGTGGCGTGGTGTGCACTGTGT
TTGCTGACGCAACCCCCACTGGTTGGGGCATTGCCACCACCTGTCAGCTCCTTTCCG
GGACTTTCGCTTTCCCCCTCCCTATTGCCACGGCGGAACTCATCGCCGCCTGCCTTGC
CCGCTGCTGGACAGGGGCTCGGCTGTTGGGCACTGACAATTCCGTGGTGTTGTCGGG
GAAATCATCGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTCCCGCGGCC
TGCTGCCGGCTCTGCGGCCTCTTCCGCGTCTTCGCCTTCGCCCTCAGACGAGTCGGAT
CTCCCTTTGGGCCGCCTCCCCGCA
U3PPT
(SEQ ID NO: 73)
AAAAGAAAAGGGGGGA
- HIV-LTR
(SEQ ID NO: 74)
GGGTCTCTCTGGTTAGACCAGATCTGAGCCTGGGAGCTCTCTGG
CTAACTAGGGAACCCACTGCTTAAGCCTCAATAAAGCTTGCCTTGAGTGCTTCAAGT
AGTGTGTGCCCGTCTGTTGTGTGACTCTGGTAACTAGAGATCCCTCAGACCCTTTTAG
TCAGTGTGGAAAATCTCTAGCA
bGH pA
(SEQ ID NO: 75)
CGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATG
SV40 origin of replication
(SEQ ID NO: 76)
Atcccgcccctaactccgcccagttccgcccattctccgccccatggctgactaattttttttatttatgcaga
ggccgaggccgcctcggcctctgagctattccagaagtagtgaggaggcttttttggaggcc
Origin of plasmid replication
(SEQ ID NO: 77)
TTTCCATAGGCTCCGCCCCCCTGACGAGCATCACAAAAATCGAC
GCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAG
CAGCAGATTACGCGCAGAAAAAAAGGATCTCAA
Ampicillin resistance gene
(SEQ ID NO: 78)
TTACCAATGCTTAATCAGTGAGGCACCTATCTCAGCGATCTGTC
TATTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGG
TCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTG
CATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGAC
ACGGAAATGTTGAATACTCAT
AmpR_promoter
(SEQ ID NO: 79)
ATTGTCTCATGAGCGGATACATATTTGAA

In further aspects, the present disclosure provides infinite immune cells that can be genetically modified to confer a disposition to favor the targeting of the infinite immune cells to specific organ sites or tumor markers. The infinite immune cells may express one or more suicide or elimination genes that could be used to eliminate infinite immune cells from patients in case of serious adverse events. The infinite immune cells may express one or more genes including genes encoding IL-2 and/or IL-15 that could maintain or enhance the proliferation of infinite T cells for in vivo applications. The expression of IL-2 and/or IL-15 might be constitutive expression or otherwise regulatable, such as doxycycline regulatable (Tet-on or Tet-off). The cells might be engineered to express other one or more other cytokines such as IL-7, IL-12, IL-18, IL-21, etc; one or more chemokine receptors such as CCR1, CCR4, CCR5, CCR6, CCR7, CCR9, CCR10, CXCR1, CXCR2, CXCR3, CXCR4, CXCR5, CXCR7 (ACKR3), CX3CR1, CCRL2 (ACKR5), etc. and/or one or more other chemokines such as CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, CXCL4L1, etc., for example.

Infinite immune cells may be modified to express antigen-specific CARs or TCRs to target tumors or infections. Another strategy to target tumors may be to modify infinite T cells to express a CAR with an Fc receptor on the extracellular domain so that they can then be used in conjunction with monoclonal antibodies against a tumor marker. In addition, infinite immune cells may be modified to express specific chemokine receptors and/or adhesion molecules including integrins, selectins, adhesion molecules belonging to the immunoglobulin superfamily, cadherins, and the CD44 family to preferentially direct the trafficking of these cells to organ sites of interest.

A further embodiment provides infinite immune cells with one or more safety switches, such as a suicide gene or elimination gene of any kind. In some embodiments, the system may utilize truncated human epidermal growth factor receptor (hEGFRt), HSV-TK, SR39 mutant HSV-TK, the yeast CD gene or its mutant CD20. In cases where hEGFRt is utilized, this gene can give infinite T cells the characteristic to be recognized and eliminated by an FDA-approved monoclonal antibody, such as cetuximab, when they are not needed. For example, this gene can serve as a safety switch when serious adverse events occur after injection of therapeutic infinite immune cells. In addition to serving as a safety switch, the hEGFRt can also serve as a marker to enrich CAR positive cells and to track these cells following infusion into patients.

One example of a truncated EGFR is below in which case domains 1 and 2 of EGFR have been deleted:

DNA sequence:

(SEQ ID NO: 12)
5-ATGCTGCTGCTGGTGACCAGCCTGCTGCTGTGCGAGCTGCCACACCCT
GCCTTCCTGAGGAAAGTGTGTAATGGCATCGGCATCGGCGAGTTTAAGGA
CAGCCTGTCCATCAACGCCACAAATATCAAGCACTTCAAGAACTGTACCT
CTATCAGCGGCGACCTGCACATCCTGCCAGTGGCCTTCAGAGGCGATTCC
TTTACACACACCCCACCACTGGACCCACAGGAGCTGGATATCCTGAAGAC
AGTGAAGGAGATCACCGGCTTCCTGCTGATCCAGGCATGGCCAGAGAACA
GGACAGATCTGCACGCCTTTGAGAATCTGGAGATCATCAGAGGCAGGACC
AAGCAGCACGGCCAGTTCTCTCTGGCCGTGGTGAGCCTGAACATCACATC
CCTGGGCCTGCGCTCTCTGAAGGAGATCAGCGACGGCGATGTGATCATCT
CCGGCAACAAGAATCTGTGCTATGCCAACACCATCAATTGGAAGAAGCTG
TTTGGCACATCTGGCCAGAAGACCAAGATCATCAGCAACCGCGGCGAGAA
TTCCTGCAAGGCAACCGGACAGGTGTGCCACGCACTGTGTAGCCCTGAGG
GATGTTGGGGACCAGAGCCACGCGACTGCGTGTCCTGTAGGAACGTGTCT
AGGGGAAGGGAGTGCGTGGATAAGTGTAATCTGCTGGAGGGAGAGCCAAG
GGAGTTCGTGGAGAACTCCGAGTGCATCCAGTGTCACCCCGAGTGCCTGC
CTCAGGCCATGAACATCACATGTACCGGCCGGGGCCCTGACAATTGCATC
CAGTGTGCCCACTACATCGATGGCCCTCACTGCGTGAAGACATGTCCAGC
CGGCGTGATGGGCGAGAACAATACCCTGGTGTGGAAGTATGCAGACGCAG
GACACGTGTGCCACCTGTGTCACCCCAATTGCACATACGGATGTACCGGA
CCAGGACTGGAGGGATGTCCTACAAACGGCCCTAAGATCCCAAGCATCGC
AACCGGAATGGTGGGAGCACTGCTGCTGCTGCTGGTGGTGGCACTGGGAA
TCGGACTGTTCATGAGGCGGTGA-3

Amino acid sequence of a truncated EGFR lacking domains 1 and 2:

(SEQ ID NO: 13)
MLLLVTSLLLCELPHPAFLRKVCNGIGIGEFKDSLSINATNIKHFKNCTS
ISGDLHILPVAFRGDSFTHTPPLDPQELDILKTVKEITGFLLIQAWPENR
TDLHAFENLEIIRGRTKQHGQFSLAVVSLNITSLGLRSLKEISDGDVIIS
GNKNLCYANTINWKKLFGTSGQKTKIISNRGENSCKATGQVCHALCSPEG
CWGPEPRDCVSCRNVSRGRECVDKCNLLEGEPREFVENSECIQCHPECLP
QAMNITCTGRGPDNCIQCAHYIDGPHCVKTCPAGVMGENNTLVWKYADAG
HVCHLCHPNCTYGCTGPGLEGCPTNGPKIPSIATGMVGALLLLLVVALGI
GLFMRR

In certain embodiments, a fusion protein as a safety switch is a fusion of EGFR (domain 3) and HER2 (domain IV) fusion protein. In such cases, the EGFR domain 3 is the antibody binding domain and the HER2 domain 4 contains the extracellular spacer and transmembrane domain. In specific embodiments, this fusion protein is a separate molecule from the CAR.

Any one or more genes or expression constructs in the infinite cells may or may not be regulatable, such as by a Tet-on or Tet-off system in a doxycycline regulatable manner. An example of a sequence of a Tet-responsive promoter includes the following Tet responsive promoter that contains 7 repeats of Tet responsive elements:

(SEQ ID NO: 14)
gagtttactccctatcagtgatagagaacgtatgtcgagtttactccct
atcagtgatagagaacgatgtcgagtttactccctatcagtgatagaga
acgtatgtcgagtttactccctatcagtgatagagaacgtatgtcgagt
ttactccctatcagtgatagagaacgtatgtcgagtttatccctatcag
tgatagagaacgtatgtcgagtttactccctatcagtgatagagaacgt
atgtcgaggtaggcgtgtacggtgggaggcctatataagcagagctcgt
ttagtgaaccgtcagatcgcc

For the tet system, an example of DNA sequence for tTA(Tet off) is as follows:

(SEQ ID NO: 15)
ATGAGCCGCCTGGATAAGTCCAAAGTGATCAACTCTGCCCTGGAGCTGCT
GAATGAAGTGGGCATCGAGGGCCTGACCACACGGAAGCTGGCCCAGAAGC
TGGGAGTGGAGCAGCCAACCCTGTACTGGCACGTGAAGAACAAGCGCGCC
CTGCTGGACGCCCTGGCCATCGAGATGCTGGATCGGCACCACACACACTT
CTGCCCCCTGGAGGGAGAGTCCTGGCAGGATTTCCTGCGGAACAATGCCA
AGAGCTTTAGATGTGCACTGCTGTCCCACAGGGACGGAGCAAAGGTGCAC
CTGGGCACCAGGCCTACAGAGAAGCAGTACGAGACCCTGGAGAACCAGCT
GGCCTTCCTGTGCCAGCAGGGCTTTTCTCTGGAGAATGCACTGTATGCAC
TGAGCGCCGTGGGACACTTCACCCTGGGATGCGTGCTGGAGGACCAGGAG
CACCAGGTGGCCAAGGAGGAGAGAGAGACACCCACCACAGATTCCATGCC
CCCTCTGCTGAGGCAGGCCATCGAGCTGTTTGACCACCAGGGAGCAGAGC
CTGCCTTCCTGTTTGGCCTGGAGCTGATCATCTGCGGCCTGGAGAAGCAG
CTGAAGTGTGAGTCTGGAGGACCAGCAGACGCCCTGGACGATTTCGACCT
GGATATGCTGCCCGCCGATGCCCTGGACGATTTTGACCTGGATATGCTGC
CTGCCGACGCCCTGGACGATCTGGACCTGGATATGCTGCCAGGCacc

An example of amino acid sequence of tTA(Tet off) is as follows:

(SEQ ID NO: 16)
MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQE
HQVAKEERETPTTDSMPPLLRQAIELFDHQGAEPAFLFGLELIICGLEKQ
LKCESGGPADALDDFDLDMLPADALDDFDLDMLPADALDDLDLDMLPG

An example of DNA sequence of rtTA(Tet on) is as follows:

(SEQ ID NO: 17)
atgtctagactggacaagagcaaagtcataaacggagctctggaattact
caatggtgtcggtatcgaaggcctgacgacaaggaaactcgctcaaaagc
tgggagttgagcagcctaccctgtactggcacgtgaagaacaagcgggcc
ctgctcgatgccctgccaatcgagatgctggacaggcatcatacccactt
ctgccccctggaaggcgagtcatggcaagactttctgcggaacaacgcca
agtcataccgctgtgctctcctctcacatcgcgacggggctaaagtgcat
ctcggcacccgcccaacagagaaacagtacgaaaccctggaaaatcagct
cgcgttcctgtgtcagcaaggcttctccctggagaacgcactgtacgctc
tgtccgccgtgggccactttacactgggctgcgtattggaggaacaggag
catcaagtagcaaaagaggaaagagagacacctaccaccgattctatgcc
cccacttctgagacaagcaattgagctgttcgaccggcagggagccgaac
ctgccttccttttcggcctggaactaatcatatgtggcctggagaaacag
ctaaagtgcgaaagcggcgggccgaccgacgcccttgacgattttgactt
agacatgctcccagccgatgcccttgacgactttgaccttgatatgctgc
ctgctgacgctcttgacgattttgaccttgacatgctccccgggtaa

An example of amino acid sequence of rtTA(Tet on) is as follows:

(SEQ ID NO: 18)
MSRLDKSKVINGALELLNGVGIEGLTTRKLAQKLGVEQPTLYWHVKNKR
ALLDALPIEMLDRHHTHFCPLEGESWQDFLRNNAKSYRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGGPTDALDDFDLDMLPADALDDFDLDMLPADALDDFDLDM
LPG

In some aspects, the infinite immune cells may be be engineered to express one or more cytokiens, including IL-2 and/or IL-15, such as inducible IL-2 and/or IL-15, such as to maintain or enhance proliferation. In specific cases, however, any cytokine in the system may be regulated constitutively. For example, infinite immune cells could produce IL-15 and/or IL-2 in the presence of the induction agent, such as doxycycline, to support their own proliferation. By adjusting the dosage of doxycycline, the survival and proliferation of infinite immune cells can be maintained or regulated in vivo.

Particular IL-2 sequences may be utilized. In at least some cases, IL-2 has two examples of DNA sequences, and both of them encode the same IL-2 amino acid sequence.

IL-2 DNA Sequence 1:

(SEQ ID NO: 19)
ATGTATCGGATGCAACTCCTCAGCTGCATTGCGTTGTCACTCGCACTCGT
CACGAACTCTGCACCGACATCTAGTAGTACTAAGAAAACACAGTTGCAAC
TGGAGCACCTGCTGTTGGATTTGCAAATGATCCTTAACGGGATCAACAAC
TACAAAAACCCTAAGCTCACACGAATGCTTACTTTCAAGTTTTACATGCC
GAAAAAAGCCACAGAGCTGAAGCATCTTCAGTGCCTTGAAGAGGAGCTTA
AACCCCTCGAGGAGGTACTGAATCTCGCGCAAAGCAAGAATTTTCATTTG
CGGCCCCGGGACCTTATATCAAACATTAACGTGATCGTGTTGGAACTCAA
GGGATCAGAGACGACATTTATGTGCGAGTACGCTGACGAGACCGCTACAA
TCGTAGAGTTTCTCAATAGGTGGATCACGTTTTGCCAAAGCATCATCTCA
ACGCTC

IL-2 DNA Sequence 2:

(SEQ ID NO: 20)
ATGTATAGGATGCAGCTGCTGTCCTGCATCGCCTTGTCCCTGGCCCTTGT
GACCAACAGCGCCCCAACCTCCTCCTCTACCAAAAAAACCCAACTTCAGC
TTGAGCATCTCCTCTTGGACCTGCAGATGATCCTGAATGGTATAAACAAC
TACAAGAACCCCAAGCTGACCCGGATGCTTACATTCAAATTCTATATGCC
TAAAAAGGCTACAGAGCTGAAGCACCTGCAGTGCCTGGAAGAGGAGCTGA
AGCCACTGGAAGAGGTCCTGAACTTGGCCCAGAGCAAGAACTTTCACCTC
AGGCCCAGGGACTTGATAAGCAACATAAATGTAATCGTCCTGGAGCTGAA
GGGGTCTGAAACAACCTTCATGTGTGAGTATGCAGATGAGACCGCTACCA
TCGTGGAGTTCCTCAACAGATGGATTACATTTTGTCAATCCATCATCAGC
ACCCTGACATCT

In certain embodiments, a specific IL-2 amino acid sequence is utilized in the cells:

(SEQ ID NO: 21)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TL

In certain embodiments, a specific IL-15 nucleic acid polymer sequence is utilized in the cells:

(SEQ ID NO: 22)
ATGGGCCTGACCTCTCAGCTGCTGCCACCCCTGTTCTTTCTGCTGGCCT
GTGCCGGCAATTTCGTGCACGGCGCCAACTGGGTGAATGTGATCTCTGA
CCTGAAGAAGATCGAGGATCTGATCCAGAGCATGCACATCGACGCCACC
CTGTATACAGAGTCCGATGTGCACCCTTCTTGCAAGGTGACAGCCATGA
AGTGTTTTCTGCTGGAGCTGCAGGTCATCTCTCTGGAGAGCGGCGACGC
CAGCATCCACGATACCGTGGAGAATCTGATCATCCTGGCCAACAATAGC
CTGAGCTCCAACGGCAATGTGACAGAGTCCGGCTGCAAGGAGTGTGAGG
AGCTGGAGGAGAAGAACATCAAGGAGTTCCTGCAGTCCTTTGTGCACAT
CGTGCAGATGTTTATCAATACCTCTTGA

In certain embodiments, a specific IL-15 amino acid sequence is utilized in the cells:

(SEQ ID NO: 23)
MGLTSQLLPPLFFLLACAGNFVHGANWVNVISDLKKIEDLIQSMHIDATL
YTESDVHPSCKVTAMKCFLLELQVISLESGDASIHDTVENLIILANNSLS
SNGNVTESGCKECEELEEKNIKEFLQSFVHIVQMFINTS

In particular cases, the immune cells comprise IL-15 fused with part or all of the IL-15 receptor. In a specific case, the immune cells comprise IL-15 fused with the sushi domain of IL-15 receptor alpha unit, and an example of the sequence of which is as follows:

(SEQ ID NO: 24)
MAPRRARGCRTLGLPALLLLLLLRPPATRGITCPPPMSVEHADIWVKSYS
LYSRERYICNSGFKRKAGTSSLTECVLNKATNVAHWTTPSLKCIRDGGGG
SGGGGSGGGGSNWVNVISDLKKIEDLIQSMHIDATLYTESDVHPSCKVTA
MKCFLLELQVISLESGDASIHDTVENLIILANNSLSSNGNVTESGCKECE
ELEEKNIKEFLQSFVHIVQMFINTS

The DNA sequence of IL-15 fused with the sushi domain of IL-15 receptor alpha unit:

(SEQ ID NO: 25)
ATGGCACCTAGAAGAGCCAGAGGATGTAGAACACTGGGACTGCCAGCGCT
CCTTCTTTTGTTGCTGCTGAGACCACCTGCAACTCGCGGAATCACTTGTC
CTCCTCCTATGAGTGTGGAACACGCTGACATTTGGGTCAAGTCCTACTCT
CTGTATTCCCGGGAGAGATATATATGTAACTCTGGTTTCAAACGCAAGGC
AGGCACCAGCAGCCTTACCGAGTGTGTGCTTAACAAGGCAACAAATGTGG
CTCACTGGACAACACCTTCTCTGAAGTGCATTAGAGATGGAGGCGGAGGA
TCAGGTGGAGGAGGTTCTGGTGGGGGTGGATCAAATTGGGTGAACGTAAT
TTCCGACCTGAAAAAGATCGAAGATCTCATTCAAAGCATGCATATCGATG
CCACCCTCTATACCGAGAGCGATGTCCACCCATCCTGCAAAGTTACGGCG
ATGAAATGCTTCCTGCTCGAGCTCCAGGTTATTTCTCTGGAGAGCGGGGA
TGCCTCCATCCACGATACTGTCGAGAACCTCATTATTCTGGCCAATAACT
CCCTGTCTAGCAATGGCAATGTGACTGAATCAGGTTGCAAGGAGTGCGAG
GAGCTCGAAGAGAAAAACATAAAAGAATTCCTGCAATCCTTTGTCCATAT
CGTACAGATGTTTATCAACACCAGC

The infinite immune cells can be genetically engineered to give infinite cells target selectivity by introducing one or more chimeric antigen receptors (CARs) that can recognize a specific tumor marker such as CD19, CD20, CD22, and/or mesothelin; and/or T cell receptors (TCRs), such as TCRs against EBV, CMV, or NY-ESO-1. One example is ‘anti-CD19 infinite CART cells’ (CD19 inCART), referred to elsewhere herein. CD19 is expressed in almost all kinds of B cell lymphomas or B cell leukemias and normal B cells. CD19 in CART is produced by delivering lentiviral or non-viral vectors expressing anti-CD19 CAR into selected infinite cells.

The infinite immune cells can also be genetically engineered to confer additional properties such as i) resistance to T cell exhaustion by knocking out or knocking down inhibitory receptors or ligands PD-1, LAG-3, TIM-3, PD-L1, etc., ii) resistance to immunosuppressive mechanisms such as by knocking out or knocking down TGF-β receptor, iii) prevention of graft-versus-host disease by knocking out TCR, iv) improved efficacy by expressing surface or intracellular molecules such as cytokines or cytotoxic molecules, and v) improved persistence in vivo by making them resistant to elimination by host immune cells including T cells and NK cells. This may be achieved by knocking out or knocking down MHC molecules or by expressing surface ligands or other surface or intracellular molecules in infinite immune cells in order to suppress or diminish the function of host immune cells.

The infinite immune cells may be produced by a particular method or under particular conditions. For example, in specific embodiments, during the production of the infinite immune cells the cells while being produced may be subject to one or more particular agents that enhances their efficacy upon production, at least compared to their efficacy in the absence of exposure to the one or more particular agents. For example, in some cases, IL-2 is used to generate and expand infinite T cells. In specific embodiments, one or more different combinations of cytokines (IL-2, IL-7, IL-21, IL-15, IL-12, IL-18, IL-23, IFN-gamma, TNF-alpha, etc.) and/or chemokines may be utilized to prepare infinite T cells with particular phenotypes and particular functions.

### IV. GENETICALLY ENGINEERED ANTIGEN RECEPTORS

The immune cells of the present disclosure may or may not be genetically engineered to express one or more antigen receptors, such as one or more engineered TCRs and/or one or more CARs. For example, the immune cells may be modified to express a CAR and/or TCR having antigenic specificity for a cancer antigen or a microbial antigen, including a pathogenic antigen. Multiple CARs and/or TCRs, such as to different antigens, may be added to the immune cells. In some aspects, the immune cells are engineered to express the CAR or TCR by knock-in of the CAR or TCR at an inhibitory gene locus using gene editing methods such as CRISPR/Cas9.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

Electroporation of RNA coding for the full length TCR α and β (or γ and δ) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR α and β chain are only transiently expressed. When the introduced TCR α and β chain expression is diminished, only normal autologous T cells are left. This is not the case when full length TCR chains are introduced by stable retroviral transduction, which will never lose the introduced TCR chains, causing a constantly present autoreactivity in the patient.

In some embodiments, the cells comprise one or more nucleic acid polymers introduced via genetic engineering that encode one or more antigen receptors, and genetically engineered products of such nucleic acid polymers. In some embodiments, the nucleic acid polymers are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acid polymers are not naturally occurring, such as a nucleic acid polymer not found in nature (e.g., chimeric).

In some embodiments, the CAR comprises an extracellular antigen-recognition domain that specifically binds to one or more antigens. In some embodiments, the antigen is a protein, lipid, or carbohydrate expressed on the surface of cells, including specific cancer cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

A. Chimeric Antigen Receptors

In some embodiments, the CAR comprises: a) an intracellular signaling domain, b) a transmembrane domain, c) an extracellular domain comprising an antigen binding region, and, optionally d) one or more costimulatory domains.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acid polymers, including nucleic acid polymers encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acid polymers may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha or a synthetic molecule.

In some embodiments, the CAR nucleic acid vcomprises a partial or complete sequence encoding other costimulatory receptors either alone or in combination, such as a natural or modified extracellular domain, transmembrane domain and intracellular signaling domain of a specific molecule, such as CD28, for example. Other costimulatory domains include, but are not limited to one or more of CD28, CD27, OX-40 (CD134), ICOS, HVEM, GITR, LIGHT, CD40L, DR3, CD30, SLAM, CD2, CD226 (DNAM-1), MyD88, CD244, TMIGD2, BTNL3, NKG2D, DAP10, DAP12, 4-1BB (CD137), or a synthetic molecule. In addition to a primary signal initiated by CD3ζ, an additional signal provided by a costimulatory receptor inserted in a CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD2, CD3 zeta, CD3 epsilon, CD3 gamma, CD3 delta, CD45, CD4, CD5, CD8 (including CD8alpha), CD9, CD 16, CD22, CD33, CD37, CD64, CD80, CD86, CD 134, CD137, CD154, ICOS/CD278, GITR/CD357, NKG2D, PD-1, CTLA4, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

The hinge region of the CAR may be positioned N-terminal to the transmembrane domain and in some embodiments is derived either from a natural or from a synthetic source. A hinge sequence may also be referred to as a spacer or extracellular spacer and generally is the extracellular structural region of the CAR that separates the binding units from the transmembrane domain. In particular embodiments, the CAR comprises an immunoglobulin (Ig)-like domain hinges. The hinge generally supplies stability for efficient CAR expression and activity. The hinge may come from any suitable source, but in specific embodiments the hinge is from CD8a, CD28, PD-1, CTLA4, alpha, beta or zeta chain of the T-cell receptor, CD2, CD3 zeta, CD3 epsilon, CD3 gamma, CD3 delta, CD45, CD4, CD5, CD8b, CD9, CD16, CD22, CD27, CD32, CD33, CD37, CD64, CD80, CD86, CD134, CD137, CD154, CD160, BTLA, LAIR1, TIGIT, TIM4, ICOS/CD278, GITR/CD357, NKG2D, LAG-3, PD-L1, PD-1, TIM-3, HVEM, LIGHT, DR3, CD30, CD224, CD244, SLAM, CD226, DAP, or a combination thereof or others.

In certain embodiments, the platform technologies disclosed herein to genetically modify immune cells, such as T or NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CARP immune cells (Singh et al., 2008; Singh et al., 2011).

In certain embodiments, the cells are engineered to express a CD19-CAR sequence (SEQ ID NO:26) comprising the VH and VL of an anti-CD19 antibody, a fusion sequence of the CD8 hinge (any hinge may be referred to as a spacer or an extracellular spacer) and transmembrane regions, and the CD3 and CD28 signal transduction region.

(SEQ ID NO: 26)
ATGGCCCTGCCTGTGACAGCCCTGCTGCTGCCTCTGGCTCTGCTGCTGCA
TGCCGCTAGACCCGATATACAGATGACGCAGACAACGTCAAGTCTTTCCG
CCAGCTTGGGAGACCGAGTGACTATATCTTGTAGAGCAAGCCAGGATATT
TCTAAGTATCTTAACTGGTACCAACAAAAGCCCGATGGAACGGTTAAGCT
GCTTATATACCATACCAGTAGACTCCACTCCGGCGTACCATCACGGTTTT
CTGGCAGTGGCTCCGGGACCGACTATTCTTTGACGATCTCTAATCTCGAA
CAAGAGGATATTGCAACATACTTTTGTCAGCAAGGCAATACCTTGCCATA
TACGTTTGGGGGCGGGACAAAACTTGAGATAACCGGCGGCGGTGGTTCAG
GCGGTGGCGGTTCCGGTGGTGGGGGATCAGAGGTTAAGCTTCAGGAATCC
GGACCAGGTTTGGTTGCCCCCAGCCAATCTCTCAGCGTTACATGCACGGT
TTCAGGCGTCAGTCTCCCCGATTACGGTGTAAGTTGGATTCGGCAACCTC
CGCGAAAGGGTCTGGAATGGCTGGGGGTTATTTGGGGGAGTGAGACAACT
TATTACAACTCTGCACTTAAGAGTCGGCTTACCATCATCAAGGATAATTC
AAAATCACAAGTATTCCTGAAGATGAACTCATTGCAAACAGATGATACAG
CTATATACTATTGTGCCAAGCATTACTATTATGGTGGTTCTTATGCAATG
GATTACTGGGGGCAAGGCACGTCAGTGACAGTGAGTTCAACAACTACTCC
AGCACCACGACCACCAACACCTGCTCCAACTATCGCATCTCAACCACTTT
CTCTACGTCCAGAAGCATGCCGACCAGCTGCAGGAGGTGCAGTTCATACG
AGAGGTCTAGATTTCGCATGTGATATCTACATCTGGGCACCATTGGCTGG
GACTTGTGGTGTCCTTCTCCTATCACTGGTTATCACCCTTTACTGCTGGG
TTAGAAGTAAAAGAAGTAGGCTACTTCATAGTGATTACATGAATATGACT
CCTCGACGACCTGGTCCCACCCGTAAGCATTATCAGCCCTATGCACCACC
ACGAGATTTCGCAGCCTATCGCTCCAGAGTTAAATTTAGCAGAAGTGCAG
ATGCTCCTGCGTATAAACAGGGTCAAAACCAACTATATAATGAACTAAAT
CTAGGACGAAGAGAAGAATATGATGTTTTAGATAAAAGACGTGGTCGAGA
TCCTGAAATGGGAGGAAAACCTAGAAGAAAAAATCCTCAAGAAGGCCTAT
ATAATGAACTACAAAAAGATAAGATGGCAGAAGCTTATAGTGAAATTGGA
ATGAAAGGAGAACGTCGTAGAGGTAAAGGTCATGATGGTCTTTATCAAGG
TCTTAGTACAGCAACAAAAGATACATATGATGCACTTCATATGCAAGCAC
TTCCACCTCGTTTCGAAGAGCAAAAACTTATC

A specific example of a CAR (FMC63-CD8a hinge/TM-CD28-CD3z) that may be employed is as follows:

(SEQ ID NO: 27)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYIWAPLAGTCGVLLLSLVITLYCWVRSKRSRLLHSDYMNMT
PRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

FMC63-CD8a Hinge/TM-CD28-CD3z

One example of an anti-CD19 CAR is as follows that includes the anti-CD19 scFv FMC63, the CD8a hinge and transmembrane domain, CD28 costimulatory domain, and CD3zeta (FMC63-CD8a hinge/TM-CD28-CD3z):

(SEQ ID NO: 28)
ATGGCCCTGCCAGTGACCGCCCTGCTGCTGCCACTGGCACTGCTGCTGCA
CGCAGCAAGGCCAGACATCCAGATGACACAGACCACAAGCTCCCTGTCCG
CCTCTCTGGGCGACAGAGTGACCATCTCTTGCAGGGCCAGCCAGGATATC
TCCAAGTATCTGAATTGGTACCAGCAGAAGCCTGATGGCACAGTGAAGCT
GCTGATCTATCACACCTCTAGACTGCACAGCGGCGTGCCATCCAGGTTTA
GCGGCTCCGGCTCTGGCACAGACTACTCTCTGACCATCAGCAATCTGGAG
CAGGAGGATATCGCCACCTATTTCTGCCAGCAGGGCAACACACTGCCTTA
CACCTTTGGCGGCGGCACAAAGCTGGAGATCACCGGCGGCGGCGGCTCTG
GAGGAGGAGGAAGCGGAGGAGGAGGATCCGAGGTGAAGCTGCAGGAGAGC
GGACCAGGACTGGTGGCACCCAGCCAGTCCCTGTCTGTGACATGTACCGT
GTCCGGCGTGTCTCTGCCAGACTACGGCGTGAGCTGGATCAGACAGCCAC
CTAGGAAGGGACTGGAGTGGCTGGGCGTGATCTGGGGCTCCGAGACCACA
TACTATAACTCCGCCCTGAAGTCTCGGCTGACCATCATCAAGGACAACAG
CAAGTCCCAGGTGTTTCTGAAGATGAATTCCCTGCAGACAGACGATACCG
CCATCTACTATTGCGCCAAGCACTACTATTACGGCGGCTCTTATGCCATG
GATTACTGGGGCCAGGGCACAAGCGTGACCGTGTCTAGCACCACAACCCC
TGCACCAAGACCACCAACACCAGCACCTACCATCGCAAGCCAGCCTCTGT
CCCTGAGGCCAGAGGCATGCAGGCCAGCAGCAGGAGGAGCAGTGCACACC
AGGGGCCTGGACTTCGCCTGCGATATCTACATCTGGGCACCACTGGCAGG
AACATGTGGAGTGCTGCTGCTGTCTCTGGTCATCACCCTGTATTGTTGGG
TGAGAAGCAAGAGATCCAGGCTGCTGCACAGCGACTACATGAATATGACA
CCAAGGAGACCAGGACCAACCAGGAAGCACTATCAGCCTTACGCACCTCC
AAGGGACTTCGCAGCATATAGGAGCAGGGTGAAGTTTTCTCGCAGCGCCG
ATGCCCCAGCCTATcAGCAGGGCCAGAACCAGCTGTACAACGAGCTGAAT
CTGGGCAGGCGCGAGGAGTACGACGTGCTGGATAAGAGGAGAGGAAGGGA
TCCAGAGATGGGAGGCAAGCCTAGGCGCAAGAACCCACAGGAGGGCCTGT
ATAATGAGCTGCAGAAGGACAAGATGGCCGAGGCCTACAGCGAGATCGGC
ATGAAGGGAGAGAGGAGAAGGGGCAAGGGACACGATGGCCTGTATCAGGG
CCTGTCCACAGCCACCAAGGACACCTACGATGCACTGCACATGCAGGCAC
TGCCACCTAGA

In the example of SEQ ID NO:28, the following components of the CAR are delineated as follows:

CD8 signal peptide
(SEQ ID NO: 29)
ATGGCCCTGCCAGTGACCGCCCTGCTGCTGCCACTGGCACTGCTGCTGCA
CGCAGCAAGGCCA
FMC63 light chain
(SEQ ID NO: 30)
GACATCCAGATGACACAGACCACAAGCTCCCTGTCCGCCTCTCTGGGCGA
CAGAGTGACCATCTCTTGCAGGGCCAGCCAGGATATCTCCAAGTATCTGA
ATTGGTACCAGCAGAAGCCTGATGGCACAGTGAAGCTGCTGATCTATCAC
ACCTCTAGACTGCACAGCGGCGTGCCATCCAGGTTTAGCGGCTCCGGCTC
TGGCACAGACTACTCTCTGACCATCAGCAATCTGGAGCAGGAGGATATCG
CCACCTATTTCTGCCAGCAGGGCAACACACTGCCTTACACCTTTGGCGGC
GGCACAAAGCTGGAGATCACC
Linker
(SEQ ID NO: 31)
GGCGGCGGCGGCTCTGGAGGAGGAGGAAGCGGAGGAGGAGGATCC
Heavy chain
(SEQ ID NO: 32)
GAGGTGAAGCTGCAGGAGAGCGGACCAGGACTGGTGGCACCCAGCCAGTC
CCTGTCTGTGACATGTACCGTGTCCGGCGTGTCTCTGCCAGACTACGGCG
TGAGCTGGATCAGACAGCCACCTAGGAAGGGACTGGAGTGGCTGGGCGTG
ATCTGGGGCTCCGAGACCACATACTATAACTCCGCCCTGAAGTCTCGGCT
GACCATCATCAAGGACAACAGCAAGTCCCAGGTGTTTCTGAAGATGAATT
CCCTGCAGACAGACGATACCGCCATCTACTATTGCGCCAAGCACTACTAT
TACGGCGGCTCTTATGCCATGGATTACTGGGGCCAGGGCACAAGCGTGAC
CGTGTCTAGC
CD8a hinge
(SEQ ID NO: 33)
ACCACAACCCCTGCACCAAGACCACCAACACCAGCACCTACCATCGCAAG
CCAGCCTCTGTCCCTGAGGCCAGAGGCATGCAGGCCAGCAGCAGGAGGAG
CAGTGCACACCAGGGGCCTGGACTTCGCCTGCGAT
CD8TM
(SEQ ID NO: 34)
ATCTACATCTGGGCACCACTGGCAGGAACATGTGGAGTGCTGCTGCTGTC
TCTGGTCATCACCCTGTATTGTTGGGTG
CD28 Costimulatory Domain
(SEQ ID NO: 35)
AGAAGCAAGAGATCCAGGCTGCTGCACAGCGACTACATGAATATGACACC
AAGGAGACCAGGACCAACCAGGAAGCACTATCAGCCTTACGCACCTCCAA
GGGACTTCGCAGCATATAGGAGC
CD3 zeta
(SEQ ID NO: 36)
AGGGTGAAGTTTTCTCGCAGCGCCGATGCCCCAGCCTATcAGCAGGGCCA
GAACCAGCTGTACAACGAGCTGAATCTGGGCAGGCGCGAGGAGTACGACG
TGCTGGATAAGAGGAGAGGAAGGGATCCAGAGATGGGAGGCAAGCCTAGG
CGCAAGAACCCACAGGAGGGCCTGTATAATGAGCTGCAGAAGGACAAGAT
GGCCGAGGCCTACAGCGAGATCGGCATGAAGGGAGAGAGGAGAAGGGGCA
AGGGACACGATGGCCTGTATCAGGGCCTGTCCACAGCCACCAAGGACACC
TACGATGCACTGCACATGCAGGCACTGCCACCTAGA

The corresponding amino acid sequence of FMC63-CD8a hinge/TM-CD28-CD3z is as follows:

(SEQ ID NO: 37)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYIWAPLAGTCGVLLLSLVITLYCWVRSKRSRLLHSDYMNMT
PRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

In the example of SEQ ID NO:37, the following components of the CAR are delineated as follows:

CD8 signal peptide
(SEQ ID NO: 38)
MALPVTALLLPLALLLHAARP
FMC63 light chain
(SEQ ID NO: 39)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT (bolded letters are CDRs)
Linker
(SEQ ID NO: 40)
GGGGSGGGGSGGGGS
Heavy chain
(SEQ ID NO: 41)
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSS (bolded letters are CDRs)
CD8a hinge
(SEQ ID NO: 42)
TTTPAPRPPTPAPTIASQPLSLRPEACRPAAGGAVHTRGLDFACD
CD8
(SEQ ID NO: 43)
TMIYIWAPLAGTCGVLLLSLVITLYCWV
CD28 Costimulatory Domain
(SEQ ID NO: 44)
RSKRSRLLHSDYMNMTPRRPGPTRKHYQPYAPPRDFAAYRS
CD3 zeta
(SEQ ID NO: 45)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT

YDALHMQALPPR

FMC63-CD28 Hinge/TM-CD28-CD3z

One example of an anti-CD19 CAR is as follows that includes the anti-CD19 scFv FMC63, the CD28 hinge and transmembrane domain, CD28 costimulatory domain, and CD3zeta (FMC63-CD28 hinge/TM-CD28-CD3z):

(SEQ ID NO: 46)
ATGCTGCTGCTCGTGACCTCCCTGCTGCTGTGCGAGCTGCCACACCCTGC
CTTCCTGCTGATCCCTGACATCCAGATGACCCAGACCACAAGCTCCCTGT
CCGCCTCTCTGGGCGACAGAGTGACAATCTCTTGTAGGGCCAGCCAGGAT
ATCTCCAAGTATCTGAACTGGTACCAGCAGAAGCCAGATGGCACCGTGAA
GCTGCTGATCTATCACACATCTAGGCTGCACAGCGGAGTGCCATCCCGGT
TTAGCGGATCCGGATCTGGAACCGACTACTCTCTGACAATCAGCAACCTG
GAGCAGGAGGATATCGCCACCTATTTCTGCCAGCAGGGCAATACCCTGCC
TTACACATTTGGCGGCGGCACAAAGCTGGAGATCACCGGCAGCACATCCG
GATCTGGCAAGCCAGGATCCGGAGAGGGATCTACCAAGGGAGAGGTGAAG
CTGCAGGAGAGCGGACCAGGACTGGTGGCACCCAGCCAGTCCCTGTCTGT
GACCTGTACAGTGTCCGGCGTGTCTCTGCCAGACTACGGCGTGAGCTGGA
TCAGGCAGCCACCTAGGAAGGGACTGGAGTGGCTGGGCGTGATCTGGGGC
TCCGAGACCACATACTATAATAGCGCCCTGAAGTCCAGACTGACCATCAT
CAAGGATAACAGCAAGTCCCAGGTGTTCCTGAAGATGAATTCCCTGCAGA
CCGACGATACAGCCATCTACTATTGCGCCAAGCACTACTATTACGGCGGC
TCCTATGCCATGGACTACTGGGGCCAGGGCACCTCTGTGACAGTGTCTAG
CGCCGCCGCCATCGAAGTGATGTATCCACCCCCTTACCTGGATAACGAGA
AGAGCAATGGCACCATCATCCACGTGAAGGGCAAGCACCTGTGCCCATCT
CCCCTGTTCCCTGGCCCAAGCAAGCCCTTTTGGGTGCTGGTGGTGGTGGG
AGGCGTGCTGGCCTGTTATTCTCTGCTGGTGACAGTGGCCTTCATCATCT
TTTGGGTGAGGAGCAAGCGGAGCAGGCTGCTGCACAGCGACTACATGAAC
ATGACCCCCCGGAGACCCGGCCCTACAAGAAAGCACTATCAGCCTTACGC
ACCACCAAGGGACTTCGCAGCCTATAGAAGCAGGGTGAAGTTTTCTCGCA
GCGCCGATGCACCAGCATATCAGCAGGGACAGAATCAGCTGTACAACGAG
CTGAATCTGGGCAGGCGCGAGGAGTACGACGTGCTGGATAAGAGGAGAGG
AAGGGATCCTGAGATGGGAGGCAAGCCTAGGCGCAAGAACCCACAGGAGG
GCCTGTATAATGAGCTGCAGAAGGACAAGATGGCCGAGGCCTACTCCGAG
ATCGGCATGAAGGGAGAGCGGAGAAGGGGCAAGGGACACGATGGCCTGTA
TCAGGGCCTGTCTACCGCCACAAAGGACACCTACGATGCCCTGCACATGC
AGGCCCTGCCTCCACGG

An amino acid sequence of FMC63-CD28 hinge/TM-CD28-CD3z is as follows:

(SEQ ID NO: 47)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR

Another example of nucleic acid sequence for FMC63-CD28 hinge-TM CAR is as follows:

(SEQ ID NO: 48)
ATGCTGCTGCTCGTGACCTCCCTGCTGCTGTGCGAGCTGCCACACCCTGC
CTTCCTGCTGATCCCTGACATCCAGATGACCCAGACCACAAGCTCCCTGT
CCGCCTCTCTGGGCGACAGAGTGACAATCTCTTGTAGGGCCAGCCAGGAT
ATCTCCAAGTATCTGAACTGGTACCAGCAGAAGCCAGATGGCACCGTGAA
GCTGCTGATCTATCACACATCTAGGCTGCACAGCGGAGTGCCATCCCGGT
TTAGCGGATCCGGATCTGGAACCGACTACTCTCTGACAATCAGCAACCTG
GAGCAGGAGGATATCGCCACCTATTTCTGCCAGCAGGGCAATACCCTGCC
TTACACATTTGGCGGCGGCACAAAGCTGGAGATCACCGGCAGCACATCCG
GATCTGGCAAGCCAGGATCCGGAGAGGGATCTACCAAGGGAGAGGTGAAG
CTGCAGGAGAGCGGACCAGGACTGGTGGCACCCAGCCAGTCCCTGTCTGT
GACCTGTACAGTGTCCGGCGTGTCTCTGCCAGACTACGGCGTGAGCTGGA
TCAGGCAGCCACCTAGGAAGGGACTGGAGTGGCTGGGCGTGATCTGGGGC
TCCGAGACCACATACTATAATAGCGCCCTGAAGTCCAGACTGACCATCAT
CAAGGATAACAGCAAGTCCCAGGTGTTCCTGAAGATGAATTCCCTGCAGA
CCGACGATACAGCCATCTACTATTGCGCCAAGCACTACTATTACGGCGGC
TCCTATGCCATGGACTACTGGGGCCAGGGCACCTCTGTGACAGTGTCTAG
CATCGAAGTGATGTATCCACCCCCTTACCTGGATAACGAGAAGAGCAATG
GCACCATCATCCACGTGAAGGGCAAGCACCTGTGCCCATCTCCCCTGTTC
CCTGGCCCAAGCAAGCCCTTTTGGGTGCTGGTGGTGGTGGGAGGCGTGCT
GGCCTGTTATTCTCTGCTGGTGACAGTGGCCTTCATCATCTTTTGGGTGA
GGAGCAAGCGGAGCAGGCTGCTGCACAGCGACTACATGAACATGACCCCC
CGGAGACCCGGCCCTACAAGAAAGCACTATCAGCCTTACGCACCACCAAG
GGACTTCGCAGCCTATAGAAGCAGGGTGAAGTTTTCTCGCAGCGCCGATG
CACCAGCATATCAGCAGGGACAGAATCAGCTGTACAACGAGCTGAATCTG
GGCAGGCGCGAGGAGTACGACGTGCTGGATAAGAGGAGAGGAAGGGATCC
TGAGATGGGAGGCAAGCCTAGGCGCAAGAACCCACAGGAGGGCCTGTATA
ATGAGCTGCAGAAGGACAAGATGGCCGAGGCCTACTCCGAGATCGGCATG
AAGGGAGAGCGGAGAAGGGGCAAGGGACACGATGGCCTGTATCAGGGCCT
GTCTACCGCCACAAAGGACACCTACGATGCCCTGCACATGCAGGCCCTGC
CTCCACGG
CD28 hinge:
(SEQ ID NO: 49)
IEVMYPPPYLDNEKSNGTIIHVKGKHLCPSPLFPGPSKP
CD28 hinge nucleic acid sequence
(SEQ ID NO: 50)
ATCGAAGTGATGTATCCACCCCCTTACCTGGATAACGAGAAGAGCAATGG
CACCATCATCCACGTGAAGGGCAAGCACCTGTGCCCATCTCCCCTGTTCC
CTGGCCCAAGCAAGCCC
CD28 TM domain
(SEQ ID NO: 51)
FWVLVVVGGVLACYSLLVTVAFIIFWV

FMC63-PD-1 Hinge-TM CAR

An example of a CAR having the following components is CSF2RA signal peptide-FMC63 light chain-Linker-Heavy chain-PD1 hinge-PD-1TM-CD28 Costim-CD3zeta is as follows:

(SEQ ID NO: 52)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSW1RQPPRKGLEWLGVIWG
SETTYYNSALKSRLTIIKDNSKSQVFLKMNSLQTDDTAIYYCAKHYYYGG
SYAMDYWGQGTSVTVSSQVPTAHPSPSPRPAGQFQTLVVGVVGGLLGSLV
LLVWVLAVIERSKRSRLLHSDYMNMTPRRPGPTRKHYQPYAPPRDFAAYR
SRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR

The nucleic acid sequence for FMC63-PD-1 hinge-TM CAR is as follows:

(SEQ ID NO: 53)
ATGCTACTGCTGGTGACCAGCCTCCTGCTGTGCGAGCTGCCCCACCCCGC
GTTCCTGCTCATCCCCGACATCCAGATGACCCAGACGACCTCCTCGCTGA
GTGCATCACTGGGAGACCGCGTCACCATCTCATGCCGAGCTTCCCAGGAC
ATTTCCAAGTACCTGAACTGGTACCAGCAGAAGCCTGACGGCACCGTCAA
GCTGCTTATCTACCACACTAGTCGCCTCCACTCTGGCGTGCCCTCTAGAT
TTAGTGGCTCCGGCTCGGGCACCGACTACAGCCTGACCATCAGCAACCTG
GAACAGGAGGACATAGCCACTTACTTCTGCCAGCAGGGCAACACCCTGCC
CTATACCTTCGGCGGCGGCACCAAGCTGGAGATCACGGGTTCGACCTCCG
GATCTGGGAAGCCGGGGTCCGGAGAGGGCTCCACTAAGGGTGAGGTGAAG
CTCCAGGAGAGCGGGCCTGGGCTGGTAGCGCCCAGCCAGAGCTTATCCGT
GACCTGTACCGTGTCGGGAGTCTCGCTGCCTGATTACGGCGTGAGCTGGA
TTCGCCAGCCGCCCCGCAAAGGCTTGGAATGGCTAGGTGTGATCTGGGGC
TCCGAGACCACCTATTACAACTCCGCCCTGAAGTCCCGGCTTACGATCAT
CAAGGACAACTCCAAGTCTCAGGTGTTCTTGAAGATGAACTCTCTTCAAA
CAGATGACACCGCCATCTATTACTGTGCCAAGCACTACTACTACGGCGGC
AGCTACGCCATGGATTATTGGGGCCAAGGAACTTCTGTTACAGTTTCCTC
TCAGGTCCCAACAGCGCATCCCTCTCCAAGCCCGCGTCCCGCTGGACAGT
TCCAGACTCTGGTGGTGGGCGTGGTGGGCGGGCTGCTGGGTTCTTTGGTG
CTGCTGGTGTGGGTCCTCGCTGTCATTGAGCGCAGCAAGCGCAGCCGCCT
GTTGCACAGCGATTACATGAATATGACTCCGCGCCGGCCTGGCCCAACGC
GTAAGCACTACCAGCCGTACGCGCCCCCGAGAGACTTCGCTGCATACAGG
TCCCGCGTAAAATTTTCGCGCTCTGCGGACGCTCCTGCCTATCAGCAGGG
TCAGAACCAGCTGTACAATGAGCTCAACCTGGGCCGTAGGGAGGAGTACG
ATGTGCTCGACAAACGCCGTGGTCGGGACCCGGAGATGGGCGGTAAACCT
CGGCGCAAGAATCCTCAGGAGGGCCTTTACAACGAGCTGCAGAAGGACAA
AATGGCCGAGGCCTACTCCGAGATCGGTATGAAGGGGGAACGCCGTCGCG
GCAAGGGCCACGATGGATTGTATCAGGGCCTGTCCACCGCCACCAAGGAC
ACCTACGACGCCCTGCATATGCAGGCCTTGCCGCCCCGC
PD-1 hinge
(SEQ ID NO: 54)
QVPTAHPSPSPRPAGQFQTLV
PD-1 TM domain
(SEQ ID NO: 55)
VGVVGGLLGSLVLLVWVLAVI

FMC63-CTLA4 Hinge-TM CAR:

CSF2RA signal peptide-FMC63 light chain-Linker-Heavy chain-CTLA4 hinge-CTLA-4 TM-CD28 Cost-CD3zeta

(SEQ ID NO: 56)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSvidpepcpdsdfllwilaayssglffysfllta
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
(SEQ ID NO: 57)
ATGTTACTGCTCGTTACTTCGCTGCTGCTGTGCGAGCTGCCACACCCCGC
GTTCTTGCTGATTCCGGATATCCAGATGACCCAGACGACCTCCTCCCTCT
CCGCTAGTCTGGGGGACCGCGTGACCATCTCATGCCGAGCTTCCCAGGAC
ATCTCTAAGTACCTGAACTGGTACCAACAGAAGCCCGATGGGACCGTGAA
GTTGCTCATTTACCACACCTCTCGTCTACACAGTGGTGTCCCTTCTCGCT
TCTCGGGATCCGGTTCTGGTACAGATTACTCCTTGACCATCTCAAATCTT
GAACAGGAGGACATCGCCACTTATTTCTGTCAGCAGGGCAACACGCTTCC
GTACACCTTCGGCGGCGGTACTAAGCTGGAGATCACCGGCTCGACCAGCG
GCTCGGGCAAGCCCGGCTCCGGCGAAGGCAGCACCAAGGGCGAGGTGAAG
CTCCAGGAGAGCGGACCCGGACTGGTGGCGCCAAGCCAGAGCCTGTCTGT
GACCTGCACCGTGTCCGGCGTATCTCTGCCCGACTACGGCGTTAGTTGGA
TCCGCCAGCCGCCCCGCAAAGGCCTGGAGTGGCTAGGGGTCATATGGGGC
TCCGAGACCACATACTACAACAGCGCACTGAAATCCCGCTTGACCATCAT
CAAGGACAACAGCAAGAGCCAGGTGTTCCTGAAGATGAATTCCTTGCAGA
CTGATGACACCGCCATCTATTACTGTGCTAAGCACTATTACTACGGTGGC
AGCTACGCGATGGATTATTGGGGCCAGGGAACTTCTGTGACGGTGTCCTC
CGTGATTGACCCGGAGCCATGTCCTGACAGTGACTTCCTGCTTTGGATCC
TGGCCGCTGTCTCTTCTGGCCTTTTCTTTTACTCCTTCCTGCTGACAGCC
AGGAGCAAGCGCAGCCGCCTGTTGCACTCCGACTACATGAACATGACTCC
TCGCCGCCCCGGGCCAACCCGCAAGCACTACCAACCCTATGCTCCCCCGC
GCGACTTTGCGGCCTACAGATCACGAGTCAAATTTAGCCGCTCGGCGGAC
GCTCCTGCCTACCAGCAGGGACAGAACCAGCTTTACAACGAGCTCAACCT
GGGCAGAAGGGAGGAGTACGATGTGCTGGACAAGCGTCGCGGCCGGGACC
CCGAGATGGGCGGTAAGCCTCGGCGCAAGAACCCTCAGGAGGGCCTGTAC
AACGAGCTGCAGAAGGACAAAATGGCCGAGGCTTATTCGGAAATCGGTAT
GAAGGGGGAGCGGCGTCGTGGCAAAGGTCATGACGGCCTCTACCAGGGGC
TGTCCACCGCCACCAAAGATACCTACGACGCATTACATATGCAGGCCCTG
CCGCCGAGG
CSF2RA signal peptide
(SEQ ID NO: 58)
MLLLVTSLLLCELPHPAFLLIP
CTLA4 hinge
(SEQ ID NO: 59)
VIDPEPCPDSD
CTLA4 TM domain
(SEQ ID NO: 60)
FLLWILAAVSSGLFFYSFLLT

B. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form. In alternative embodiments, the cells lack an engineered TCR; for example, endogenous TCR in the cells may target cancer or infectious diseases (e.g., CMV or EBV-specific T cells with endogenous TCR).

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid polymer encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

C. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

D. Antigens

Among the antigens targeted by the genetically engineered antigen receptors or by naturally expressed antigen receptors (e.g., TCR) on infinite immune cells are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, CD20, CD22, CD30, CD70, CD79a, CD79b, SLAM-F7NY-ESO, EGFRvIII, Muc-1, Her2, CA-125, WT-1, Mage-A3, Mage-A4, Mage-A10, TRAIL/DR4, CEA. In particular aspects, the antigens for the one or two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alphafetoprotein.

In certain embodiments, the antigen may be microbial. In some embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, coronaviruses such as SARS-CoV, SARS-CoV-2, or MERS, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

E. Suicide Genes

The infinite immune cells of the present disclosure (including those that may express one or more CARS and/or one or more engineered TCRs) may comprise one or more suicide genes. The term “suicide gene” as used herein is defined as a gene which, upon administration of a prodrug, effects transition of a gene product to a compound which kills its host cell. Examples of suicide gene/prodrug combinations which may be used are truncated EGFR and cetuximab; Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### V. METHODS OF DELIVERY TO THE CELLS

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

A. Viral Vectors

Viral vectors encoding BCL6 and a cell survival-promoting gene and/or an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid polymer and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acid polymer s into cells (e.g., mammalian cells). Non-limiting examples of virus vectors that may be used to deliver a nucleic acid polymer of certain aspects of the present disclosure are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid polymer sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

B. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

C. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

D. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

E. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

F. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

G. Methods of Nucleic Acid Polymer Delivery

The engineered immune cells may be constructed using any of the many well-established gene transfer methods known to those skilled in the art. In certain embodiments, the engineered cells are constructed using viral vector-based gene transfer methods to introduce nucleic acid polymers. The viral vector-based gene transfer method may comprise a lentiviral vector, a retroviral vector, an adenoviral or an adeno-associated viral vector. In certain embodiments, the engineered cells are constructed using non-viral vector-based gene transfer methods to introduce nucleic acid polymers. In certain embodiments, the non-viral vector-based gene transfer method comprises a gene-editing method selected from the group consisting of a zinc-finger nuclease (ZFN), a transcription activator-like effector nuclease (TALENs), and a clustered regularly interspaced short palindromic repeats (CRISPR)/CRISPR-associated protein 9 (Cas9) nuclease. In certain embodiments, the non-viral vector-based gene editing method comprises a transfection or transformation method selected from the group consisting of lipofection, nucleofection, virosomes, liposomes, polycation or lipid:nucleic acid conjugates, naked DNA, artificial virions, and agent-enhanced uptake of DNA.

The cells may be engineered to express the gene(s) of interest and/or antigen receptor by random insertion or site-directed insertion, such as by gene editing methods including but not limited to meganucleases, zinc finger nucleases (ZFNs), transcription activator-like effector-based nucleases (TALEN), and the CRISPR-Cas system.

In addition to viral delivery of the nucleic acid polymers encoding the gene(s) of interest and/or antigen receptor, the following are additional methods of recombinant gene delivery to a given host cell and are thus considered in the present disclosure. Introduction of a nucleic acid polymer, such as DNA or RNA, into the immune cells of the current disclosure may use any suitable methods for nucleic acid polymer delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### VI. Methods of Treatment

The present infinite immune cells may be used in both therapy and research. The present infinite immune cells, including T cells or NK cells that express CARs and/or engineered TCRs, may be used to treat cancer, infectious disease, an immune disorder, or an inflammatory disorder.

In one method, allogeneic off-the-shelf CAR T cells targeting antigens such as CD19, CD20, CD22, CD79a, CD79b, or BAFF-R may be used to treat B cell leukemias and lymphoma either alone or in combination. Allogeneic off-the-shelf anti-mesothelin CAR T cells may be used to treat mesothelioma, pancreatic adenocarcinoma, or ovarian cancer, as one example. NY-ESO targeted TCR-T cells may be used to treat melanoma or multiple myeloma, as one example. Virus-specific T cells against viruses such as EBV, CMV, BK virus, etc., may be used to treat the respective viral infections. Allogeneic inhibitory or regulatory T cells may be used to treat autoimmune disorders, GVHD, and other inflammatory disorders.

Gamma/delta T cells and viral specific T cells are unlikely to cause GvHD but provide additional anti-tumor and/or anti-viral functions, in specific embodiments. In specific embodiments, viral-specific infinite T cells can be utilized for at least two purposes. First, viral-specific infinite T cells may be used to treat a particular viral infection, such as CMV or EBV infection, or certain cancers. A second embodiment is to transduce one or more CARs and/or engineered TCRs into viral-specific T cells. Such infinite CAR T cells with viral-specific endogenous TCR may have potential advantages, such as being unlikely to cause GVHD. Such viral-specific endogenous TCR-bearing cells do not require gene editing methods to knock out TCR in the T cells. If one combines gene editing technology such as CRISPR/Cas9, viral-specific T cells are not necessarily needed to produce CAR-T cells. Alternatively, one can utilize gamma/delta infinite CAR T cells or CAR-NK or CAR-NKT or CAR-innate lymphoid cells, which do not cause GvHD and are not expected to need TCR knock-out.

When intended for use in humans, the modified cell lines of the present invention are first tested for tumoricidal activity and therapeutic efficacy in animal models, such as the NSG mouse models commonly used in cancer research. Such studies in mice are preclinical studies that can be performed before therapeutic usage in patients is undertaken.

The infinite immune cells may be used for treating cancers, including hematological and non-hematological malignancies, such as by administering to a patient an effective amount of modified cytotoxic infinite T cells expressing different CARs or TCRs against different tumor targets either alone or in combination. For example, CD19inCARTs, one of which is Ie1-L4aJ3 cells (CD8 positive cells from healthy donor 1 transduced with a CAR against human CD19 with truncated human EFGR marker), may be administered together with IL-2 or IL-15 for treatment of patients with B cell leukemias or lymphomas. The Ie1-L4aJ3 cells may be present in a conventional pharmaceutical excipient, such as water or buffered saline. Upon administration to the patient, the modified cells can arrest the growth of tumor by CD19-directed killing. For human patients, the immune cells may be given by intravenous infusion (i.v.). However, other methods of administration, such as subcutaneous (s.c.) injection may be utilized. Upon successful eradication of the neoplastic cells, the immune cells can be cleared by withdrawal of IL-2 or IL-15 or by infusion of anti-EGFR antibody.

Appropriate dosages of the infinite immune cells (and one or more cytokines, such as IL-2 and/or IL-15, when used) vary depending upon the age, health, sex, and weight of the recipient, as well as any other concurrent treatments the recipient is undergoing for related or non-related conditions. One of skill in the art can readily determine the appropriate dose of the modified cells and drug to be administered to the patient, depending on the above-mentioned factors. The number of cells that constitute an effective tumoricidal amount can be determined using animal models. These parameters can be readily determined by one of skill in the art.

The effectiveness of the present therapy against tumors may be determined by detection of any surviving tumor cells in samples of the patient's peripheral blood or bone marrow, or by other diagnostic imaging studies such as CT, MRI or PET scan. Similarly, any residual, unwanted modified infinite T cells may be monitored using methods such as flow cytometry and polymerase chain reaction.

Compared to previous cytotoxic cell lines such as TALL-104 and NK-92 cells, infinite immune cells are generated from normal immune cells. Therefore, the leukemogenic risk is low with infinite immune cells compared with TALL-104 and NK-92 as the former are not expected to have any other unknown tumorigenic genetic mutations. Moreover, the proliferation of the infinite cells can be stopped by discontinuation of IL-2 or IL-15. This is an unrivalled safety advantage over the leukemia-derived cell lines, TALL-104 and NK-92.

In some embodiments, the present disclosure provides methods for immunotherapy comprising administering an effective amount of the immune cells of the present disclosure. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a growth or differentiation factor that promotes the growth, differentiation, and activation of the immune cells is administered to the subject either concomitantly with the immune cells or subsequently to the immune cells. The immune cell growth factor can be any suitable growth factor that promotes the growth and activation of the immune cells. Examples of suitable immune cell growth or differentiation factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, intraventricular, intrathecal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, monoclonal antibodies such as rituximab, trastuzumab, etc, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

A. Pharmaceutical Compositions

Also provided herein are pharmaceutical compositions and formulations comprising infinite immune cells (e.g., T cells, or NK cells) and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, targeted therapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods and compositions of the disclosure. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells, NKT cells, innate lymphoid cells, and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. ARTICLES OF MANUFACTURE OR KITS

An article of manufacture or a kit is provided comprising infinite immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1— Infinite Immune Cells for Adoptive Therapy

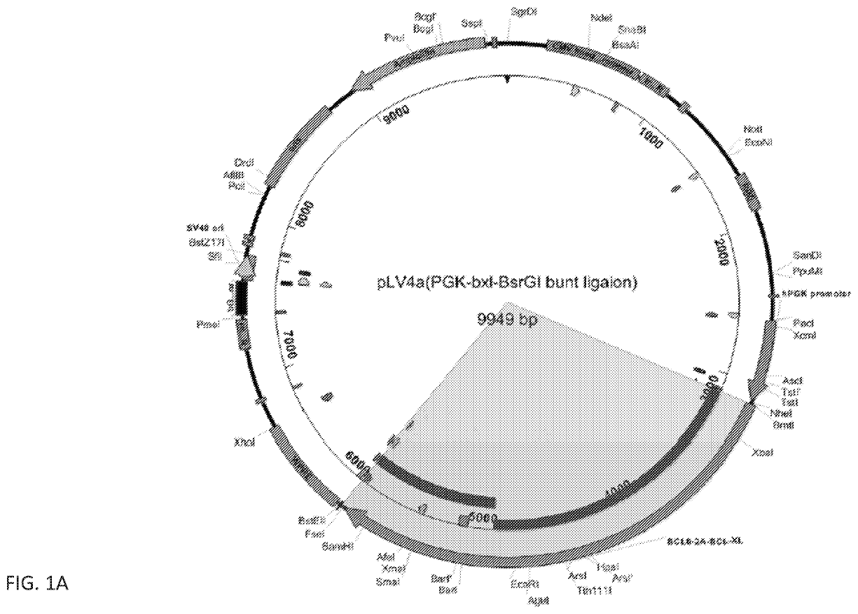

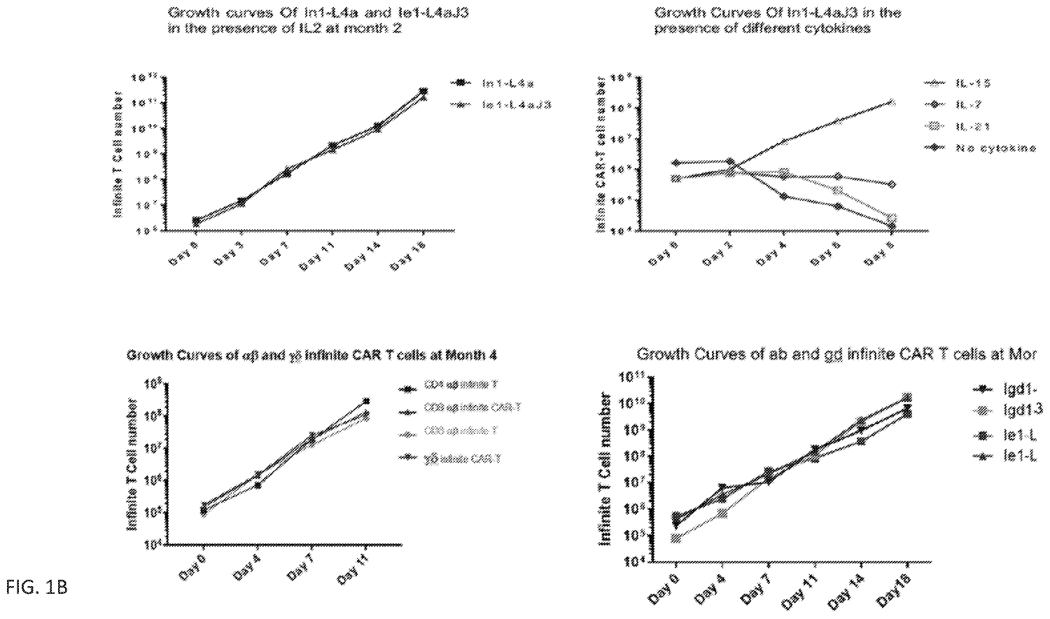

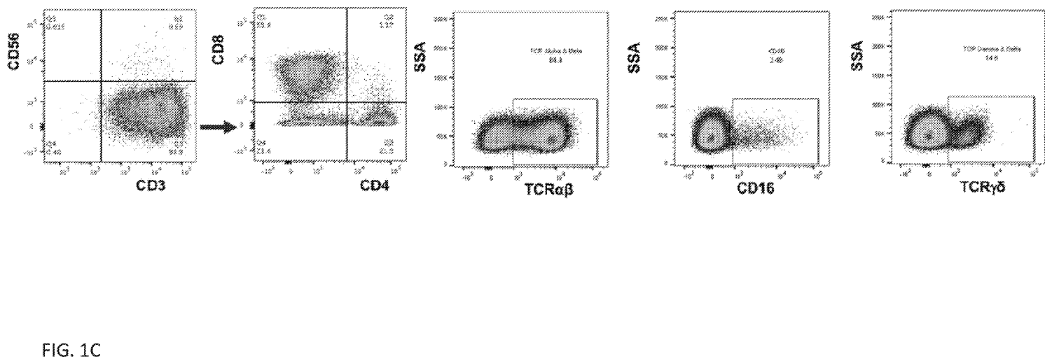

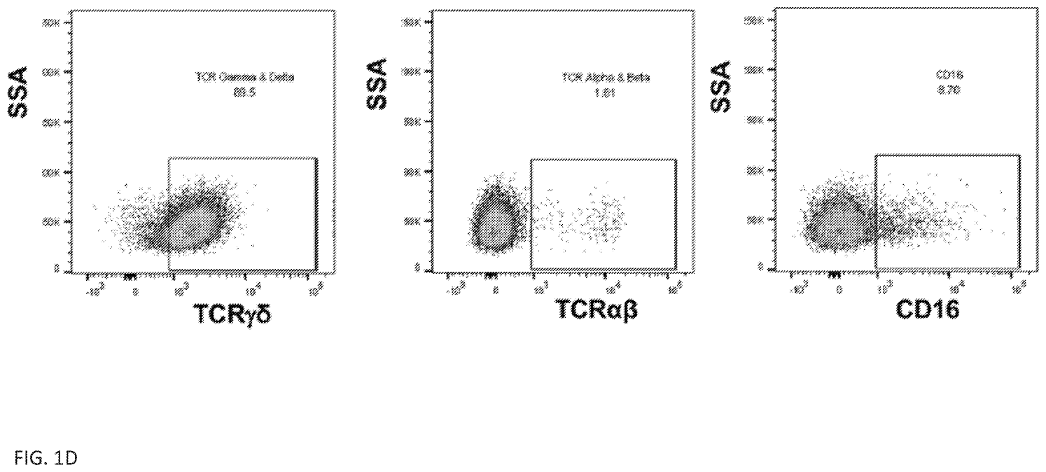

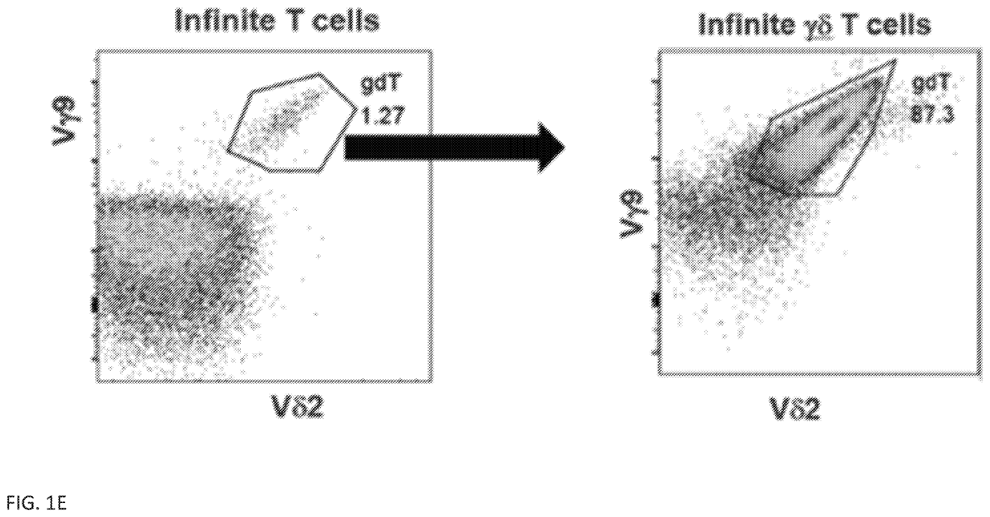

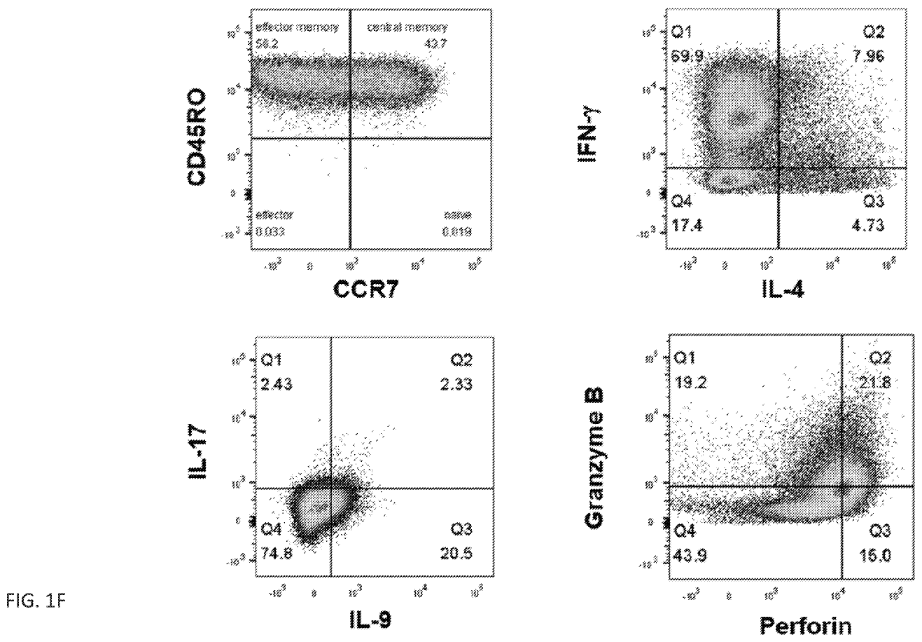

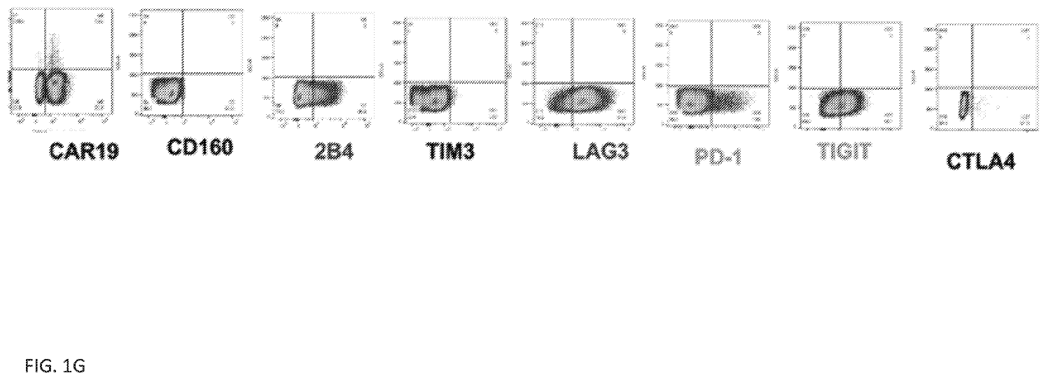

293T cells were cultured and passaged in a T75 flask in 10 mL high glucose DMEM medium with 10% FBS and 1% Pen/Strep. Once the 293T cells reached 90% confluency, they were used for transfection next day for lentiviral vector generation and packaging of plasmids. the coding sequences of BCL6 and Bcl-xL genes can be joined with a T2A sequence to generate one open reading frame which can express BCL6 and Bcl-xL genes simultaneously. This BCL6-T2A-Bcl-xL open reading frame may be cloned into a lentiviral vector using Gibson assembly following the protocol provided by NEB. The final vector was designated as pLV4a plasmid (FIG. 1A). This pLV4a plasmid was co-transfected into 293T cells with a lentiviral vector packaging mixture from abm company. Viral supernatant was concentrated using Lenti-X concentrator from Clontech.

For the development of the infinite cell lines from a healthy donor, normal T cells were isolated from a healthy donor using RosetteSep™ Human T Cell Enrichment Cocktail and SepMate™-50 tubes from STEMCELL Technologies. The isolated T cells were then cultured with RPMI-1640 medium (Gibco) supplemented with 10% FBS, 2% HEPES, 1% sodium pyruvate, and 0.01% 2-mercaptoethanol and 50-1000 IU/mL IL-2 (Genscript) and 25 μL/mL ImmunoCult™ human CD3/CD28/CD2 T cell activator (STEMCELL Technologies). After 36-48 hours culture, one million cultured T cells were transduced with the concentrated pLV4a lentiviral vector (FIG. 1A) in the presence of RetroNectin (Clontech), then the T cells were cultured in RPMI1640 medium in the presence of 50-1000 IU/mL of IL-2, subcultured and split when necessary. Some transduced T cells continued to proliferate indefinitely. This method generated a T cell line referred to as ‘infinite T cells’ from healthy donor T cells, which proliferate in the presence of recombinant human IL-2 or IL-15.

Next, several novel infinite T cell lines were generated by the above methods. They were designated as In1-L4a T cells which consists of multiple subsets of T cells. A series of T cells were isolated and generated using In1-L4a T cells by cell sorting or gene engineering, including the Ie1-L4a, If1-L4a, In1-L4aJ3, Ie1-L4aJ3, Igd1-L4a, Igd1-L4aJ3, etc. A detailed description of these IL-2 or IL-15 dependent infinite T cell lines is summarized in Table 1.

TABLE 1
Available infinite T cells.

| Name | Characteristics |
|---|---|
| In1-L4a | mixed population of different subsets of T cells, Infinite CD3 T cells from donor 1 |
| | transduced with the pLV4a vector (PGK-Bcl6-2A-Bcl-XL expressing lentiviral vector). |
| In1-L4aJ3 | CD19 inCART, Infinite CD3 T cells from donor 1 transduced with the pLV4a vector and |
| | the pJ3 vector (An anti-CD19 CAR and hEGFRI expressing vector). |
| If1-L4a | Infinite CD4 (four) T cells from donor 1 with the pLV4a vector. |
| Ie1-L4a | Infinte CD8 (eight) T cells from donor 1 with the pLV4a vector. |
| Ie1-L4aJ3 | CD19 inCARTₑ, Infinite CD8 T cells from donor 1 transduced with the pLV4a vector and |
| | the pJ3 vector (An anti-CD19 CAR and hEGFRI expressing vector). |
| Igd1-L4a | Infinite gamma/delta T cells from donor 1 with the pLV4a vector. |
| Igd1-L4aJ3 | CD19 inCART_{gd}, Infinite gamma/delta T cells from donor 1 transduced with the pLV4a |
| | vector and the pJ3 vector (An anti-CD19 CAR and hEGFRI expressing vector). |

In1-L4a and the derived cells are readily maintained in regular culture medium, such as RPMI 1640 medium with GlutaMAX™ supplement, sodium pyruvate and 10% fetal bovine serum (FBS). In addition, 50-1000 IU/mL of recombinant human IL-2 is added for long-term growth (FIG. 1B). IL-15 also supported the proliferation, but IL7 or IL-21 did not support the proliferation (FIG. 1B). When suspension cultures were maintained with semi-weekly changes of medium, the cells could proliferate and expand very rapidly at an exponential pattern, with a doubling time of about 24 h. These infinite T cells were kept in culture and continued to proliferate for more than 3 months, with no change in the rate of proliferation in the presence of IL-2 (FIG. 1B).

The cells are highly dependent on IL-2 to survive and proliferate and stopped proliferating and died rapidly after withdrawal of IL-2 from the culture medium (FIG. 1B). The infinite T cells were CD3 positive, and other surface markers such as CD4 or CD8, TCRαβ or TCRgδ or CD16 were expressed on some subsets of infinite T cells, even after long-term culture and expansion in vitro (FIG. 1C). Those markers indicate that the infinite T cells were a mixed population of different subsets of T cells (FIG. 1C), therefore, a specific T cell population may be isolated by cell sorting using a specific T cell marker. For example, CD8⁺ infinite T cells were isolated by cell sorting using an anti-CD8 antibody. Another specific T cell population, the γδ T cell population was also isolated by cell sorting using an anti-TCRgδ antibody. After sorting, a relatively pure γδ T cell line was generated (FIG. 1D).

Mature T cells can further differentiate in the lymphoid tissues into distinct functional subsets such as Th1, Th2, Th17, Treg, and Tfh. The differentiation into these functional subsets is driven by unique master transcription factors. For example, Th1 differentiation is driven by Tbet, Th2 by GATA-3, Th17 by RORgt, Treg by Foxp3, and Tfh by BCL6. Thus, based on existing literature, expressing high levels of BCL6 in mature T cells would be expected to lead to a Tfh-like phenotype. However, this type of differentiation was not seen in infinite T cells, which was unexpected.

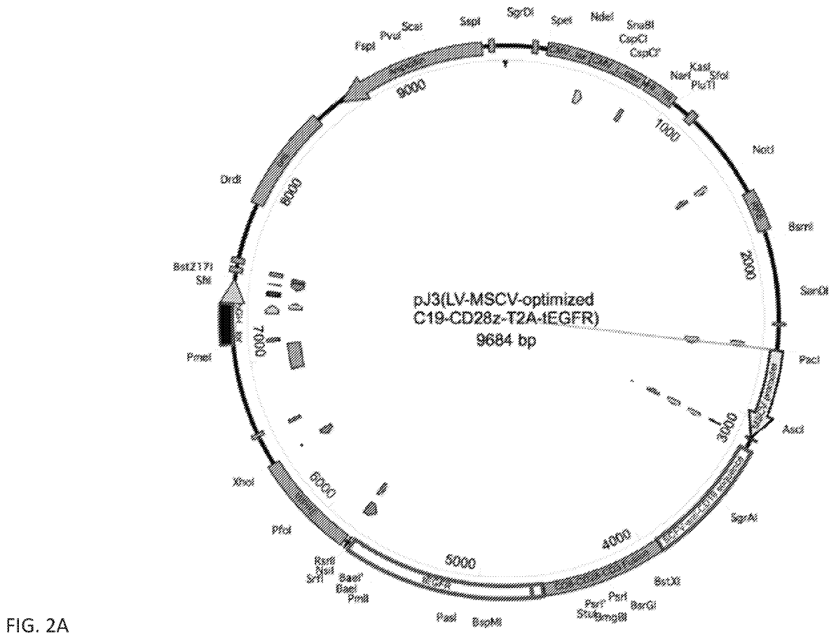

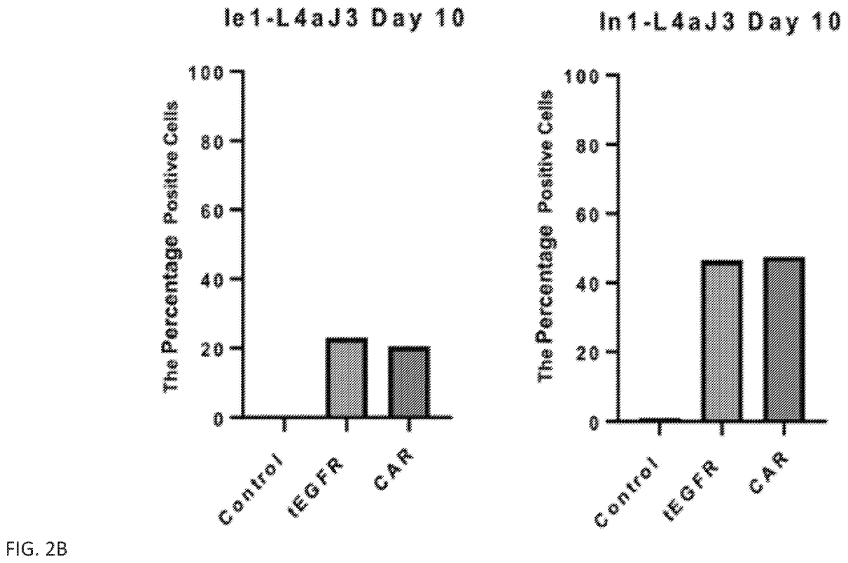

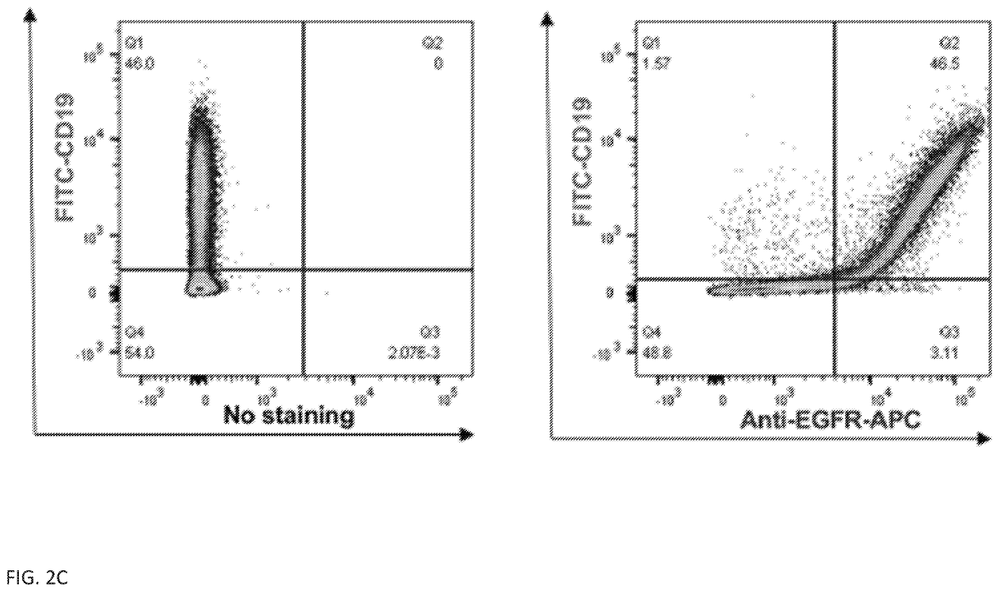

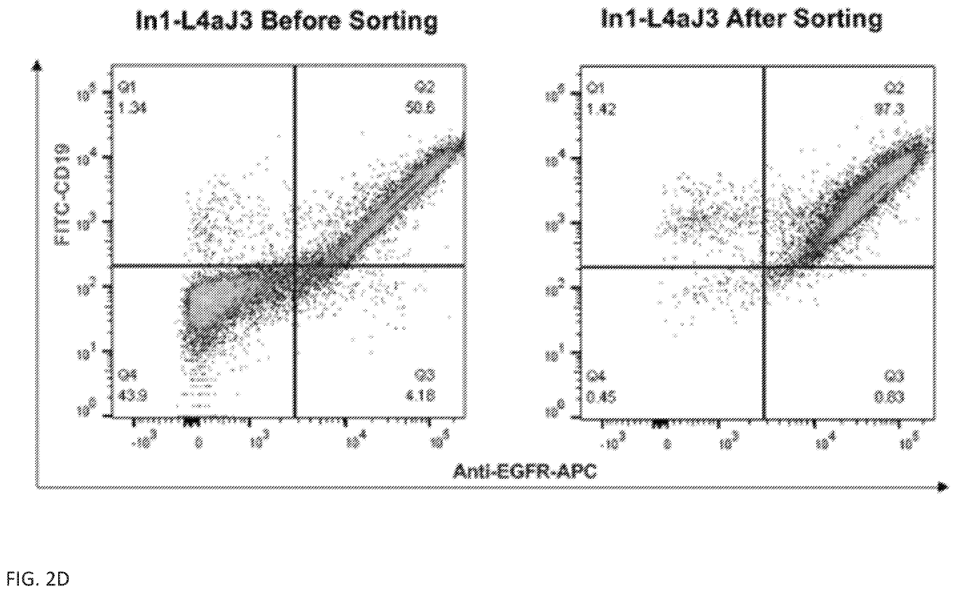

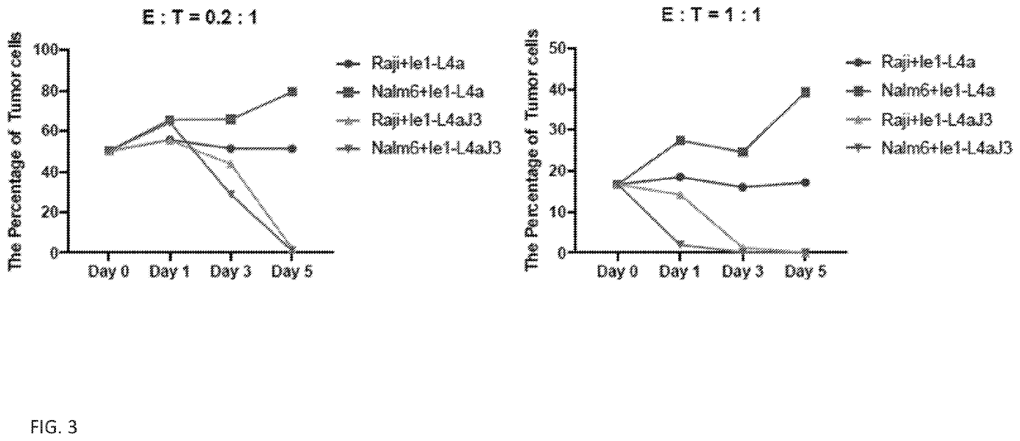

The cells were further modified to express anti-CD19 CAR to generate a series of ‘anti-CD19 infinite CAR T cells’ (CD19 inCART). The CD3 infinite T cells and CD8 infinite T cells, In1-L4a and Ie1-L4a, were modified to express on their surface a chimeric antigen receptor (CAR) targeting human CD19 using a vector designated as pJ3 plasmid (FIG. 2A), which resulted in In1-L4aJ3 and Ie1-L4aJ3 infinite T cell lines. Both In1-L4aJ3 and Ie1-L4aJ3 T cells expressed anti-CD19 CAR and could bind to recombinant human CD19 protein (FIGS. 2B and 2C). In1-L4aJ3 and Ie1-L4aJ3 infinite T cells were successfully generated and expanded in vitro, with similar proliferation rate as their parent cells. Ie1-L4aJ3 demonstrated the ability to lyse CD19 positive Raji lymphoma cell line and Nalm6 leukemia cell line in the presence of IL-2 at an effector:target ratio of 0.2:1 and 1:1 (FIG. 3).

### Example 2—Modification of the In1-L4a Derived T Cell Lines to Generate CD19 in CART Cells

The following example describes the modification of the In1-L4a derived infinite T cell lines to generate CD19 in CAR T cells. These procedures may similarly be used on other infinite T cells; however, for simplicity, the procedures are described in detail only with reference to In1-L4a and Ie1-L4a cell lines. One of skill in the art could adapt the method to insert the anti-CD19 CAR gene into other infinite cell lines, or to insert other CARs or TCRs targeting different tumor markers for therapeutic purposes against a variety of different tumors.

Recombinant lentiviral vector expressing anti-CD19 CAR and hEGFRt driven by MSCV promoter was generated by Gibson assembly method (NEB). The vector was designated as pJ3(LV-MSCV-optimized C19-CD28z-T2A-tEGFR) (FIG. 2A). The pJ3 plasmid and the lentiviral vector packaging mix (ABM) were co-transfected into 293T cells to produce the infectious pJ3 virus. One million of In1-L4a and Ie1-L4a cells described in Example 1 were transduced with pJ3 lentiviral vectors. 10 days after transduction, CAR positive cells were tested by flow cytometry using an AF647 labelled anti-EGFR antibody (R&D) and a FITC-labelled recombinant human CD19 protein (ACROBiosystems). The percentage of CAR positive cells in pJ3 transduced Ie1-L4a and In1-L4a group are about 20% and 46.5% (FIG. 2B).

The CAR positive percentages were further confirmed by double staining with FITC-labelled recombinant human CD19 protein and AF647 labelled cetuximab (FIG. 2C). The CAR positive cells were enriched by cell sorting using a cell sorter (BD). After sorting, relatively pure anti-CD19 CAR cells were collected and expanded in vitro (FIG. 2D). The In1-L4a and Ie1-L4a cells expressing CARs against human CD19 were designated as In1-L4aJ3 and Tel-L4aJ3. They exhibited a similar exponential proliferation rate as their parent In1-L4a and Ie1-L4a infinite T cells (FIG. 1B).

In vitro cytotoxicity of CD19 in CAR T cells against CD19 positive lymphoma and leukemia cells: Raji cell is a CD19+ B-cell lymphoma cell line derived from a Burkitt's lymphoma patient that is widely used in preclinical research in lymphoma, and Nalm6 is a CD19+ B-cell leukemia cell line derived from an acute lymphoblastic leukemia patient. Therefore, both of them were used to test the cytotoxic activity of the infinite anti-CD19 CART cell lines by co-culturing the effector and target cells in the presence of IL-2 at the ratio of 0.2:1 and 1:1. The test was performed in a 12-well plate. Briefly, 0.1 million of Raji or Nalm6 cells were cultured with 0.02 million or 0.1 million Ie1-L4aJ3 (anti-CD19 CART) or Ie1-L4a (No anti-CD19 CAR) cells per well in 2 mL of the above mentioned medium. After 5 days of co-culture, the cells in each well were stained with APC conjugated anti-CD8 antibody (BD) and cells were acquired using a BD Fotessa Analyser (BD) to determine the percentages of live T cells and tumor cells. The flow cytometry data was analyzed using the FlowJo software. The data demonstrated that both Ie1-L4aJ3 infinite T cells can efficiently lyse both Raji and Nalm6 tumor cells in vitro (FIG. 3). In contrast, no significant lysis of Raji or Nalm6 tumor cells was observed with Ie1-L4a cells as they lacked anti-CD19 CAR.

### Example 3—Infinite T Cells for Off-the-Shelf Adoptive T-Cell Therapies

Infinite T cells have the ability to proliferate rapidly and long-term. To date, we have generated infinite T cells by lentiviral transduction of BCL6 and BCL2L1 from 8 healthy donors and have observed that they can grow rapidly and continuously for >12 months in the presence of IL-2 or IL-15. Incorporation of an anti-CD19 CAR by lentivirus into these cells did not affect their growth rate. The fold increase in these T cells is ˜100-fold over 10 days and ˜1 million-fold over 30 days and their proliferative capacity is unchanged over 12 months of continuous in vitro culture (FIG. 5A). Phenotypically, the infinite T cells consisted of a mixture of CD4⁺ and CD8⁺ T cells, which could be sorted to high purity by magnetic beads (FIG. 5B). Foxp3⁺ cells were <5% within CD4⁺ T cells (data not shown). Withdrawal of cytokines at any point resulted in cell death rapidly within a week, suggesting that these T cells have not transformed into a malignant phenotype and do not develop the ability for autonomous growth (FIG. 5C).

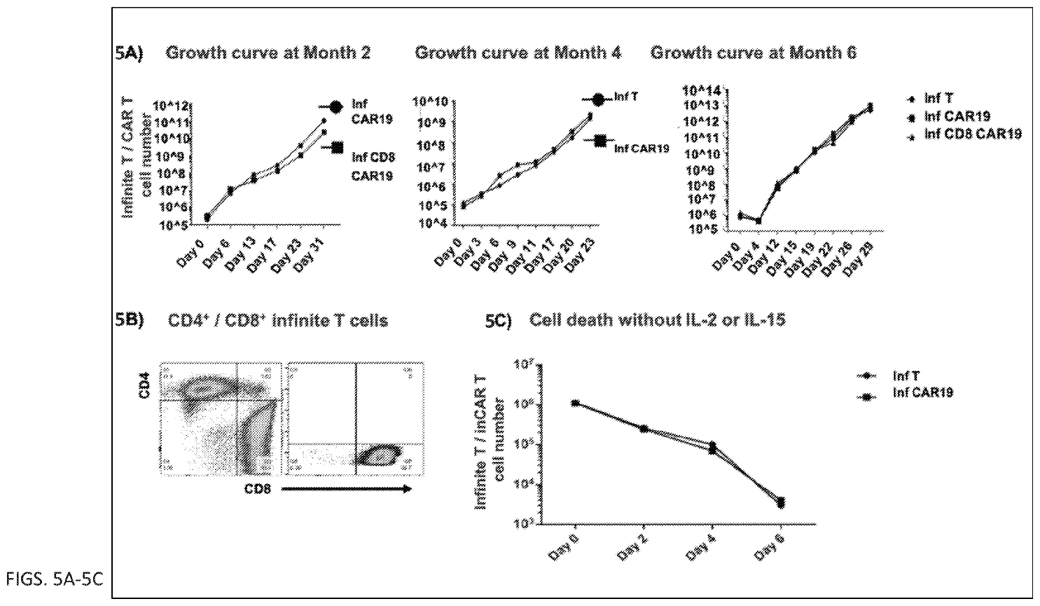

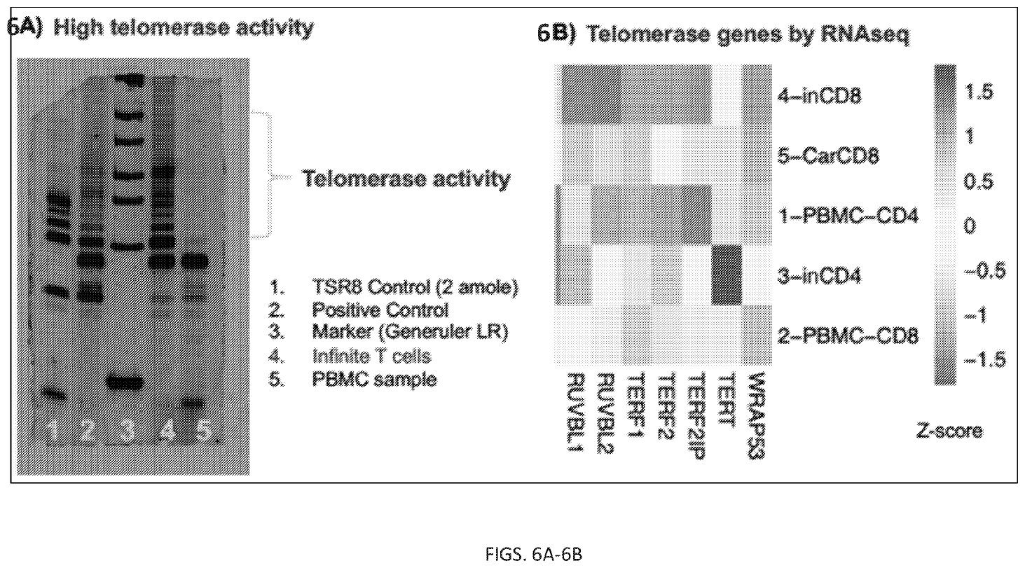

Infinite T cells exhibit high telomerase activity. Since proliferation of T cells after 30-40 population doublings leads to progressive shortening of telomeres and replicative senescence (Barsov et al., 2011), the inventors determined telomerase activity in these cells using the TRAPeze telomerase activity detection kit (Sigma). The hTERT activity in the infinite T cells was very high relative to the corresponding T cells from peripheral blood mononuclear cells (PBMC) (FIG. 6A). RNAseq analysis of these cells was consistent with this observation in infinite CD4⁺, infinite CD8⁺, and infinite CD8+CAR+ T cells (FIG. 6B). These results suggested that the transduced genes likely induce high telomerase activity in infinite T cells, which results in stabilization of telomere length, prevents replicative senescence, and confers the property of long-term proliferative capacity.

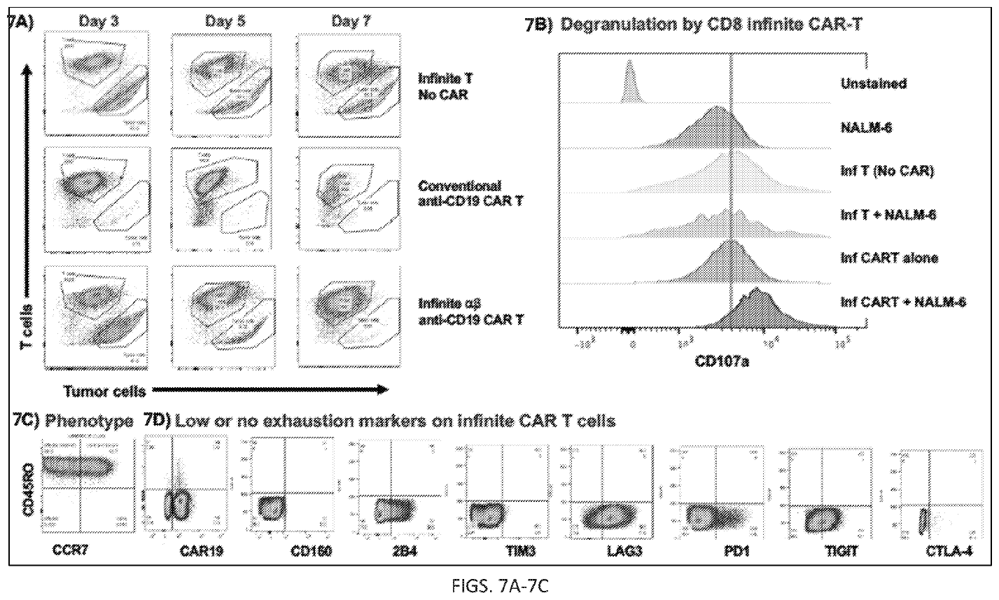

Incorporation of anti-CD19 CAR redirects the specificity of infinite T cells against B-cell malignancies. Lentiviral transduction of an anti-CD19 CAR (based on clone FMC63 anti-CD19 scFv with CD8a hinge/transmembrane domain, CD3t and CD28 signaling domains, and tEGFR as a transduction marker and safety switch (Wang et al., 2011)) into infinite T cells enabled them to efficiently and specifically degranulate and kill Daudi Burkitt lymphoma and NALM-6 acute B-cell lymphoblastic leukemia cell lines (FIGS. 7A-7B). Infinite T cells without CAR did not show any significant cytotoxicity or degranulation. As compared to conventional CAR T cells generated from freshly isolated T cells from healthy donors, infinite T cells were slower in killing tumor cells but almost completely eliminated them by day 7 (FIG. 7A). This slower killing may be a potential advantage in the clinic as it may cause less toxicity such as cytokine release syndrome and neurological toxicity. These anti-CD19 infinite CAR T cells had central and effector memory phenotype (FIG. 7C) and expressed very low or no markers associated with T-cell exhaustion (FIG. 7D).

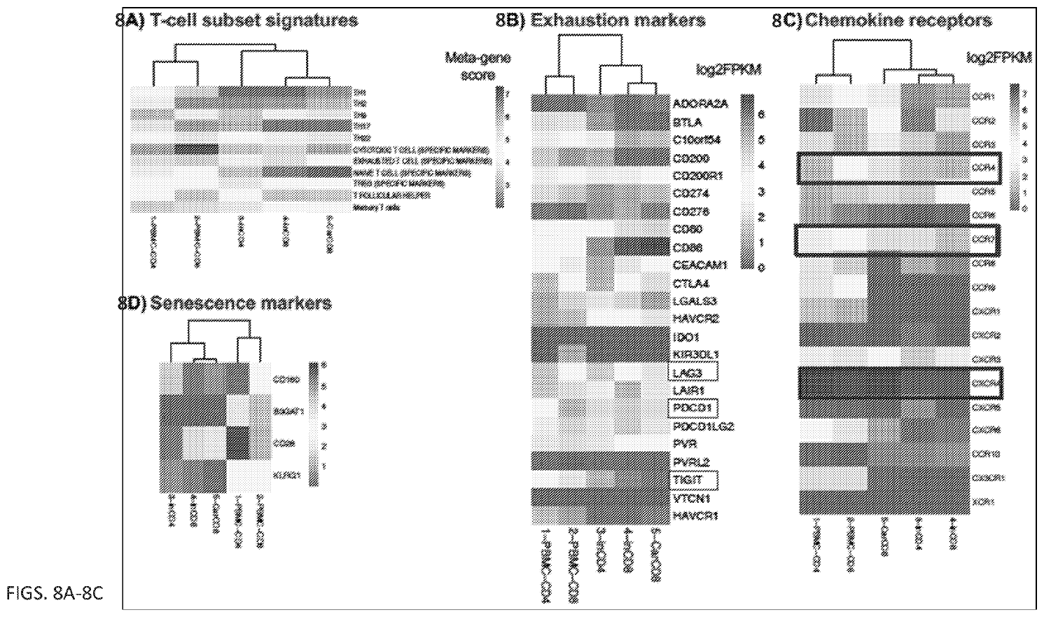

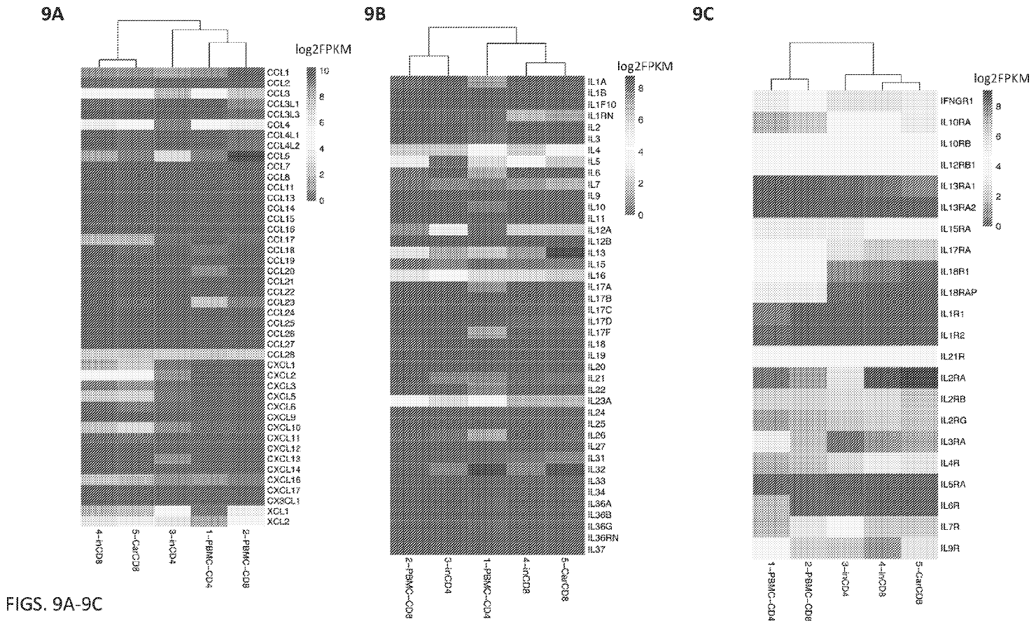

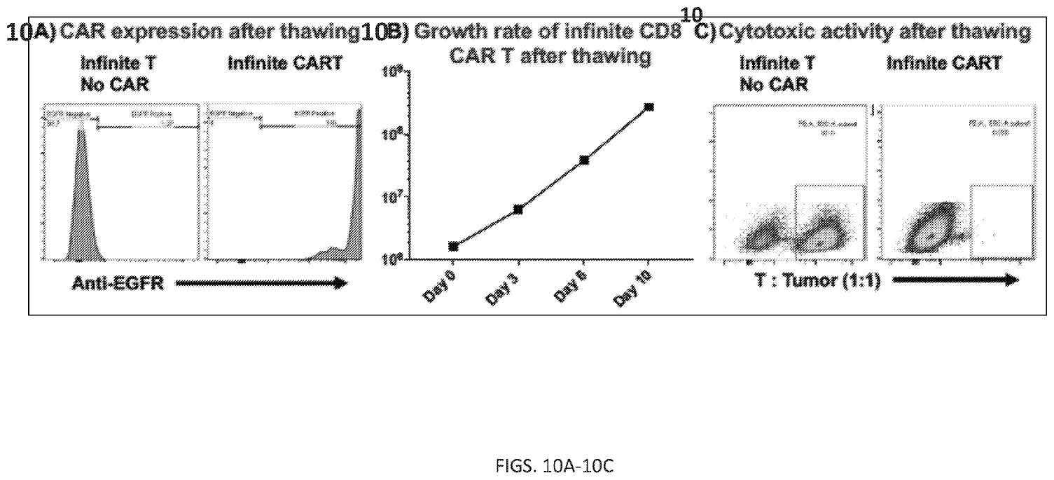

Transcriptional profile of infinite T cells. RNAseq analysis of infinite CD4⁺ and/or CD8⁺ T cells with or without anti-CD19 CAR compared with the corresponding CD4⁺ or CD8⁺ T cells isolated from PBMC samples was consistent with flow cytometry and functional data that these have memory and cytotoxic phenotype and do not express markers associated with classical T-cell exhaustion (FIGS. 8A-8B). Although they are generated by overexpressing BCL6, a master transcription factor for differentiation of naïve T cells to follicular helper T cells (T_{FH}),³ these cells do not exhibit a T_{FH} signature (FIG. 8A) and do not express high levels of CXCR5 (FIG. 8C) which is a hallmark of T_{FH} cells (Nurieva et al., 2009; Rawal et al., 2013). However, they retain the expression of chemokine receptors, CCR4 and CCR7 important for trafficking of T cells to lymph nodes, and CXCR4 important for trafficking to bone marrow (FIG. 8C) (Viola et al., 2006); both sites are commonly involved in lymphoma. The infinite T cells do not express senescence markers such as B3GAT1 (CD57), CD160, or KLRG1 (FIG. 8D) (Xu et al., 2017). The chemokine (FIG. 9A) and cytokine (FIG. 9B) gene expression profile was largely similar between infinite T cells and the corresponding CD4 or CD8 T cells derived from peripheral blood. Cytokine receptor gene expression showed some differences and included but not limited to increase in IL2RA, IL15RA, and IL21R levels and decrease in IL4R, IL7R, IL10RA, IL17RA, IL18R1, and IFNGR1 levels in infinite T cells compared to the corresponding CD4 or CD8 T cells derived from peripheral blood (FIG. 9C).

Infinite CAR T cells retain proliferative and cytotoxic function after freeze-thaw. Infinite T cells with and without CAR were cryopreserved and thawed after 6 months. After thawing they showed strong expression of CAR using anti-EGFR antibody (FIG. 10A). Culturing these cells in IL-2 showed ˜100-fold increase in cell number over 10 days and confirmed that the proliferative capacity of the infinite CD8 CAR T cells was maintained after freeze-thaw (FIG. 10B). In addition, these cells were shown to exhibit highly significant and specific cytotoxic activity against malignant B cells (FIG. 10C).

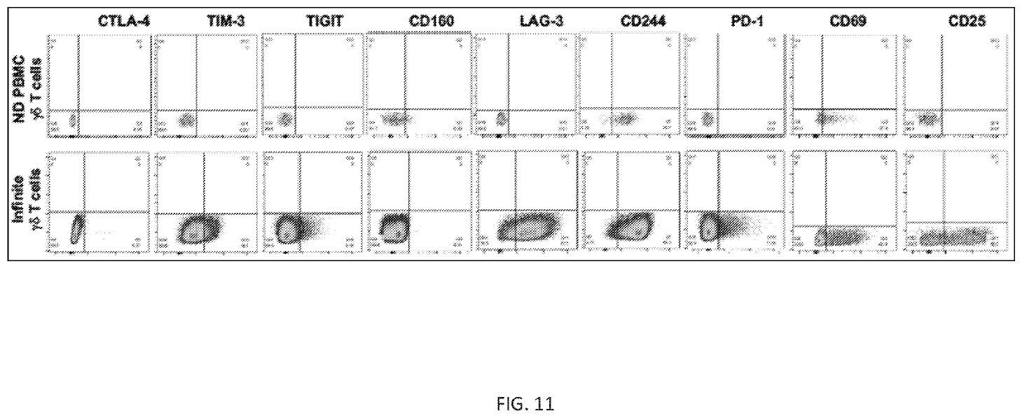

Infinite γδ T cells do not express exhaustion markers. Infinite γδ T cells did not significantly express markers of classical T-cell exhaustion (FIG. 11).

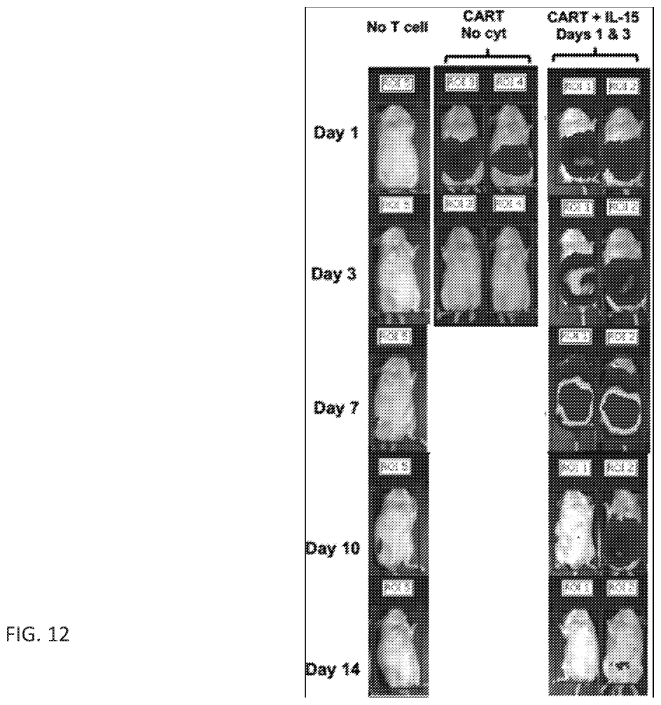

Anti-CD19 infinite CAR T cells exhibit antitumor efficacy in in vivo models. Using luciferase-labeled infinite CAR T cells, the inventors observed that following intraperitoneal (i.p.) injection into NSG mice, the T cells disappeared rapidly within 72 h without cytokine support (FIG. 12, middle column) when monitored by bioluminescence imaging (BLI), likely because mouse cytokines (both IL-2 and IL-15) do not support the growth of human T cells. In contrast, injection of recombinant human IL-15 on days 1 and 3 induced massive T cell proliferation with the cells persisting for 1 week after stopping IL-15 (FIG. 12, right column). These results suggested that IL-15 promotes in vivo proliferation and persistence but low doses might be sufficient. Similar effects were also observed with IL-2.

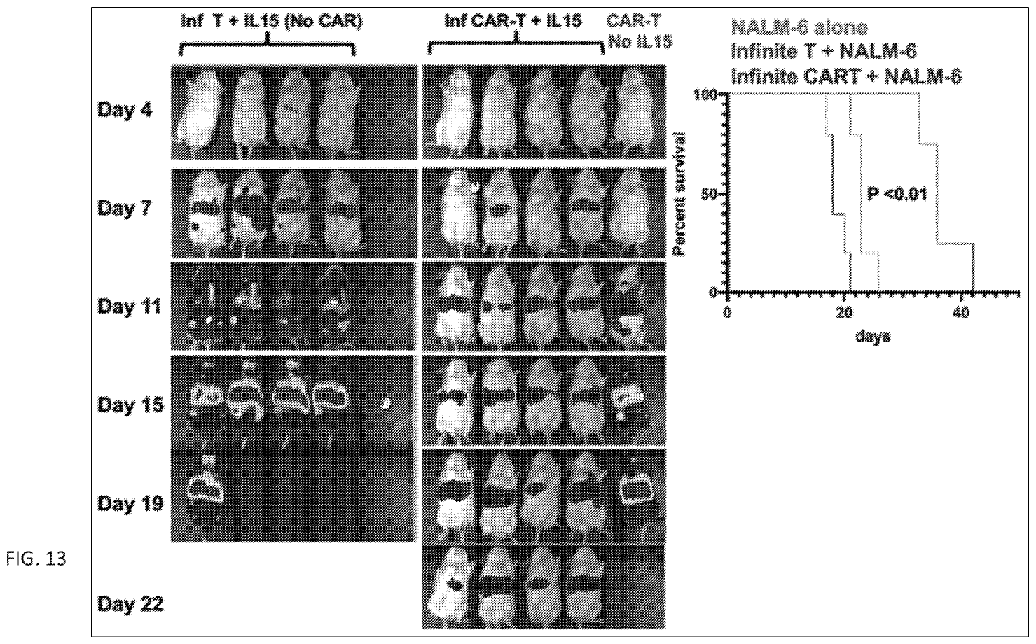

Next, the inventors injected luciferase-labeled NALM-6 tumor cells intravenously (IV) into NSG mice along with 3×10⁶ infinite T cells/mouse with or without CAR and injected IL-15 on days 0, 4, 7, and 11. There was significant tumor control as well as prolongation of survival in mice treated with infinite CAR T cells vs. infinite T cells without CAR (FIG. 13). Taken together, these results provided rationale to engineer the infinite T cells to secrete IL-2 or IL-15 to enhance their in vivo expansion and persistence.

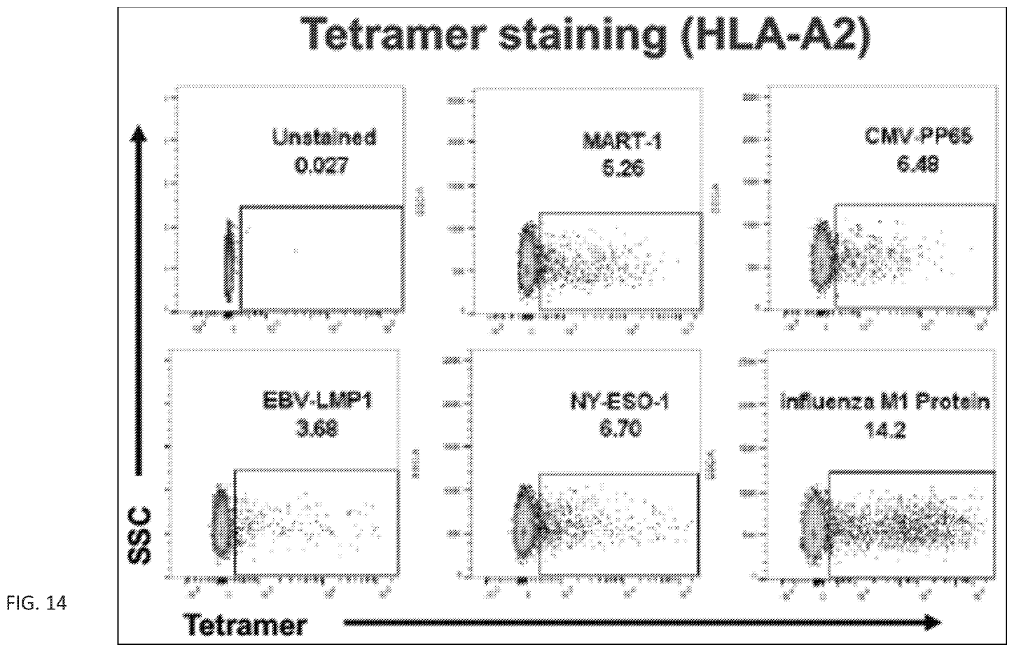

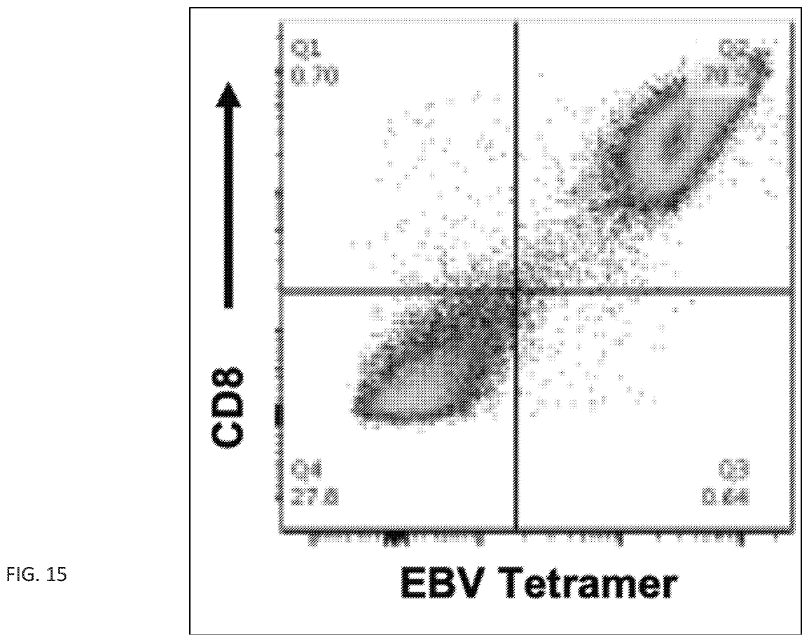

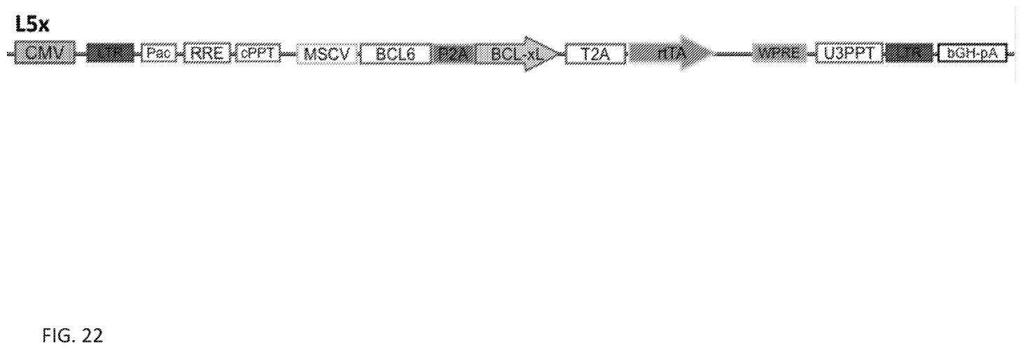

Microbial-assciated and tumor-associated antigen-specific infinite T cells. Testing of infinite T cells generated from an HLA-A2+ donor using tetramers revealed presence of a mixture of microbial- and tumor-associated antigen-specific T cells (FIG. 14). To generate an enriched population of these T cells, the inventors stimulated healthy donor peripheral blood mononuclear cells from an HLA-A2+ donor with a pool of peptides derived from EBV proteins. After 24 hours, CD137 positive T cells were sorted and used for generation of infinite T cells by transducing them with a BCL6 and BCL2L1 expressing lentiviral vector L5x (FIG. 22). The virus production and transduction protocol were described in example 1. Two weeks later after transduction, stimulate the transduced T cells with CD3/CD28/CD2 T cell activator again, then continue to culture them as described in example 1. After 7 weeks of culture and expansion in vitro in the presence of IL-2, 3 APC labeled tetramers including BMLF1-HLA-A2 tetramer were used to stain the expanded cells and enriched by APC enrichment magnetic beads, the enriched infinite T cells were cultured continuously like all other infinite T cells. At week 13, the enriched infinite T cells were stained with APC labeled BMLF1-HLA-A2 tetramer, about 70% of the T cells were found to be CD8 positive and BMLF1-HLA-A2 tetramer positive suggesting that they were specific against an HLA-A2-binding peptide (GLCTLVAML) derived from EBV-BMLF1 protein (FIG. 15). A similar approach can be used for generation of other antigen-specific T cells against microbial and tumor-associated antigens. Such antigen-specific T cells can in turn be used for transduction of CAR or TCR of interest to generate dual-antigen-specific T cells.

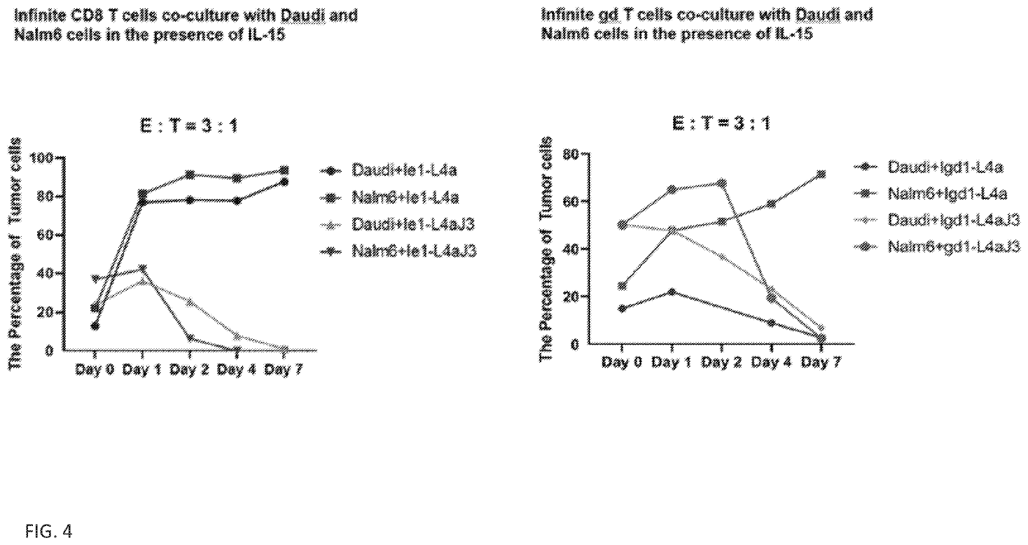

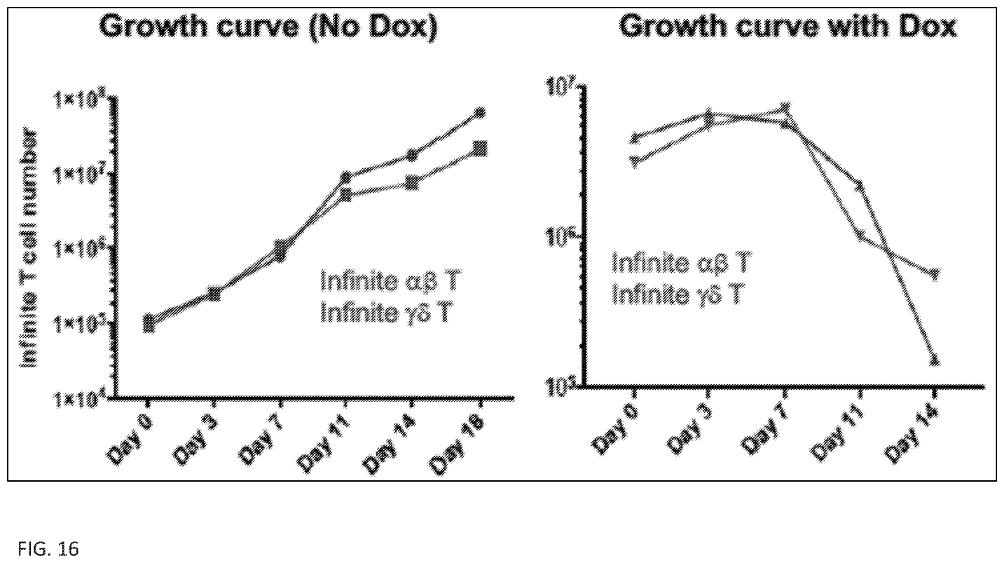

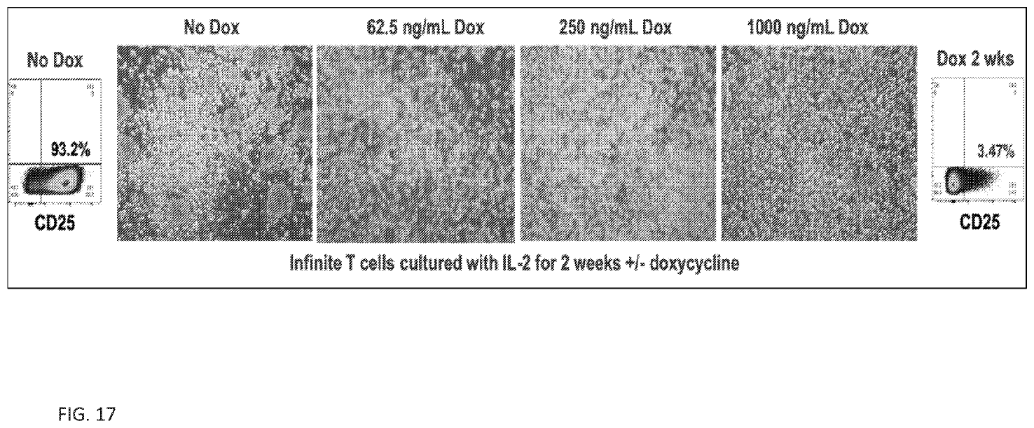

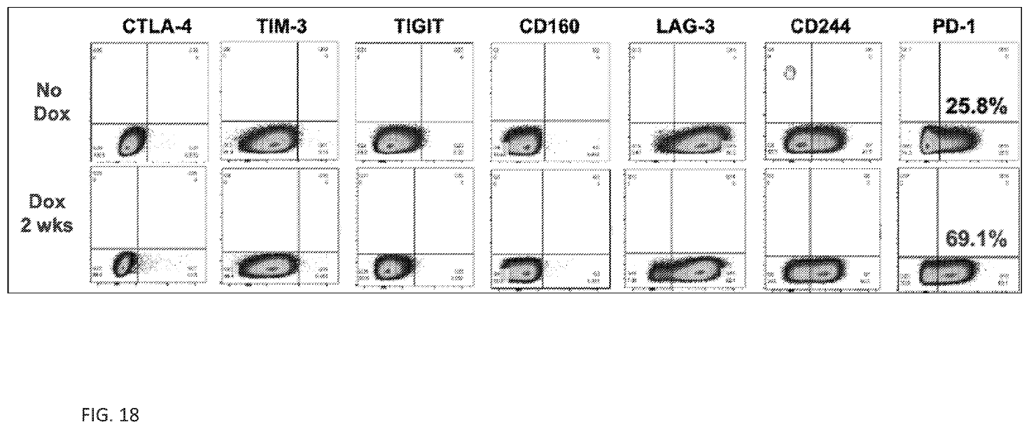

Tet-off system as a safety switch. The inventors have not observed any malignant transformation of the infinite T cells or cytokine-independent growth in vitro even in cultures from 6 to >12 months of infinite T cells derived from 8 donors (FIG. 4). However, to ensure safety for clinical translation, a Tet-off safety switch was incorporated that allows us to turn off the transduced BCL6 and BCL2L1 genes by using doxycycline. After incorporation of this Tet-off safety switch, infinite T cells maintained their growth rate in the absence of doxycycline but stopped proliferating and underwent gradual cell death in the presence of doxycycline at 1 μg/mL (FIG. 16), a concentration achievable with standard therapeutic dose of doxycycline in humans (Agwuh et al., 2006). By light microscopy imaging, the infinite T cells were found to gradual decrease in size along with decrease in proliferation clusters with increasing concentrations of doxycycline (FIG. 17). In addition, the CD25 expression decreased markedly in the presence of doxycycline (FIG. 17) and PD-1 expression increased suggesting that BCL6 and/or BCL2L1 genes likely controlled the expression of these molecules. Expression of other T-cell co-inhibitory receptors was not significantly altered in the presence of doxycycline (FIG. 18). A similar tet-off safety switch can also be used for control of IL-2 or IL-15 cytokine genes incorporated into infinite T cells.

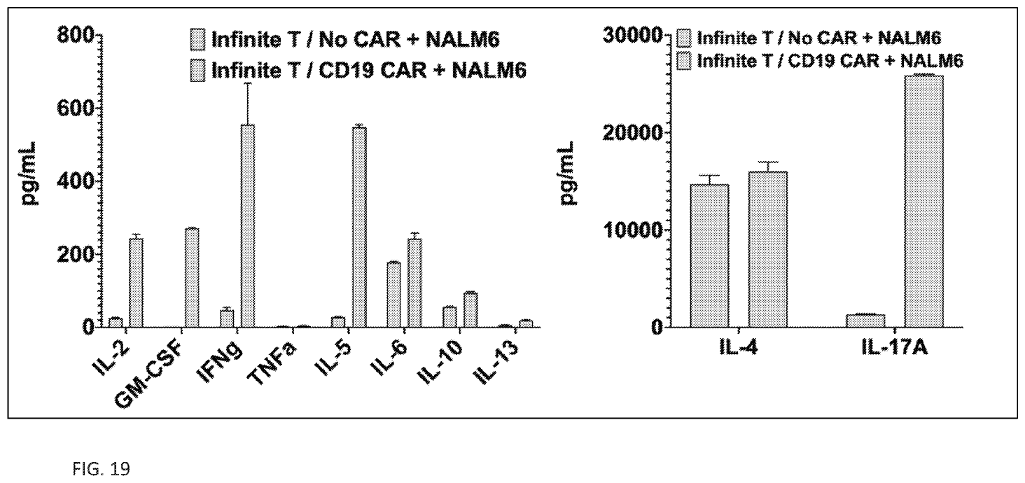

Anti-CD19 infinite CAR T cells produce effector cytokines in response to B-cell tumor cells. To determine the cytokine profile of infinite T cells produced in response to tumor cells, the inventors co-cultured NALM-6 tumor cells with CD8⁺ infinite T cells transduced with or without anti-CD19 CAR at an effector:target ratio of 5:1. After 3 days, cytokine levels were measured in the supernatants. The results show that infinite T cells with anti-CD19 CAR but not without predominantly produced significant amounts of IL-2, GM-CSF, IFN-γ, IL-5, and IL-17 in response to NALM-6 tumor cells (FIG. 19). Production of TNF-α, IL-4, IL-6, IL-10, or IL-13 by anti-CD19 infinite CAR T cells in response to tumor cells was minimal or not significantly different from infinite T cells without CAR expression. However, infinite T cells with or without CAR expression produced large amounts of IL-4 exceeding 10,000 pg/mL in the presence or absence of tumor cells (FIG. 19 and data not shown). This property of infinite T cells to constitutively produce large amounts of IL-4 in the absence of external stimulus may potentially have clinical application for treatment of various inflammatory disorders such as autoimmune diseases, graft-versus-host disease, certain types of infections associated with cytokine release syndrome, toxicities associated with CAR T-cell and other adoptive T-cell therapies, inflammatory bowel disorders, immune-related adverse events associated with various immunotherapies, hemophagocytic lymphohistiocytosis, periodic fever syndromes, etc., as IL-4 can suppress inflammation induced by T cells, macrophages, and other immune cells.

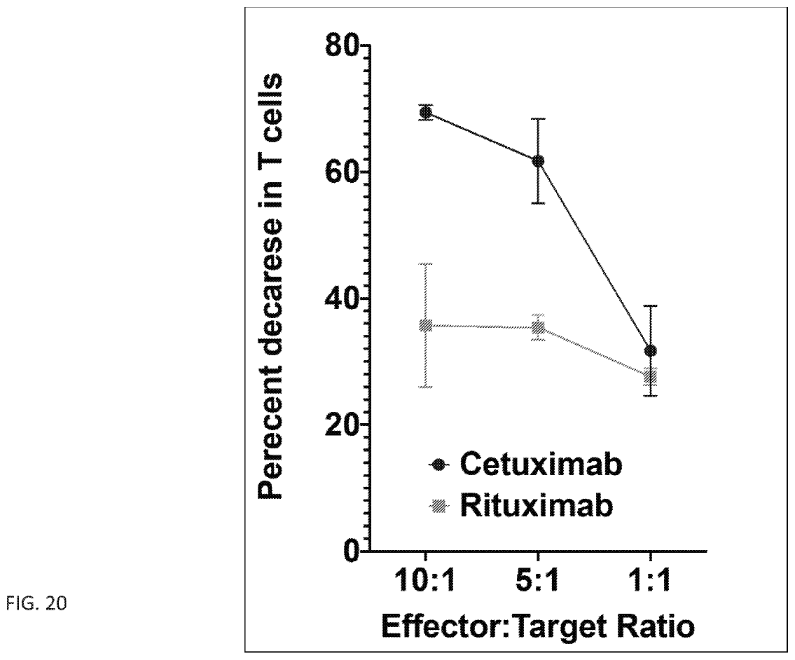

tEFGR safety switch for anti-CD19 infinite CAR T cells. To determine whether truncated EGFR (tEGFR) can serve as a safety switch for infinite T cells, the inventors cocultured infinite T cells expressing anti-CD19 CAR and tEGFR in the presence of cetuximab at a concentration of 5 μg/mL with or without natural killer (NK) cells isolated from healthy donor peripheral blood mononuclear cells. Cetuximab induced significant lysis of anti-CD19 infinite CAR T cells by antibody dependent cell-mediated cytotoxicity (ADCC) as compared to rituximab used as a control (FIG. 20). These results suggest that tEGFR may serve as a safety switch to eliminate infinite T cells in vivo in case of adverse events.

Generation of infinite T cells by transduction of BCL6 and BIRC5 genes. The inventors observed that infinite T cells may be generated by transduction of BCL6 and BCL2L1 genes or by transduction of BCL6 and BIRC5 genes into human T cells (FIG. 21A). While BCL2L1 encodes for Bcl-xL, an anti-apoptotic protein, BIRC5 encodes for survivin, an Inhibitor of Apoptosis (IAP) family protein that promotes proliferation and blocks apoptosis in cells. Transduction of either combination of genes resulted in generation of infinite T cells that have comparable long-term proliferative potential at an exponential growth rate in the presence of IL-2 (FIG. 21B). Moreover, these infinite T cells were generated with a Tet-off safety switch that allows us to turn off the transduced BCL6 and BCL2L1 or BCL6 and BIRC5 genes by using doxycycline. The vector also incorporated IL-15 gene that was transduced into these cells. Th cells grew at an exponential rate in the absence of doxycycline but stopped proliferating and underwent gradual cell death in the presence of doxycycline at 1 μg/mL despite IL-15 transduction and despite the addition of IL-2 to the culture medium (FIG. 21C).

One example of a construct L5x (MSCV-BCL6-P2A-BCL-xl-T2A-rtTA)) including BCL6 with Bcl-xl. The structure includes at least wild-type BCL-6 separated from BCL-xL by a P2A element, and BCL-xL is separated from rtTA (Tet on transactivator) by a T2A element (FIG. 22).

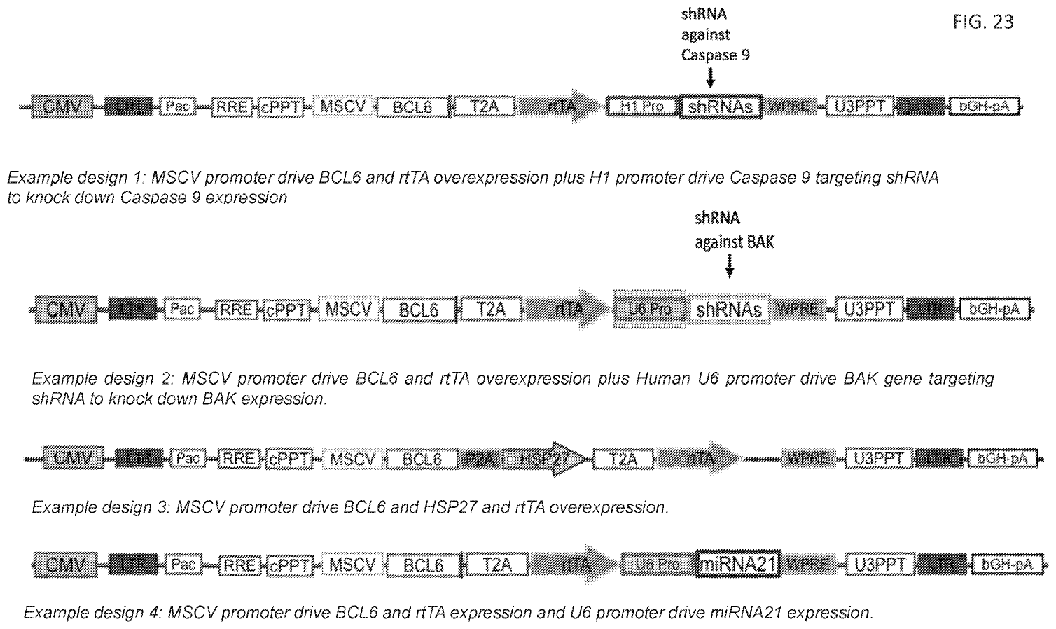

FIG. 23 provides multiple examples of embodiments of constructs that include at least BCL6; such examples may or may not utilize BCL-xL. As examples only, Example 1 utilizes a MSCV promoter to regulate BCL6 and rtTA overexpression, and the H1 promoter regulates Caspase 9-targeting shRNA to knock down Caspase 9 expression. Example 2 utilizes a MSCV promoter to regulate BCL6 and rtTA overexpression, in addition to the Human U6 promoter to regulate BAK gene-targeting shRNA to knock down BAK expression. In Example 3, the MSCV promoter regulates BCL6 and HSP27 and rtTA overexpression. In Example 4, the MSCV promoter regulates BCL6 and rtTA expression, and the U6 promoter regulates miRNA21 expression.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Agwuh K N, MacGowan A. Pharmacokinetics and pharmacodynamics of the tetracyclines including glycylcyclines. J Antimicrob Chemother. 2006; 58(2):256-265.
Ausubel et al., Current Protocols in Molecular Biology, Greene Publishing Associates and John Wiley & Sons, N Y, 1994.
Barsov E V. Telomerase and primary T cells: biology and immortalization for adoptive immunotherapy. Immunotherapy. 2011; 3(3):407-421.
Hooijberg, E., et al., J Immunol, 165(8): p. 4239-45, 2000.
Hurton, L. V., et al., Proc Natl Acad Sci USA, 113(48): p. E7788-e7797, 2016.
Migliaccio, M., et al., J Immunol, 165(9): p. 4978-84, 2000.
Nurieva R I, Chung Y, Martinez G J, et al. Bcl6 mediates the development of T follicular helper cells. Science. 2009; 325(5943):1001-1005.
Rawal S, Chu F, Zhang M, et al. Cross talk between follicular Th cells and tumor cells in human follicular lymphoma promotes immune evasion in the tumor microenvironment. J Immunol. 2013; 190(12):6681-6693.
Sambrook et al., Molecular Cloning: A Laboratory Manual, 3rd ed., Cold Spring Harbor Press, Cold Spring Harbor, N.Y. 2001.
Viola A, Contento R L, Molon B. T cells and their partners: The chemokine dating agency. Trends Immunol. 2006; 27(9):421-427.
Wang X, Chang W C, Wong C W, et al. A transgene-encoded cell surface polypeptide for selection, in vivo tracking, and ablation of engineered cells. Blood. 2011; 118(5):1255-1263.
Xu W, Larbi A. Markers of T Cell Senescence in Humans. Int J Mol Sci. 2017; 18(8).
WO2000/14257
WO2012/129514
WO2013/126726
WO2013/071154
WO2013/123061
WO2013/166321
WO2014/031687
WO2014/055668
US 2002131960
US 2013287748
US 20130149337
U.S. Pat. No. 6,410,319
U.S. Pat. No. 6,451,995
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,252,592
U.S. Pat. No. 8,324,353
U.S. Pat. No. 8,339,645
U.S. Pat. No. 8,398,282
U.S. Pat. No. 8,479,118
European patent application number EP2537416

What is claimed is:

1. A composition comprising immune cells engineered to express B-cell lymphoma 6 (BCL6) and one or more cell survival-promoting genes.

2. The composition of claim 1, wherein the cell survival-promoting gene is a pro-survival or anti-apoptotic gene.

3. The composition of claim 1, wherein immune cells are T cells, NK cells, innate lymphoid cells, or a mixture thereof.

4. The composition of any one of claims 1-3, wherein the cell survival-promoting gene is an anti-apoptotic B-cell lymphoma 2 (BCL-2) family gene.

5. The composition of claim 4, wherein the anti-apoptotic BCL-2 family gene is BCL2L1 (Bcl-xL), BCL-2, MCL1, BCL2L2 (Bcl-w), BCL2A1 (Bfl-1), BCL2L10 (BCL-B) or a combination thereof.

6. The composition of claim 5, wherein the anti-apoptotic BCL-2 family gene is BCL2L1 (Bcl-xL).

7. The composition of any one of claims 1-6, wherein the cell survival-promoting gene is an inhibitor of apoptosis family gene.

8. The composition of claim 7, wherein the inhibitor of apoptosis (IAP) family gene is XIAP, BIRC2 (C-IAPl), BIRC3 (C-IAP2), NAIP, BIRC5 (survivin), or a combination thereof.

9. The composition of any one of claims 1-8, wherein the cell survival-promoting gene is a nucleic acid polymer that inhibits or knocks out expression of one or more caspases.

10. The composition of claim 9, wherein the caspase is Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, or a combination thereof.

11. The composition of any one of claims 1-10, wherein the cell survival-promoting gene is a nucleic acid polymer that inhibits or knocks out expression of one or more pro-apoptotic genes.

12. The composition of claim 11, wherein the pro-apoptotic gene is BCL2L11 (BIM), BBC3 (PUMA), PMAIP1 (NOXA), BIK, BMF, BAD, HRK, BID, BAX, BAK1, BOK, or a combination thereof.

13. The composition of any one of claims 1-12, wherein the cell survival-promoting gene is a gene that has an anti-apoptotic effect.

14. The composition of claim 13, wherein the gene that has an anti-apoptotic effect is IGF1, HSPA4 (Hsp70), HSPB1 (Hsp27), CLAR (cFLIP), BNIP3, FADD, AKT, and NF-κB, RAF1, MAP2K1 (MEK1), RPS6KA1 (p90Rsk), JUN (C-Jun), BNIP2, BAG1, HSPA9, HSP90B1, miRNA21, miR-106b-25, miR-206, miR-221/222, miR-17-92, miR-133, miR-143, miR-145, miR-155, miR-330, or a combination thereof.

15. The composition of any one of claims 1-14, wherein the immune cells produce IL-4 in the absence of an external stimulus.

16. The composition of any one of claims 1-15, wherein the immune cells are engineered to express one or more cytokines.

17. The composition of claim 16, wherein the cytokine is IL-2 and/or IL-15.

18. The composition of any of claims 1-17, wherein the immune cells are derived from a donor that has not been diagnosed with cancer.

19. The composition of any of claims 1-18, wherein the immune cells are derived from an individual in need of treatment.

20. The composition of claim 18 or 19, wherein the donor is human.

21. The composition of any of claims 1-20, wherein the immune cells are T cells that are CD4+ T cells, CD8+ T cells, iNKT cells, NKT cells, γδ T cells, regulatory T cells, innate lymphoid cells, or a combination thereof.

22. The composition of any of claims 1-21, wherein the immune cells are T cells that comprise CD4-positive cells, CD8-positive cells, and/or γδ T cells.

23. The composition of any of claims 1-22, wherein the immune cells are T cells that are naïve T cells, effector T cells, memory T cells, stem cell memory T cells, terminally differentiated T cells, or a combination thereof.

24. The composition of any of claims 1-23, wherein the immune cells are T cells that are TCR αβ cells, TCR γδ T cells, or a combination thereof.

25. The composition of any of claims 1-24, wherein the immune cells are T cells that are Th1/Tc2, Th2/Tc2, Th9/Tc9, Th17/Tc17, Tfh, Th22, Tc22, or a combination thereof.

26. The composition of any of claims 1-25, wherein the immune cells express cytokines and cytotoxic molecules that are IFNγ, GM-CSF, TNFα, IL-2, IL-4, IL-5, IL-6, IL-9, IL-10, IL-13, IL-16, IL-17, IL-23, IL-32, granzyme B, perforin, or a combination thereof.

27. The composition of any of claims 1-26, wherein the immune cells are specific for one or more microbial antigens, one or more auto antigens, or one or more tumor antigens.

28. The composition of claim 27, wherein the virus is human immunodeficiency virus (HIV), herpes simplex virus (HSV), respiratory syncytial virus (RSV), cytomegalovirus (CMV), Epstein-Barr virus (EBV), Influenza A, Influenza B, Influenza C, vesicular stomatitis virus (VSV), Hepatitis B virus (HBV), Hepatitis C virus (HCV), Human papilloma virus (HPV), Varicella-zoster virus (VZV), vesicular stomatitis virus (VSV), polyomavirus, BK virus, JC virus, adenovirus, coronavirus, or a combination thereof.

29. The composition of any of claims 1-28, wherein the immune cells are engineered to express one or more chimeric antigen receptors (CAR) and/or one or more T cell receptors (TCR).

30. The composition of claim 29, wherein the CAR and/or TCR targets CD19, CD20, CD22, CD79a, CD79b, mesothelin, MAGE-A1, MAGE-A4, TCL1, NY-ESO, WT1, and/or BAFF-R antigen binding region.

31. The composition of claim 29 or 30, wherein the CAR comprises a partial or complete sequence from the hinge of CD8a, CD28, PD-1, CTLA4, alpha, beta or zeta chain of the T-cell receptor, CD2, CD3 zeta, CD3 epsilon, CD3 gamma, CD3 delta, CD45, CD4, CD5, CD8b, CD9, CD16, CD22, CD27, CD32, CD33, CD37, CD64, CD80, CD86, CD134, CD137, CD154, CD160, BTLA, LAIR1, TIGIT, TIM4, ICOS/CD278, GITR/CD357, NKG2D, LAG-3, PD-L1, PD-1, TIM-3, HVEM, LIGHT, DR3, CD30, CD224, CD244, SLAM, CD226, DAP, or a combination thereof or a synthetic molecule.

32. The composition of any one of claims 29-31, wherein the CAR comprises a partial or complete transmembrane domain from alpha chain of the T-cell receptor, beta chain of the T-cell receptor, zeta chain of the T-cell receptor, CD28, CD2, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, PD-1, CTLA4, DAP, a synthetic molecule, or a combination thereof.

33. The composition of any one of claims 29-32, wherein the CAR comprises one or more costimulatory domains from CD28, CD27, OX-40 (CD134), DAP10, DAP12, 4-1BB, or a combination thereof.

34. The composition of any of claims 1-33, wherein the composition comprises from 100,000 to 10 billion immune cells.

35. The composition of any of claims 1-34, wherein the immune cells comprise one or more safety switches.

36. The composition of claim 27, wherein the safety switch is truncated EGFR or fusion protein thereof.

37. The composition of any of claims 1-36, wherein the immune cells express IL-2, IL-15, one or more growth factors, one or more differentiation factors, or a combination thereof.

38. The composition of any of claims 1-37, wherein the cells maintain a proliferation rate for at least 3 months, 4 months, 5 months, 6 months, 7 months, 8 months, 9 months, 10 months, 11 months, or 12 months or more.

39. The composition of any of claims 1-38, wherein the immune cells have enhanced antitumor cytotoxicity, cytokine production, in vivo proliferation, in vivo persistence, and/or improved function.

40. A method for producing the immune cells of any of claims 1-39, comprising introducing one or more vectors encoding BCL6 and a cell survival-promoting gene to said cells.

41. The method of claim 40, wherein the cell survival-promoting gene is an anti-apoptotic B-cell lymphoma 2 (BCL-2) family gene.

42. The method of claim 41, wherein the anti-apoptotic BCL-2 family gene is BCL2L1 (Bcl-xL), BCL-2, MCL1, BCL2L2 (Bcl-w), BCL2A1 (Bfl-1), BCL2L10 (BCL-B), or a combination thereof.

43. The method of claim 42, wherein the anti-apoptotic BCL-2 family gene is BCL2L1 (Bcl-xL).

44. The method of any of claims 40-43, wherein the vector links BCL6 and Bcl-xL with a 2A sequence.

45. The method of any of claims 40-44, wherein the vector is a lentiviral vector.

46. The method of any one of claims 40-45, wherein introducing comprises transducing the cells with the lentiviral vector in the presence of IL-2, IL-15, and/or one or more other growth factors.

47. The method of claim 46, wherein IL-2 is at a concentration of 10 IU/mL to 1000 IU/mL.

48. The method of claim 46 or 47, wherein IL-2 is at a concentration of 400 IU/mL.

49. The method of any of claims 40-48, further comprising activating the T cells with CD3 and CD28.

50. The method of any of claims 40-49, further comprising culturing the cells in the presence of IL-2 and/or IL-15.

51. The method of claim 50, wherein the IL-2 or IL-15 are present at a concentration of 10-200 ng/mL.

52. The method of any of claims 40-51, wherein the cells are cultured for at least 3, 4, 5, 6, 7, 8, 9, 10, 11, 12, or more months with essentially no decrease in rate of proliferation.

53. The method of any of claims 40-52, further comprising sorting for a T cell subset.

54. The method of claim 53, wherein the T cell subset comprises CD4+ T cells, CD8+ T cells or γδ T cells.

55. The method of any one of claims 40-54, further comprising introducing one or more cytokines and/or one or more safety switches to the immune cells.

56. The method of claim 55, wherein the one or more cytokines and/or one or more safety switches are on the same vector as the BCL6 and cell survival-promoting gene.

57. The method of claim 55, wherein the one or more cytokines and/or one or more safety switches are on a different vector as the BCL6 and cell survival-promoting gene.

58. A composition comprising a population of cells of any one of claims 1-39 for the treatment of an immune-related disorder, infectious disease, and/or cancer, wherein the immune cells are targeted against one or more molecules.

59. A method of treating a disease or disorder in a subject comprising administering an effective amount of immune cells of any one of claims 1-39 to the subject.

60. The method of claim 59, wherein the disease or disorder is an infectious disease, cancer or immune-related disorder.

61. The method of claim 60, wherein the immune-related disorder is an autoimmune disorder, graft versus host disease, allograft rejection, or inflammatory condition.

62. The method of any one of claims 59-61, wherein the immune cells are allogeneic with respect to the subject.

63. The method of any one of claims 59-61, wherein the immune cells are autologous with respect to the subject.

64. The method of claim 60, wherein the disease is a cancer.

65. The method of claim 64, wherein the cancer is a solid cancer or a hematologic malignancy.

66. The method of claim 59, wherein the disease or disorder is an autoimmune disease, graft-versus-host disease, an infection associated with cytokine release syndrome, a toxicity associated with an immunotherapy, an inflammatory bowel disorder, an immune-related adverse event associated with an immunotherapy, hemophagocytic lymphohistiocytosis, periodic fever syndrome, or a combination thereof.

67. The method of claim 66, wherein the infection associated with cytokine release syndrome is from a coronavirus.

68. The method of claim 67, wherein the coronavirus is SARS-CoV, SARS-CoV-2, or MERS.

69. The method of any one of claims 59-68, wherein the immune cells produce IL-4 under conditions to suppress inflammation induced by T cells, macrophages, and/or other immune cells.

70. The method of any one of claims 59-69, further comprising administering at least a second therapeutic agent to the subject.

71. The method of claim 70, wherein the at least a second therapeutic agent comprises chemotherapy, immunotherapy, surgery, radiotherapy, drug therapy, targeted therapy, hormone therapy, biotherapy, or a combination thereof.


SEQUENCE LISTING (80 sequences)

SEQ ID NO: 1 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 2 - DNA, 2118 bp, Homo sapiens
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 3 - protein, 706 aa, Artificial Sequence
  Description of Artificial Sequence Synthetic polypeptide
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGLEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 4 - DNA, 2118 bp, Artificial Sequence
  Description of Artificial Sequence Synthetic polynucleotide
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcttgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 5 - protein, 21 aa, Artificial Sequence
  Description of Artificial Sequence Synthetic peptide
GSGEGRGSLLTCGDVEENPGP

SEQ ID NO: 6 - protein, 22 aa, Artificial Sequence
  Description of Artificial Sequence Synthetic peptide
GSGATNFSLLKQAGDVEENPGP

SEQ ID NO: 7 - protein, 23 aa, Artificial Sequence
  Description of Artificial Sequence Synthetic peptide
GSGQCTNYALLKLAGDVESNPGP

SEQ ID NO: 8 - protein, 25 aa, Artificial Sequence
  Description of Artificial Sequence Synthetic peptide
GSGVKQTLNFDLLKLAGDVESNPGP

SEQ ID NO: 9 - DNA, 2895 bp, Artificial Sequence
  Description of Artificial Sequence Synthetic polynucleotide
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgcggaagcggagctactaacttcagcctgctgaagcaggctggagacgtggaggagaaccctggacctagatctggaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 10 - DNA, 2892 bp, Artificial Sequence
  Description of Artificial Sequence Synthetic polynucleotide
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgcggaagcggagctactaacttcagcctgctgaagcaggctggagacgtggaggagaaccctggacctagatctggaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaa

SEQ ID NO: 11 - DNA, 10737 bp, Artificial Sequence
  Description of Artificial Sequence Synthetic polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatcctaggctagccaccatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgcggaagcggagctactaacttcagcctgctgaagcaggctggagacgtggaggagaaccctggacctagatctggaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaaacgcgtggcagtggcgagggtagaggttctctcctcacttgtggtgatgttgaagaaaaccctggtccaatgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaggtgaccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatgtctatcccgcccctaactccgcccagttccgcccattctccgccccatggctgactaattttttttatttatgcagaggccgaggccgcctcggcctctgagctattccagaagtagtgaggaggcttttttggaggccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 12 - DNA, 1071 bp, Artificial Sequence
  Description of Artificial Sequence Synthetic polynucleotide
atgctgctgctggtgaccagcctgctgctgtgcgagctgccacaccctgccttcctgaggaaagtgtgtaatggcatcggcatcggcgagtttaaggacagcctgtccatcaacgccacaaatatcaagcacttcaagaactgtacctctatcagcggcgacctgcacatcctgccagtggccttcagaggcgattcctttacacacaccccaccactggacccacaggagctggatatcctgaagacagtgaaggagatcaccggcttcctgctgatccaggcatggccagagaacaggacagatctgcacgcctttgagaatctggagatcatcagaggcaggaccaagcagcacggccagttctctctggccgtggtgagcctgaacatcacatccctgggcctgcgctctctgaaggagatcagcgacggcgatgtgatcatctccggcaacaagaatctgtgctatgccaacaccatcaattggaagaagctgtttggcacatctggccagaagaccaagatcatcagcaaccgcggcgagaattcctgcaaggcaaccggacaggtgtgccacgcactgtgtagccctgagggatgttggggaccagagccacgcgactgcgtgtcctgtaggaacgtgtctaggggaagggagtgcgtggataagtgtaatctgctggagggagagccaagggagttcgtggagaactccgagtgcatccagtgtcaccccgagtgcctgcctcaggccatgaacatcacatgtaccggccggggccctgacaattgcatccagtgtgcccactacatcgatggccctcactgcgtgaagacatgtccagccggcgtgatgggcgagaacaataccctggtgtggaagtatgcagacgcaggacacgtgtgccacctgtgtcaccccaattgcacatacggatgtaccggaccaggactggagggatgtcctacaaacggccctaagatcccaagcatcgcaaccggaatggtgggagcactgctgctgctgctggtggtggcactgggaatcggactgttcatgaggcggtga

SEQ ID NO: 13 - protein, 356 aa, Artificial Sequence
  Description of Artificial Sequence Synthetic polypeptide
MLLLVTSLLLCELPHPAFLRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRR

SEQ ID NO: 14 - DNA, 315 bp, Artificial Sequence
  Description of Artificial Sequence Synthetic polynucleotide
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 15 - DNA, 747 bp, Artificial Sequence
  Description of Artificial Sequence Synthetic polynucleotide
atgagccgcctggataagtccaaagtgatcaactctgccctggagctgctgaatgaagtgggcatcgagggcctgaccacacggaagctggcccagaagctgggagtggagcagccaaccctgtactggcacgtgaagaacaagcgcgccctgctggacgccctggccatcgagatgctggatcggcaccacacacacttctgccccctggagggagagtcctggcaggatttcctgcggaacaatgccaagagctttagatgtgcactgctgtcccacagggacggagcaaaggtgcacctgggcaccaggcctacagagaagcagtacgagaccctggagaaccagctggccttcctgtgccagcagggcttttctctggagaatgcactgtatgcactgagcgccgtgggacacttcaccctgggatgcgtgctggaggaccaggagcaccaggtggccaaggaggagagagagacacccaccacagattccatgccccctctgctgaggcaggccatcgagctgtttgaccaccagggagcagagcctgccttcctgtttggcctggagctgatcatctgcggcctggagaagcagctgaagtgtgagtctggaggaccagcagacgccctggacgatttcgacctggatatgctgcccgccgatgccctggacgattttgacctggatatgctgcctgccgacgccctggacgatctggacctggatatgctgccaggcacc

SEQ ID NO: 16 - protein, 248 aa, Artificial Sequence
  Description of Artificial Sequence Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDLDLDMLPG

SEQ ID NO: 17 - DNA, 747 bp, Artificial Sequence
  Description of Artificial Sequence Synthetic polynucleotide
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 18 - protein, 248 aa, Artificial Sequence
  Description of Artificial Sequence Synthetic polypeptide
MSRLDKSKVINGALELLNGVGIEGLTTRKLAQKLGVEQPTLYWHVKNKRALLDALPIEMLDRHHTHFCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPTDALDDFDLDMLPADALDDFDLDMLPADALDDFDLDMLPG

SEQ ID NO: 19 - DNA, 456 bp, Artificial Sequence
  Description of Artificial Sequence Synthetic polynucleotide
atgtatcggatgcaactcctcagctgcattgcgttgtcactcgcactcgtcacgaactctgcaccgacatctagtagtactaagaaaacacagttgcaactggagcacctgctgttggatttgcaaatgatccttaacgggatcaacaactacaaaaaccctaagctcacacgaatgcttactttcaagttttacatgccgaaaaaagccacagagctgaagcatcttcagtgccttgaagaggagcttaaacccctcgaggaggtactgaatctcgcgcaaagcaagaattttcatttgcggccccgggaccttatatcaaacattaacgtgatcgtgttggaactcaagggatcagagacgacatttatgtgcgagtacgctgacgagaccgctacaatcgtagagtttctcaataggtggatcacgttttgccaaagcatcatctcaacgctc

SEQ ID NO: 20 - DNA, 462 bp, Artificial Sequence
  Description of Artificial Sequence Synthetic polynucleotide
atgtataggatgcagctgctgtcctgcatcgccttgtccctggcccttgtgaccaacagcgccccaacctcctcctctaccaaaaaaacccaacttcagcttgagcatctcctcttggacctgcagatgatcctgaatggtataaacaactacaagaaccccaagctgacccggatgcttacattcaaattctatatgcctaaaaaggctacagagctgaagcacctgcagtgcctggaagaggagctgaagccactggaagaggtcctgaacttggcccagagcaagaactttcacctcaggcccagggacttgataagcaacataaatgtaatcgtcctggagctgaaggggtctgaaacaaccttcatgtgtgagtatgcagatgagaccgctaccatcgtggagttcctcaacagatggattacattttgtcaatccatcatcagcaccctgacatct

SEQ ID NO: 21 - protein, 152 aa, Artificial Sequence
  Description of Artificial Sequence Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTL

SEQ ID NO: 22 - DNA, 420 bp, Artificial Sequence
  Description of Artificial Sequence Synthetic polynucleotide
atgggcctgacctctcagctgctgccacccctgttctttctgctggcctgtgccggcaatttcgtgcacggcgccaactgggtgaatgtgatctctgacctgaagaagatcgaggatctgatccagagcatgcacatcgacgccaccctgtatacagagtccgatgtgcacccttcttgcaaggtgacagccatgaagtgttttctgctggagctgcaggtcatctctctggagagcggcgacgccagcatccacgataccgtggagaatctgatcatcctggccaacaatagcctgagctccaacggcaatgtgacagagtccggctgcaaggagtgtgaggagctggaggagaagaacatcaaggagttcctgcagtcctttgtgcacatcgtgcagatgtttatcaatacctcttga

SEQ ID NO: 23 - protein, 139 aa, Artificial Sequence
  Description of Artificial Sequence Synthetic polypeptide
MGLTSQLLPPLFFLLACAGNFVHGANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 225 aa, Artificial Sequence
  Description of Artificial Sequence Synthetic polypeptide
MAPRRARGCRTLGLPALLLLLLLRPPATRGITCPPPMSVEHADIWVKSYSLYSRERYICNSGFKRKAGTSSLTECVLNKATNVAHWTTPSLKCIRDGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 25 - DNA, 675 bp, Artificial Sequence
  Description of Artificial Sequence Synthetic polynucleotide
atggcacctagaagagccagaggatgtagaacactgggactgccagcgctccttcttttgttgctgctgagaccacctgcaactcgcggaatcacttgtcctcctcctatgagtgtggaacacgctgacatttgggtcaagtcctactctctgtattcccgggagagatatatatgtaactctggtttcaaacgcaaggcaggcaccagcagccttaccgagtgtgtgcttaacaaggcaacaaatgtggctcactggacaacaccttctctgaagtgcattagagatggaggcggaggatcaggtggaggaggttctggtgggggtggatcaaattgggtgaacgtaatttccgacctgaaaaagatcgaagatctcattcaaagcatgcatatcgatgccaccctctataccgagagcgatgtccacccatcctgcaaagttacggcgatgaaatgcttcctgctcgagctccaggttatttctctggagagcggggatgcctccatccacgatactgtcgagaacctcattattctggccaataactccctgtctagcaatggcaatgtgactgaatcaggttgcaaggagtgcgaggagctcgaagagaaaaacataaaagaattcctgcaatcctttgtccatatcgtacagatgtttatcaacaccagc

SEQ ID NO: 26 - DNA, 1482 bp, Artificial Sequence
  Description of Artificial Sequence Synthetic polynucleotide
atggccctgcctgtgacagccctgctgctgcctctggctctgctgctgcatgccgctagacccgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcaacaactactccagcaccacgaccaccaacacctgctccaactatcgcatctcaaccactttctctacgtccagaagcatgccgaccagctgcaggaggtgcagttcatacgagaggtctagatttcgcatgtgatatctacatctgggcaccattggctgggacttgtggtgtccttctcctatcactggttatcaccctttactgctgggttagaagtaaaagaagtaggctacttcatagtgattacatgaatatgactcctcgacgacctggtcccacccgtaagcattatcagccctatgcaccaccacgagatttcgcagcctatcgctccagagttaaatttagcagaagtgcagatgctcctgcgtataaacagggtcaaaaccaactatataatgaactaaatctaggacgaagagaagaatatgatgttttagataaaagacgtggtcgagatcctgaaatgggaggaaaacctagaagaaaaaatcctcaagaaggcctatataatgaactacaaaaagataagatggcagaagcttatagtgaaattggaatgaaaggagaacgtcgtagaggtaaaggtcatgatggtctttatcaaggtcttagtacagcaacaaaagatacatatgatgcacttcatatgcaagcacttccacctcgtttcgaagagcaaaaacttatc

SEQ ID NO: 27 - protein, 487 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 1461 bp, Artificial Sequence
  Description of Artificial Sequence Synthetic polynucleotide
atggccctgccagtgaccgccctgctgctgccactggcactgctgctgcacgcagcaaggccagacatccagatgacacagaccacaagctccctgtccgcctctctgggcgacagagtgaccatctcttgcagggccagccaggatatctccaagtatctgaattggtaccagcagaagcctgatggcacagtgaagctgctgatctatcacacctctagactgcacagcggcgtgccatccaggtttagcggctccggctctggcacagactactctctgaccatcagcaatctggagcaggaggatatcgccacctatttctgccagcagggcaacacactgccttacacctttggcggcggcacaaagctggagatcaccggcggcggcggctctggaggaggaggaagcggaggaggaggatccgaggtgaagctgcaggagagcggaccaggactggtggcacccagccagtccctgtctgtgacatgtaccgtgtccggcgtgtctctgccagactacggcgtgagctggatcagacagccacctaggaagggactggagtggctgggcgtgatctggggctccgagaccacatactataactccgccctgaagtctcggctgaccatcatcaaggacaacagcaagtcccaggtgtttctgaagatgaattccctgcagacagacgataccgccatctactattgcgccaagcactactattacggcggctcttatgccatggattactggggccagggcacaagcgtgaccgtgtctagcaccacaacccctgcaccaagaccaccaacaccagcacctaccatcgcaagccagcctctgtccctgaggccagaggcatgcaggccagcagcaggaggagcagtgcacaccaggggcctggacttcgcctgcgatatctacatctgggcaccactggcaggaacatgtggagtgctgctgctgtctctggtcatcaccctgtattgttgggtgagaagcaagagatccaggctgctgcacagcgactacatgaatatgacaccaaggagaccaggaccaaccaggaagcactatcagccttacgcacctccaagggacttcgcagcatataggagcagggtgaagttttctcgcagcgccgatgccccagcctatcagcagggccagaaccagctgtacaacgagctgaatctgggcaggcgcgaggagtacgacgtgctggataagaggagaggaagggatccagagatgggaggcaagcctaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctaga

SEQ ID NO: 29 - DNA, 63 bp, Artificial Sequence
  Description of Artificial Sequence Synthetic oligonucleotide
atggccctgccagtgaccgccctgctgctgccactggcactgctgctgcacgcagcaaggcca

SEQ ID NO: 30 - DNA, 321 bp, Artificial Sequence
  Description of Artificial Sequence Synthetic polynucleotide
gacatccagatgacacagaccacaagctccctgtccgcctctctgggcgacagagtgaccatctcttgcagggccagccaggatatctccaagtatctgaattggtaccagcagaagcctgatggcacagtgaagctgctgatctatcacacctctagactgcacagcggcgtgccatccaggtttagcggctccggctctggcacagactactctctgaccatcagcaatctggagcaggaggatatcgccacctatttctgccagcagggcaacacactgccttacacctttggcggcggcacaaagctggagatcacc

SEQ ID NO: 31 - DNA, 45 bp, Artificial Sequence
  Description of Artificial Sequence Synthetic oligonucleotide
ggcggcggcggctctggaggaggaggaagcggaggaggaggatcc

SEQ ID NO: 32 - DNA, 360 bp, Artificial Sequence
  Description of Artificial Sequence Synthetic polynucleotide
gaggtgaagctgcaggagagcggaccaggactggtggcacccagccagtccctgtctgtgacatgtaccgtgtccggcgtgtctctgccagactacggcgtgagctggatcagacagccacctaggaagggactggagtggctgggcgtgatctggggctccgagaccacatactataactccgccctgaagtctcggctgaccatcatcaaggacaacagcaagtcccaggtgtttctgaagatgaattccctgcagacagacgataccgccatctactattgcgccaagcactactattacggcggctcttatgccatggattactggggccagggcacaagcgtgaccgtgtctagc

SEQ ID NO: 33 - DNA, 135 bp, Artificial Sequence
  Description of Artificial Sequence Synthetic polynucleotide
accacaacccctgcaccaagaccaccaacaccagcacctaccatcgcaagccagcctctgtccctgaggccagaggcatgcaggccagcagcaggaggagcagtgcacaccaggggcctggacttcgcctgcgat

SEQ ID NO: 34 - DNA, 78 bp, Artificial Sequence
  Description of Artificial Sequence Synthetic oligonucleotide
atctacatctgggcaccactggcaggaacatgtggagtgctgctgctgtctctggtcatcaccctgtattgttgggtg

SEQ ID NO: 35 - DNA, 123 bp, Artificial Sequence
  Description of Artificial Sequence Synthetic polynucleotide
agaagcaagagatccaggctgctgcacagcgactacatgaatatgacaccaaggagaccaggaccaaccaggaagcactatcagccttacgcacctccaagggacttcgcagcatataggagc

SEQ ID NO: 36 - DNA, 336 bp, Artificial Sequence
  Description of Artificial Sequence Synthetic polynucleotide
agggtgaagttttctcgcagcgccgatgccccagcctatcagcagggccagaaccagctgtacaacgagctgaatctgggcaggcgcgaggagtacgacgtgctggataagaggagaggaagggatccagagatgggaggcaagcctaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctaga

SEQ ID NO: 37 - protein, 487 aa, Artificial Sequence; identical to SEQ ID NO: 27
  Description of Artificial Sequence Synthetic polypeptide

SEQ ID NO: 38 - protein, 21 aa, Artificial Sequence
  Description of Artificial Sequence Synthetic peptide
MALPVTALLLPLALLLHAARP

SEQ ID NO: 39 - protein, 107 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 40 - protein, 15 aa, Artificial Sequence
  Description of Artificial Sequence Synthetic peptide
GGGGSGGGGSGGGGS

SEQ ID NO: 41 - protein, 120 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 42 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 43 - protein, 26 aa, Artificial Sequence
  Description of Artificial Sequence Synthetic peptide
IYIWAPLAGTCGVLLLSLVITLYCWV

SEQ ID NO: 44 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 45 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1467 bp, Artificial Sequence
  Description of Artificial Sequence Synthetic polynucleotide
atgctgctgctcgtgacctccctgctgctgtgcgagctgccacaccctgccttcctgctgatccctgacatccagatgacccagaccacaagctccctgtccgcctctctgggcgacagagtgacaatctcttgtagggccagccaggatatctccaagtatctgaactggtaccagcagaagccagatggcaccgtgaagctgctgatctatcacacatctaggctgcacagcggagtgccatcccggtttagcggatccggatctggaaccgactactctctgacaatcagcaacctggagcaggaggatatcgccacctatttctgccagcagggcaataccctgccttacacatttggcggcggcacaaagctggagatcaccggcagcacatccggatctggcaagccaggatccggagagggatctaccaagggagaggtgaagctgcaggagagcggaccaggactggtggcacccagccagtccctgtctgtgacctgtacagtgtccggcgtgtctctgccagactacggcgtgagctggatcaggcagccacctaggaagggactggagtggctgggcgtgatctggggctccgagaccacatactataatagcgccctgaagtccagactgaccatcatcaaggataacagcaagtcccaggtgttcctgaagatgaattccctgcagaccgacgatacagccatctactattgcgccaagcactactattacggcggctcctatgccatggactactggggccagggcacctctgtgacagtgtctagcgccgccgccatcgaagtgatgtatccacccccttacctggataacgagaagagcaatggcaccatcatccacgtgaagggcaagcacctgtgcccatctcccctgttccctggcccaagcaagcccttttgggtgctggtggtggtgggaggcgtgctggcctgttattctctgctggtgacagtggccttcatcatcttttgggtgaggagcaagcggagcaggctgctgcacagcgactacatgaacatgaccccccggagacccggccctacaagaaagcactatcagccttacgcaccaccaagggacttcgcagcctatagaagcagggtgaagttttctcgcagcgccgatgcaccagcatatcagcagggacagaatcagctgtacaacgagctgaatctgggcaggcgcgaggagtacgacgtgctggataagaggagaggaagggatcctgagatgggaggcaagcctaggcgcaagaacccacaggagggcctgtataatgagctgcagaaggacaagatggccgaggcctactccgagatcggcatgaagggagagcggagaaggggcaagggacacgatggcctgtatcagggcctgtctaccgccacaaaggacacctacgatgccctgcacatgcaggccctgcctccacgg

SEQ ID NO: 47 - protein, 489 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1458 bp, Artificial Sequence
  Description of Artificial Sequence Synthetic polynucleotide
atgctgctgctcgtgacctccctgctgctgtgcgagctgccacaccctgccttcctgctgatccctgacatccagatgacccagaccacaagctccctgtccgcctctctgggcgacagagtgacaatctcttgtagggccagccaggatatctccaagtatctgaactggtaccagcagaagccagatggcaccgtgaagctgctgatctatcacacatctaggctgcacagcggagtgccatcccggtttagcggatccggatctggaaccgactactctctgacaatcagcaacctggagcaggaggatatcgccacctatttctgccagcagggcaataccctgccttacacatttggcggcggcacaaagctggagatcaccggcagcacatccggatctggcaagccaggatccggagagggatctaccaagggagaggtgaagctgcaggagagcggaccaggactggtggcacccagccagtccctgtctgtgacctgtacagtgtccggcgtgtctctgccagactacggcgtgagctggatcaggcagccacctaggaagggactggagtggctgggcgtgatctggggctccgagaccacatactataatagcgccctgaagtccagactgaccatcatcaaggataacagcaagtcccaggtgttcctgaagatgaattccctgcagaccgacgatacagccatctactattgcgccaagcactactattacggcggctcctatgccatggactactggggccagggcacctctgtgacagtgtctagcatcgaagtgatgtatccacccccttacctggataacgagaagagcaatggcaccatcatccacgtgaagggcaagcacctgtgcccatctcccctgttccctggcccaagcaagcccttttgggtgctggtggtggtgggaggcgtgctggcctgttattctctgctggtgacagtggccttcatcatcttttgggtgaggagcaagcggagcaggctgctgcacagcgactacatgaacatgaccccccggagacccggccctacaagaaagcactatcagccttacgcaccaccaagggacttcgcagcctatagaagcagggtgaagttttctcgcagcgccgatgcaccagcatatcagcagggacagaatcagctgtacaacgagctgaatctgggcaggcgcgaggagtacgacgtgctggataagaggagaggaagggatcctgagatgggaggcaagcctaggcgcaagaacccacaggagggcctgtataatgagctgcagaaggacaagatggccgaggcctactccgagatcggcatgaagggagagcggagaaggggcaagggacacgatggcctgtatcagggcctgtctaccgccacaaaggacacctacgatgccctgcacatgcaggccctgcctccacgg

SEQ ID NO: 49 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 50 - DNA, 117 bp, Artificial Sequence
  Description of Artificial Sequence Synthetic polynucleotide
atcgaagtgatgtatccacccccttacctggataacgagaagagcaatggcaccatcatccacgtgaagggcaagcacctgtgcccatctcccctgttccctggcccaagcaagccc

SEQ ID NO: 51 - protein, 27 aa, Artificial Sequence
  Description of Artificial Sequence Synthetic peptide
FWVLVVVGGVLACYSLLVTVAFIIFWV

SEQ ID NO: 52 - protein, 463 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSQVPTAHPSPSPRPAGQFQTLVVGVVGGLLGSLVLLVWVLAVIERSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 1389 bp, Artificial Sequence
  Description of Artificial Sequence Synthetic polynucleotide
atgctactgctggtgaccagcctcctgctgtgcgagctgccccaccccgcgttcctgctcatccccgacatccagatgacccagacgacctcctcgctgagtgcatcactgggagaccgcgtcaccatctcatgccgagcttcccaggacatttccaagtacctgaactggtaccagcagaagcctgacggcaccgtcaagctgcttatctaccacactagtcgcctccactctggcgtgccctctagatttagtggctccggctcgggcaccgactacagcctgaccatcagcaacctggaacaggaggacatagccacttacttctgccagcagggcaacaccctgccctataccttcggcggcggcaccaagctggagatcacgggttcgacctccggatctgggaagccggggtccggagagggctccactaagggtgaggtgaagctccaggagagcgggcctgggctggtagcgcccagccagagcttatccgtgacctgtaccgtgtcgggagtctcgctgcctgattacggcgtgagctggattcgccagccgccccgcaaaggcttggaatggctaggtgtgatctggggctccgagaccacctattacaactccgccctgaagtcccggcttacgatcatcaaggacaactccaagtctcaggtgttcttgaagatgaactctcttcaaacagatgacaccgccatctattactgtgccaagcactactactacggcggcagctacgccatggattattggggccaaggaacttctgttacagtttcctctcaggtcccaacagcgcatccctctccaagcccgcgtcccgctggacagttccagactctggtggtgggcgtggtgggcgggctgctgggttctttggtgctgctggtgtgggtcctcgctgtcattgagcgcagcaagcgcagccgcctgttgcacagcgattacatgaatatgactccgcgccggcctggcccaacgcgtaagcactaccagccgtacgcgcccccgagagacttcgctgcatacaggtcccgcgtaaaattttcgcgctctgcggacgctcctgcctatcagcagggtcagaaccagctgtacaatgagctcaacctgggccgtagggaggagtacgatgtgctcgacaaacgccgtggtcgggacccggagatgggcggtaaacctcggcgcaagaatcctcaggagggcctttacaacgagctgcagaaggacaaaatggccgaggcctactccgagatcggtatgaagggggaacgccgtcgcggcaagggccacgatggattgtatcagggcctgtccaccgccaccaaggacacctacgacgccctgcatatgcaggccttgccgccccgc

SEQ ID NO: 54 - protein, 21 aa, Artificial Sequence
  Description of Artificial Sequence Synthetic peptide
QVPTAHPSPSPRPAGQFQTLV

SEQ ID NO: 55 - protein, 21 aa, Artificial Sequence
  Description of Artificial Sequence Synthetic peptide
VGVVGGLLGSLVLLVWVLAVI

SEQ ID NO: 56 - protein, 453 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVIDPEPCPDSDFLLWILAAVSSGLFFYSFLLTARSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 1359 bp, Artificial Sequence
  Description of Artificial Sequence Synthetic polynucleotide
atgttactgctcgttacttcgctgctgctgtgcgagctgccacaccccgcgttcttgctgattccggatatccagatgacccagacgacctcctccctctccgctagtctgggggaccgcgtgaccatctcatgccgagcttcccaggacatctctaagtacctgaactggtaccaacagaagcccgatgggaccgtgaagttgctcatttaccacacctctcgtctacacagtggtgtcccttctcgcttctcgggatccggttctggtacagattactccttgaccatctcaaatcttgaacaggaggacatcgccacttatttctgtcagcagggcaacacgcttccgtacaccttcggcggcggtactaagctggagatcaccggctcgaccagcggctcgggcaagcccggctccggcgaaggcagcaccaagggcgaggtgaagctccaggagagcggacccggactggtggcgccaagccagagcctgtctgtgacctgcaccgtgtccggcgtatctctgcccgactacggcgttagttggatccgccagccgccccgcaaaggcctggagtggctaggggtcatatggggctccgagaccacatactacaacagcgcactgaaatcccgcttgaccatcatcaaggacaacagcaagagccaggtgttcctgaagatgaattccttgcagactgatgacaccgccatctattactgtgctaagcactattactacggtggcagctacgcgatggattattggggccagggaacttctgtgacggtgtcctccgtgattgacccggagccatgtcctgacagtgacttcctgctttggatcctggccgctgtctcttctggccttttcttttactccttcctgctgacagccaggagcaagcgcagccgcctgttgcactccgactacatgaacatgactcctcgccgccccgggccaacccgcaagcactaccaaccctatgctcccccgcgcgactttgcggcctacagatcacgagtcaaatttagccgctcggcggacgctcctgcctaccagcagggacagaaccagctttacaacgagctcaacctgggcagaagggaggagtacgatgtgctggacaagcgtcgcggccgggaccccgagatgggcggtaagcctcggcgcaagaaccctcaggagggcctgtacaacgagctgcagaaggacaaaatggccgaggcttattcggaaatcggtatgaagggggagcggcgtcgtggcaaaggtcatgacggcctctaccaggggctgtccaccgccaccaaagatacctacgacgcattacatatgcaggccctgccgccgagg

SEQ ID NO: 58 - protein, 22 aa, Artificial Sequence
  Description of Artificial Sequence Synthetic peptide
MLLLVTSLLLCELPHPAFLLIP

SEQ ID NO: 59 - protein, 11 aa, Artificial Sequence
  Description of Artificial Sequence Synthetic peptide
VIDPEPCPDSD

SEQ ID NO: 60 - protein, 21 aa, Artificial Sequence
  Description of Artificial Sequence Synthetic peptide
FLLWILAAVSSGLFFYSFLLT

SEQ ID NO: 61 - DNA, 577 bp, Artificial Sequence
  Description of Artificial Sequence Synthetic polynucleotide
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 62 - DNA, 181 bp, Artificial Sequence
  Description of Artificial Sequence Synthetic polynucleotide
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 63 - DNA, 45 bp, Artificial Sequence
  Description of Artificial Sequence Synthetic oligonucleotide
tgagtacgccaaaaattttgactagcggaggctagaaggagagag

SEQ ID NO: 64 - DNA, 234 bp, Artificial Sequence
  Description of Artificial Sequence Synthetic polynucleotide
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 65 - DNA, 16 bp, Artificial Sequence
  Description of Artificial Sequence Synthetic oligonucleotide
aaaagaaaagggggga

SEQ ID NO: 66 - DNA, 516 bp, Artificial Sequence
  Description of Artificial Sequence Synthetic polynucleotide
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcat

SEQ ID NO: 67 - DNA, 2118 bp, Artificial Sequence; identical to SEQ ID NO: 2
  Description of Artificial Sequence Synthetic polynucleotide

SEQ ID NO: 68 - DNA, 66 bp, Artificial Sequence
  Description of Artificial Sequence Synthetic oligonucleotide
ggaagcggagctactaacttcagcctgctgaagcaggctggagacgtggaggagaaccctggacct

SEQ ID NO: 69 - DNA, 708 bp, Artificial Sequence
  Description of Artificial Sequence Synthetic polynucleotide
agatctggaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaa

SEQ ID NO: 70 - DNA, 63 bp, Artificial Sequence
  Description of Artificial Sequence Synthetic oligonucleotide
ggcagtggcgagggtagaggttctctcctcacttgtggtgatgttgaagaaaaccctggtcca

SEQ ID NO: 71 - DNA, 752 bp, Artificial Sequence
  Description of Artificial Sequence Synthetic polynucleotide
atgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaggtga

SEQ ID NO: 72 - DNA, 588 bp, Artificial Sequence
  Description of Artificial Sequence Synthetic polynucleotide
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 73 - DNA, 16 bp, Artificial Sequence
  Description of Artificial Sequence Synthetic oligonucleotide
aaaagaaaagggggga

SEQ ID NO: 74 - DNA, 181 bp, Artificial Sequence; identical to SEQ ID NO: 62
  Description of Artificial Sequence Synthetic polynucleotide

SEQ ID NO: 75 - DNA, 204 bp, Artificial Sequence
  Description of Artificial Sequence Synthetic polynucleotide
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 76 - DNA, 136 bp, Artificial Sequence
  Description of Artificial Sequence Synthetic polynucleotide
atcccgcccctaactccgcccagttccgcccattctccgccccatggctgactaattttttttatttatgcagaggccgaggccgcctcggcctctgagctattccagaagtagtgaggaggcttttttggaggcc

SEQ ID NO: 77 - DNA, 589 bp, Artificial Sequence
  Description of Artificial Sequence Synthetic polynucleotide
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 78 - DNA, 861 bp, Artificial Sequence
  Description of Artificial Sequence Synthetic polynucleotide
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 79 - DNA, 29 bp, Artificial Sequence
  Description of Artificial Sequence Synthetic oligonucleotide
attgtctcatgagcggatacatatttgaa

SEQ ID NO: 80 - protein, 9 aa, Epstein-Barr virus
GLCTLVAML